SEQUENCE LISTING (154 sequences)

SEQ ID NO: 1 - protein, 1367 aa, Artificial sequence
  Synthetic
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 37 aa, Artificial sequence
  Synthetic
AAVKEFFGSSQLSQFMDQNNPLSEITHKRRISALGPG

SEQ ID NO: 3 - DNA, 111 bp, Artificial sequence
  Synthetic
gcagcagtgaaagagttcttcggttccagccagctgtctcagtttatggaccagaacaacccgctgtctgagattacgcacaaacgtcgtatctccgcactcggcccaggc

SEQ ID NO: 4 - DNA, 23 bp, Artificial sequence
  Synthetic
cgtatctccgcactcggcccagg

SEQ ID NO: 5 - DNA, 23 bp, Artificial sequence
  Synthetic
cgtatcttcgcactcggcccagg

SEQ ID NO: 6 - DNA, 51 bp, Artificial sequence
  Synthetic
ccctggcccaccctcgtgaccaccctgacctgcggcgtgcagtgcttcagc

SEQ ID NO: 7 - protein, 17 aa, Artificial sequence
  Synthetic
PWPTLVTTLTCGVQCFS

SEQ ID NO: 8 - DNA, 23 bp, Artificial sequence
  Synthetic
cctcgtgaccaccctgacctgcg

SEQ ID NO: 9 - DNA, 23 bp, Artificial sequence
  Synthetic
cctcgtgaccaccctgacctacg

SEQ ID NO: 10 - DNA, 30 bp, Artificial sequence
  Synthetic
cttggggcccagactgagcacgtgatggca

SEQ ID NO: 11 - DNA, 30 bp, Artificial sequence; identical to SEQ ID NO: 10
  Synthetic

SEQ ID NO: 12 - DNA, 30 bp, Artificial sequence
  Synthetic
ccggtggcactgcggctggaggtgggggtt

SEQ ID NO: 13 - DNA, 30 bp, Artificial sequence
  Synthetic
aactggaacacaaagcatagactgcggggc

SEQ ID NO: 14 - DNA, 30 bp, Artificial sequence; identical to SEQ ID NO: 12
  Synthetic

SEQ ID NO: 15 - DNA, 30 bp, Artificial sequence; identical to SEQ ID NO: 12
  Synthetic

SEQ ID NO: 16 - DNA, 30 bp, Artificial sequence; identical to SEQ ID NO: 13
  Synthetic

SEQ ID NO: 17 - DNA, 30 bp, Artificial sequence; identical to SEQ ID NO: 13
  Synthetic

SEQ ID NO: 18 - DNA, 30 bp, Artificial sequence; identical to SEQ ID NO: 13
  Synthetic

SEQ ID NO: 19 - DNA, 30 bp, Artificial sequence; identical to SEQ ID NO: 13
  Synthetic

SEQ ID NO: 20 - DNA, 30 bp, Artificial sequence; identical to SEQ ID NO: 13
  Synthetic

SEQ ID NO: 21 - DNA, 30 bp, Artificial sequence; identical to SEQ ID NO: 13
  Synthetic

SEQ ID NO: 22 - DNA, 51 bp, Artificial sequence
  Synthetic
tatatcatggccgacaagcagaagaacggcatcaaggtgaacttcaagatc

SEQ ID NO: 23 - protein, 17 aa, Artificial sequence
  Synthetic
YIMADKQKNGIKVNFKI

SEQ ID NO: 24 - DNA, 30 bp, Artificial sequence
  Synthetic
gccgacaagcagaagaacggcatcaaggtg

SEQ ID NO: 25 - DNA, 30 bp, Artificial sequence; identical to SEQ ID NO: 24
  Synthetic

SEQ ID NO: 26 - DNA, 51 bp, Artificial sequence
  Synthetic
tccctggcccccaaggcgcagatcaaagagagcctgcgggcagagctcagg

SEQ ID NO: 27 - protein, 17 aa, Artificial sequence
  Synthetic
SLAPKAQIKESLRAELR

SEQ ID NO: 28 - DNA, 30 bp, Artificial sequence
  Synthetic
caaggcgcagatcaaagagagcctgcgggc

SEQ ID NO: 29 - DNA, 30 bp, Artificial sequence; identical to SEQ ID NO: 28
  Synthetic

SEQ ID NO: 30 - DNA, 33 bp, Artificial sequence
  Synthetic
cacaaacgtcgtatctccgcactcggcccaggc

SEQ ID NO: 31 - DNA, 23 bp, Artificial sequence
  Synthetic
tcagggtgagcatagactgccgg

SEQ ID NO: 32 - DNA, 23 bp, Artificial sequence
  Synthetic
cacccagactgagcacgtgctgg

SEQ ID NO: 33 - DNA, 23 bp, Artificial sequence
  Synthetic
caggaagctggagcacgtgaggg

SEQ ID NO: 34 - DNA, 23 bp, Artificial sequence
  Synthetic
tgcactgcggccggaggaggtgg

SEQ ID NO: 35 - DNA, 23 bp, Artificial sequence
  Synthetic
ggctctgcggctggagggggtgg

SEQ ID NO: 36 - DNA, 23 bp, Artificial sequence
  Synthetic
gtggctggaggtggaggtggggg

SEQ ID NO: 37 - DNA, 30 bp, Artificial sequence; identical to SEQ ID NO: 10
  Synthetic

SEQ ID NO: 38 - DNA, 30 bp, Artificial sequence; identical to SEQ ID NO: 12
  Synthetic

SEQ ID NO: 39 - DNA, 30 bp, Artificial sequence; identical to SEQ ID NO: 13
  Synthetic

SEQ ID NO: 40 - DNA, 23 bp, Artificial sequence
  Synthetic
cttgaccaatagccttgacaagg

SEQ ID NO: 41 - DNA, 23 bp, Artificial sequence
  Synthetic
gaacacaaagcatagactgcggg

SEQ ID NO: 42 - DNA, 23 bp, Artificial sequence
  Synthetic
ggcccagactgagcacgtgatgg

SEQ ID NO: 43 - DNA, 23 bp, Artificial sequence
  Synthetic
ggcccagactgagcacgtgatgg

SEQ ID NO: 44 - protein, 84 aa, Artificial sequence
  Synthetic
MTNLSDIIEKETGKQLVIQESILMLPEEVEEVIGNKPESDILVHTAYDESTDENVMLLTSDAPEYKPWALVIQDSNGENKIKML

SEQ ID NO: 45 - RNA, 93 nt, Artificial sequence
  Synthetic
guuuaagagcuaugcuggaaacagcauagcaaguuuaaauaaggcuaguccguuaucaacuugaaaaaguggcaccgagucggugcuuuuuuu

SEQ ID NO: 46 - RNA, 79 nt, Artificial sequence
  Synthetic
ggaaccauucaaaacagcauagcaaguuaaaauaaggcuaguccguuaucaacuugaaaaaguggcaccgagucggugc

SEQ ID NO: 47 - RNA, 60 nt, Artificial sequence
  Synthetic
uagcaaguuaaaauaaggcuaguccguuaucaacuugaaaaaguggcaccgagucggugc

SEQ ID NO: 48 - RNA, 64 nt, Artificial sequence
  Synthetic
agcauagcaaguuaaaauaaggcuaguccguuaucaacuugaaaaaguggcaccgagucggugc

SEQ ID NO: 49 - RNA, 70 nt, Artificial sequence
  Synthetic
caaaacagcauagcaaguuaaaauaaggcuaguccguuaucaacuugaaaaaguggcaccgagucggugc

SEQ ID NO: 50 - RNA, 45 nt, Artificial sequence
  Synthetic
uagcaaguuaaaauaaggcuaguccguuaucaacuugaaaaagug

SEQ ID NO: 51 - RNA, 32 nt, Artificial sequence
  Synthetic
uagcaaguuaaaauaaggcuaguccguuauca

SEQ ID NO: 52 - RNA, 26 nt, Artificial sequence
  Synthetic
uagcaaguuaaaauaaggcuaguccg

SEQ ID NO: 53 - RNA, 66 nt, Artificial sequence
  Synthetic
uucuugucguacuuauagaucgcuacguuauuucaauuuugaaaaucugaguccugggagugcgga

SEQ ID NO: 54 - protein, 609 aa, Artificial sequence
  Synthetic
MSGWESYYKTEGDEEAEEEQEENLEASGDYKYSGRDSLIFLVDASKAMFESQSEDELTPFDMSIQCIQSVYISKIISSDRDLLAVVFYGTEKDKNSVNFKNIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLMHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPMLKEACRAYGLKSGLKKQELLEALTKHFQD

SEQ ID NO: 55 - protein, 485 aa, Artificial sequence
  Synthetic
MVRSGNKAAVVLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISERHSIHWPCRLTIGSNLSIRIAAYKSILQERVKKTWTVVDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDMVAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAK

SEQ ID NO: 56 - RNA, 10 nt, Artificial sequence
  Synthetic
aauuuuugga

SEQ ID NO: 57 - protein, 83 aa, Artificial sequence
  Synthetic
GSVIDVSSQRVNVQRPLDALGNSLNSPVIIKLKGDREFRGVLKSFDLHMNLVLNDAEELEDGEVTRRLGTVLIRGDNIVYISP

SEQ ID NO: 58 - RNA, 25 nt, Artificial sequence
  Synthetic
gcgcacaugaggaucacccaugugc

SEQ ID NO: 59 - protein, 117 aa, Artificial sequence
  Synthetic
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 60 - RNA, 26 nt, Artificial sequence
  Synthetic
auaaggaguuuauauggaaacccuua

SEQ ID NO: 61 - protein, 128 aa, Artificial sequence
  Synthetic
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 62 - RNA, 19 nt, Artificial sequence
  Synthetic
cugaaugccugcgagcauc

SEQ ID NO: 63 - protein, 62 aa, Artificial sequence
  Synthetic
MKSIRCKNCNKLLFKADSFDHIEIRCPRCKRHIIMLNACEHPTEKHCGKREKITHSDETVRY

SEQ ID NO: 64 - protein, 1581 aa, Streptococcus pyogenes
PKKKRKVDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 65 - protein, 1581 aa, Streptococcus pyogenes
PKKKRKVDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 66 - DNA, 4743 bp, Artificial sequence
  Synthetic
ccaaagaagaagcggaaagtcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtc

SEQ ID NO: 67 - DNA, 4743 bp, Artificial sequence
  Synthetic
ccaaagaagaagcggaaagtcgacaagaaatactccattggactggccattggaaccaacagcgtcggatgggccgtgatcaccgacgagtataaagtcccctccaagaaattcaaggtgctgggcaataccgacagacattccatcaagaagaatctgatcggcgctctgctcttcgattccggcgagaccgccgaagctacaagactgaagagaacagctagaaggagatatacaagaaggaagaatagaatctgttacctccaagagatcttcagcaacgagatggccaaagtcgatgacagcttcttccacagactcgaagagagctttctcgtggaggaggacaagaagcacgagagacaccctatcttcggcaacatcgtggatgaggtcgcctatcatgagaaataccccaccatctaccatctgaggaagaaactcgtcgactccaccgataaagccgatctcagactgatctatctggctctggcccatatgatcaagtttaggggccactttctgattgagggcgacctcaaccccgacaactccgatgtggacaaactcttcatccagctggtccagacatacaaccagctgttcgaggagaaccctattaacgcctccggcgtggatgccaaggctattctgagcgccagactgtccaaatctagaaggctcgaaaacctcatcgctcaactgcccggcgagaaaaagaacggcctcttcggcaatctgattgccctctctctgggactgacccctaatttcaaatccaactttgatctggccgaggacgccaaactgcagctctccaaagacacatacgacgacgatctggacaatctgctcgctcagatcggagaccagtacgccgatctgtttctggccgccaagaacctctccgatgccattctgctgagcgacattctgagggtgaacacagaaatcaccaaggcccctctgtccgccagcatgatcaagaggtatgacgaacaccatcaagacctcacactgctgaaagccctcgtgagacagcaactccccgaaaaatacaaagagatcttttttgaccagagcaaaaatggctatgccggctatatcgatggcggcgctagccaagaggagttctacaaattcattaagcccattctggagaaaatggatggcacagaggaactgctggtgaagctgaatagggaggatctgctgagaaagcaaaggacattcgacaacggctccatcccccaccagattcatctgggcgagctccatgccattctgagaaggcaagaggacttctatcccttcctcaaagacaatagagagaaaatcgaaaagattctgaccttcagaatcccttattatgtcggccccctcgctagaggaaactctagattcgcttggatgacaagaaagtccgaggagacaatcaccccttggaactttgaggaagtggtggacaagggagccagcgcccagagcttcattgaaaggatgacaaattttgacaagaacctccccaacgagaaagtgctgcctaagcactctctgctgtacgagtacttcacagtctataatgagctgaccaaagtgaagtatgtcaccgaaggcatgaggaaacccgctttcctcagcggcgagcagaagaaggccatcgtcgatctgctgtttaagaccaatagaaaagtcaccgtcaaacagctgaaggaagattacttcaagaaaattgagtgcttcgactccgtggaaatcagcggcgtcgaggatagatttaacgcttctctgggcacataccatgatctgctgaagatcatcaaagacaaggattttctcgacaacgaagagaacgaggacatcctcgaggatatcgtgctgacactgaccctcttcgaggatagagaaatgatcgaggagaggctcaagacatatgcccacctcttcgacgacaaggtgatgaaacaactgaagagaagaagatacaccggctggggaagactctctagaaagctcatcaatggcattagggacaagcaaagcggaaagaccattctcgacttcctcaagtccgacggctttgccaataggaactttatgcagctcatccatgacgattctctgacattcaaggaggacatccagaaggcccaagtgagcggacaaggagattccctccatgaacatatcgctaacctcgccggatcccccgccattaaaaagggaatcctccaaacagtgaaggtcgtggatgagctggtcaaagtgatgggcagacacaaacccgagaacattgtcatcgagatggccagagagaaccagaccacccaaaaaggacagaagaactccagagaaaggatgaaaagaatcgaggaaggaatcaaggaactcggctcccagatcctcaaggagcatcccgtggagaatacccagctgcagaatgagaaactgtacctctactacctccagaatggaagggacatgtacgtcgaccaagaactcgacatcaacagactgagcgactacgatgtcgacgctatcgtgccccagagctttctgaaagacgactccatcgataacaaggtcctcacaagatccgacaagaacagaggcaagagcgacaacgtcccctccgaagaggtggtgaaaaagatgaagaactactggaggcagctgctgaacgccaaactcatcacccagaggaagttcgataatctgaccaaagccgaaagaggaggactgtccgaactggacaaagccggctttatcaagaggcagctggtggaaaccagacagatcaccaaacatgtcgcccaaattctggactctagaatgaacaccaagtacgacgaaaatgacaagctgattagagaagtgaaggtcatcaccctcaagagcaagctggtctccgattttagaaaggatttccaattctacaaggtcagagagatcaataattaccaccatgcccacgatgcctatctgaacgccgtggtgggaacagccctcatcaagaagtaccctaagctggaaagcgagttcgtgtatggagattataaagtctacgatgtgaggaagatgattgccaagtccgagcaagagatcggcaaggccaccgctaaatacttcttttattccaacatcatgaacttctttaaaaccgagatcacactcgctaatggcgagattaggaagagacctctgatcgagacaaacggcgagaccggcgagatcgtctgggacaagggcagagatttcgccaccgtgagaaaggtgctctccatgcctcaagtgaacatcgtgaaaaagaccgaggtgcagaccggcggcttctccaaggagtccattctgcccaaaaggaactccgacaagctcatcgctagaaagaaggattgggatcctaagaaatacggcggatttgactcccctacagtcgcttacagcgtgctcgtggtggccaaggtcgagaagggcaagtccaagaagctgaagtccgtgaaggagctgctgggaatcacaatcatggagaggtcctccttcgagaagaaccccatcgattttctggaggccaagggctacaaagaggtgaagaaagatctgatcattaagctgcccaaatattccctcttcgagctggagaacggaagaaaaaggatgctggcctccgctggcgaactgcagaagggaaacgagctcgctctccccagcaagtacgtcaacttcctctacctcgccagccactacgagaaactgaagggatcccccgaggacaatgagcagaagcagctcttcgtggagcagcacaagcattacctcgatgagatcatcgagcagatctccgaattcagcaagagggtcattctggctgacgccaacctcgataaggtcctcagcgcttacaacaagcacagagataagcccattagggagcaagccgaaaatatcatccatctgtttacactgacaaatctgggcgcccccgccgcttttaagtacttcgataccaccatcgatagaaagaggtacacctccacaaaagaggtgctggatgctaccctcatccatcagtccattaccggactctacgagaccagaattgatctctcccagctgggaggagatagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtc

SEQ ID NO: 68 - DNA, 4743 bp, Artificial sequence
  Synthetic
ccaaagaagaagcggaaagtcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtc

SEQ ID NO: 69 - DNA, 4743 bp, Artificial sequence
  Synthetic
cccaaaaagaagagaaaggtcgacaagaaatactccattggactggccattggaaccaacagcgtcggatgggccgtgatcaccgacgagtataaagtcccctccaagaaattcaaggtgctgggcaataccgacagacattccatcaagaagaatctgatcggcgctctgctcttcgattccggcgagaccgccgaagctacaagactgaagagaacagctagaaggagatatacaagaaggaagaatagaatctgttacctccaagagatcttcagcaacgagatggccaaagtcgatgacagcttcttccacagactcgaagagagctttctcgtggaggaggacaagaagcacgagagacaccctatcttcggcaacatcgtggatgaggtcgcctatcatgagaaataccccaccatctaccatctgaggaagaaactcgtcgactccaccgataaagccgatctcagactgatctatctggctctggcccatatgatcaagtttaggggccactttctgattgagggcgacctcaaccccgacaactccgatgtggacaaactcttcatccagctggtccagacatacaaccagctgttcgaggagaaccctattaacgcctccggcgtggatgccaaggctattctgagcgccagactgtccaaatctagaaggctcgaaaacctcatcgctcaactgcccggcgagaaaaagaacggcctcttcggcaatctgattgccctctctctgggactgacccctaatttcaaatccaactttgatctggccgaggacgccaaactgcagctctccaaagacacatacgacgacgatctggacaatctgctcgctcagatcggagaccagtacgccgatctgtttctggccgccaagaacctctccgatgccattctgctgagcgacattctgagggtgaacacagaaatcaccaaggcccctctgtccgccagcatgatcaagaggtatgacgaacaccatcaagacctcacactgctgaaagccctcgtgagacagcaactccccgaaaaatacaaagagatcttttttgaccagagcaaaaatggctatgccggctatatcgatggcggcgctagccaagaggagttctacaaattcattaagcccattctggagaaaatggatggcacagaggaactgctggtgaagctgaatagggaggatctgctgagaaagcaaaggacattcgacaacggctccatcccccaccagattcatctgggcgagctccatgccattctgagaaggcaagaggacttctatcccttcctcaaagacaatagagagaaaatcgaaaagattctgaccttcagaatcccttattatgtcggccccctcgctagaggaaactctagattcgcttggatgacaagaaagtccgaggagacaatcaccccttggaactttgaggaagtggtggacaagggagccagcgcccagagcttcattgaaaggatgacaaattttgacaagaacctccccaacgagaaagtgctgcctaagcactctctgctgtacgagtacttcacagtctataatgagctgaccaaagtgaagtatgtcaccgaaggcatgaggaaacccgctttcctcagcggcgagcagaagaaggccatcgtcgatctgctgtttaagaccaatagaaaagtcaccgtcaaacagctgaaggaagattacttcaagaaaattgagtgcttcgactccgtggaaatcagcggcgtcgaggatagatttaacgcttctctgggcacataccatgatctgctgaagatcatcaaagacaaggattttctcgacaacgaagagaacgaggacatcctcgaggatatcgtgctgacactgaccctcttcgaggatagagaaatgatcgaggagaggctcaagacatatgcccacctcttcgacgacaaggtgatgaaacaactgaagagaagaagatacaccggctggggaagactctctagaaagctcatcaatggcattagggacaagcaaagcggaaagaccattctcgacttcctcaagtccgacggctttgccaataggaactttatgcagctcatccatgacgattctctgacattcaaggaggacatccagaaggcccaagtgagcggacaaggagattccctccatgaacatatcgctaacctcgccggatcccccgccattaaaaagggaatcctccaaacagtgaaggtcgtggatgagctggtcaaagtgatgggcagacacaaacccgagaacattgtcatcgagatggccagagagaaccagaccacccaaaaaggacagaagaactccagagaaaggatgaaaagaatcgaggaaggaatcaaggaactcggctcccagatcctcaaggagcatcccgtggagaatacccagctgcagaatgagaaactgtacctctactacctccagaatggaagggacatgtacgtcgaccaagaactcgacatcaacagactgagcgactacgatgtcgaccacatcgtgccccagagctttctgaaagacgactccatcgataacaaggtcctcacaagatccgacaagaacagaggcaagagcgacaacgtcccctccgaagaggtggtgaaaaagatgaagaactactggaggcagctgctgaacgccaaactcatcacccagaggaagttcgataatctgaccaaagccgaaagaggaggactgtccgaactggacaaagccggctttatcaagaggcagctggtggaaaccagacagatcaccaaacatgtcgcccaaattctggactctagaatgaacaccaagtacgacgaaaatgacaagctgattagagaagtgaaggtcatcaccctcaagagcaagctggtctccgattttagaaaggatttccaattctacaaggtcagagagatcaataattaccaccatgcccacgatgcctatctgaacgccgtggtgggaacagccctcatcaagaagtaccctaagctggaaagcgagttcgtgtatggagattataaagtctacgatgtgaggaagatgattgccaagtccgagcaagagatcggcaaggccaccgctaaatacttcttttattccaacatcatgaacttctttaaaaccgagatcacactcgctaatggcgagattaggaagagacctctgatcgagacaaacggcgagaccggcgagatcgtctgggacaagggcagagatttcgccaccgtgagaaaggtgctctccatgcctcaagtgaacatcgtgaaaaagaccgaggtgcagaccggcggcttctccaaggagtccattctgcccaaaaggaactccgacaagctcatcgctagaaagaaggattgggatcctaagaaatacggcggatttgactcccctacagtcgcttacagcgtgctcgtggtggccaaggtcgagaagggcaagtccaagaagctgaagtccgtgaaggagctgctgggaatcacaatcatggagaggtcctccttcgagaagaaccccatcgattttctggaggccaagggctacaaagaggtgaagaaagatctgatcattaagctgcccaaatattccctcttcgagctggagaacggaagaaaaaggatgctggcctccgctggcgaactgcagaagggaaacgagctcgctctccccagcaagtacgtcaacttcctctacctcgccagccactacgagaaactgaagggatcccccgaggacaatgagcagaagcagctcttcgtggagcagcacaagcattacctcgatgagatcatcgagcagatctccgaattcagcaagagggtcattctggctgacgccaacctcgataaggtcctcagcgcttacaacaagcacagagataagcccattagggagcaagccgaaaatatcatccatctgtttacactgacaaatctgggcgcccccgccgcttttaagtacttcgataccaccatcgatagaaagaggtacacctccacaaaagaggtgctggatgctaccctcatccatcagtccattaccggactctacgagaccagaattgatctctcccagctgggaggagattccggcggcagcggaggaagcggcggatccaccaatctgtccgacattatcgagaaggagaccggaaaacaactcgtgatccaagagtccatcctcatgctgcccgaggaagtcgaggaagtgatcggaaataagcccgagagcgatattctggtgcataccgcttacgacgagagcaccgacgaaaatgtcatgctgctgacctccgatgctcccgagtacaaaccttgggctctcgtcattcaagacagcaacggagagaacaagattaagatgctcagcggcggaagcggaggcagcggcggctccacaaatctgtccgatatcatcgaaaaggagaccggcaagcaactggtgatccaagagagcattctgatgctccccgaagaggtggaagaggtgatcggcaataaacccgagagcgacattctggtgcacacagcctacgatgagtccaccgatgagaacgtgatgctgctgaccagcgatgcccccgaatataagccttgggctctggtgattcaagactccaatggagagaataagatcaaaatgctctccggcggaagcaaaagaaccgccgatggcagcgaatttgagcctaaaaaaaagaggaaggtg

SEQ ID NO: 70 - DNA, 129 bp, Artificial sequence
  Synthetic
  n (1)..(20): a or g or c or t/u, unknown, or other
  misc_feature (1)..(20): n is a, c, g, or t
nnnnnnnnnnnnnnnnnnnngttttagagctagaaatagcaagttaaaataaggctagtccgttatcaacttgaaaaagtggcaccgagtcggtgcgcgcacatgaggatcacccatgtgctttttttg

SEQ ID NO: 71 - DNA, 157 bp, Artificial sequence
  Synthetic
gttttagagctagaaatagcaagttaaaataaggctagtccgttatcaacttgaaaaagtggcaccgagtcggtgcgggagcacatgaggatcacccatgtgccacgagcgacatgaggatcacccatgtcgctcgtgttccctttttttctccgct

SEQ ID NO: 72 - protein, 340 aa, Artificial sequence
  Synthetic
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGLELKTPLGDTTHTSPPCPAPELLGGPMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 73 - DNA, 1041 bp, Artificial sequence
  Synthetic
cccaagaagaagcggaaagtgatggatagcctgctgatgaaccggagaaagttcctgtatcagtttaagaatgtgcgctgggcaaagggcaggcgcgagacctacctgtgctatgtggtgaagcggagagattccgccacatccttctctctggactttggctacctgcggaacaagaatggctgccacgtggagctgctgttcctgagatacatctctgactgggatctggacccaggcaggtgttatcgcgtgacctggttcacaagctggtccccctgctacgattgtgcaaggcacgtggcagactttctgaggggaaacccaaatctgtccctgcggatcttcaccgccagactgtatttttgcgaggataggaaggcagagccagagggactgaggcgcctgcacagggccggcgtgcagatcgccatcatgaccttcaaggactacttttattgttggaacaccttcgtggagaatcacgagcggaccttcaaggcctgggagggactgcacgagaactccgtgcggctgtctagacagctgcggagaatcctgctgcctctgtacgaggtggacgatctgagggatgccttccgcaccctgggactggagctgaagacacccctgggcgacaccacacacacctctccaccttgcccagcaccagagctgctgggaggccctatggccagcaacttcacacagtttgtgctggtggataatggaggaaccggcgacgtgacagtggcaccatctaactttgccaatggcatcgccgagtggatcagctccaactctcggagccaggcctataaggtgacctgtagcgtgcggcagtctagcgcccagaatagaaagtatacaatcaaggtggaggtgcctaagggcgcctggagatcctacctgaacatggagctgaccatcccaatctttgccacaaattctgattgcgagctgatcgtgaaggccatgcagggcctgctgaaggacggcaaccctatcccaagcgccatcgccgccaatagcggaatctac

SEQ ID NO: 74 - DNA, 1131 bp, Artificial sequence
  Synthetic
cccaagaagaagcggaaagtgtcctcagagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccaataccagatgtagcatcacatggtttctgagctggtccccttgcggagagtgtagcagggccatcaccgagttcctgtccagatatccacacgtgacactgtttatctacatcgccaggctgtatcaccacgcagacccaaggaataggcagggcctgcgcgatctgatcagctccggcgtgaccatccagatcatgacagagcaggagtccggctactgctggcggaacttcgtgaattattctcctagcaacgaggcccactggcctaggtacccacacctgtgggtgcgcctgtacgtgctggagctgtattgcatcatcctgggcctgcccccttgtctgaatatcctgcggagaaagcagccccagctgaccttctttacaatcgccctgcagtcttgtcactatcagaggctgccaccccacatcctgtgggccacaggcctgaaggagctgaagacacccctgggcgacaccacacacacctctccaccttgcccagcaccagagctgctgggaggccctatggccagcaacttcacacagtttgtgctggtggataatggaggaaccggcgacgtgacagtggcaccatctaactttgccaatggcatcgccgagtggatcagctccaactctcggagccaggcctataaggtgacctgtagcgtgcggcagtctagcgcccagaatagaaagtatacaatcaaggtggaggtgcctaagggcgcctggagatcctacctgaacatggagctgaccatcccaatctttgccacaaattctgattgcgagctgatcgtgaaggccatgcagggcctgctgaaggacggcaaccctatcccaagcgccatcgccgccaatagcggaatctac

SEQ ID NO: 75 - DNA, 1131 bp, Artificial sequence
  Synthetic
cccaagaagaagcggaaagtgagctccgaaaccggacccgtggccgtggaccctacactgaggagaaggatcgagccccacgagtttgaggtgttcttcgaccccagagaactgaggaaggagacatgtctgctgtatgagatcaactggggcggaagacactccatctggaggcacacaagccagaacaccaacaagcacgtcgaggtgaacttcatcgagaagttcaccaccgagaggtacttctgccccaacacaagatgctccatcacatggtttctgagctggagcccttgcggcgaatgctccagagccatcaccgagtttctgtctagatacccccacgtgacactgtttatctacatcgctagactgtaccaccatgccgatcccagaaacagacaaggactgagggatctgatctccagcggcgtgaccatccagatcatgaccgagcaagagtccggctactgctggaggaacttcgtgaactactcccctagcaacgaggcccactggcccagataccctcatctgtgggtgagactgtacgtgctcgagctgtactgtatcattctgggactgcctccttgtctgaacattctgagaaggaagcagccccagctgaccttcttcaccatcgctctgcagagctgccactaccagaggctgcctccccacattctgtgggccaccggactgaaggagctgaagacacccctgggcgacaccacacacacctctccaccttgcccagcaccagagctgctgggaggccctatggccagcaacttcacacagtttgtgctggtggataatggaggaaccggcgacgtgacagtggcaccatctaactttgccaatggcatcgccgagtggatcagctccaactctcggagccaggcctataaggtgacctgtagcgtgcggcagtctagcgcccagaatagaaagtatacaatcaaggtggaggtgcctaagggcgcctggagatcctacctgaacatggagctgaccatcccaatctttgccacaaattctgattgcgagctgatcgtgaaggccatgcagggcctgctgaaggacggcaaccctatcccaagcgccatcgccgccaatagcggaatctac

SEQ ID NO: 76 - protein, 198 aa, Artificial sequence [sequence omitted]

SEQ ID NO: 77 - DNA, 594 bp, Artificial sequence
  Synthetic
atggacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccatgaaagaactttcaaagcctgggaagggctgcatgaaaattcagttcgtctctccagacagcttcggcgcatccttttgcccctgtatgaggttgatgacttacgagacgcatttcgtactttgggactt

SEQ ID NO: 78 - DNA, 594 bp, Artificial sequence
  Synthetic
atggatagcctgctgatgaaccggagaaagttcctgtatcagtttaagaatgtgcgctgggcaaagggcaggcgcgagacctacctgtgctatgtggtgaagcggagagattccgccacatccttctctctggactttggctacctgcggaacaagaatggctgccacgtggagctgctgttcctgagatacatctctgactgggatctggacccaggcaggtgttatcgcgtgacctggttcacaagctggtccccctgctacgattgtgcaaggcacgtggcagactttctgaggggaaacccaaatctgtccctgcggatcttcaccgccagactgtatttttgcgaggataggaaggcagagccagagggactgaggcgcctgcacagggccggcgtgcagatcgccatcatgaccttcaaggactacttttattgttggaacaccttcgtggagaatcacgagcggaccttcaaggcctgggagggactgcacgagaactccgtgcggctgtctagacagctgcggagaatcctgctgcctctgtacgaggtggacgatctgagggatgccttccgcaccctgggactg

SEQ ID NO: 79 - protein, 198 aa, Artificial sequence
  Synthetic
MDSLLMNRRKFLYQFKNVRWAKGRRETYLCYVVKRRDAATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGL

SEQ ID NO: 80 - DNA, 594 bp, Artificial sequence
  Synthetic
atggacagcctcttgatgaaccggaggaagtttctttaccaattcaaaaatgtccgctgggctaagggtcggcgtgagacctacctgtgctacgtagtgaagaggcgtgacgccgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccatgaaagaactttcaaagcctgggaagggctgcatgaaaattcagttcgtctctccagacagcttcggcgcatccttttgcccctgtatgaggttgatgacttacgagacgcatttcgtactttgggactt

SEQ ID NO: 81 - DNA, 594 bp, Artificial sequence
  Synthetic
atggatagcctgctgatgaaccggagaaagttcctgtatcagtttaagaatgtgcgctgggcaaagggcaggcgcgagacctacctgtgctatgtggtgaagcggagagatgccgccacatccttctctctggactttggctacctgcggaacaagaatggctgccacgtggagctgctgttcctgagatacatctctgactgggatctggacccaggcaggtgttatcgcgtgacctggttcacaagctggtccccctgctacgattgtgcaaggcacgtggcagactttctgaggggaaacccaaatctgtccctgcggatcttcaccgccagactgtatttttgcgaggataggaaggcagagccagagggactgaggcgcctgcacagggccggcgtgcagatcgccatcatgaccttcaaggactacttttattgttggaacaccttcgtggagaatcacgagcggaccttcaaggcctgggagggactgcacgagaactccgtgcggctgtctagacagctgcggagaatcctgctgcctctgtacgaggtggacgatctgagggatgccttccgcaccctgggactg

SEQ ID NO: 82 - RNA, 122 nt, Artificial sequence
  Synthetic
  n (1)..(20): a or g or c or t/u, unknown, or other
  misc_feature (1)..(19): n is a, c, g, or u
nnnnnnnnnnnnnnnnnnnguuuuagagcuagaaauagcaaguuaaaauaaggcuaguccguuaucaacuugaaaaaguggcaccgagucggugcgggagcacaugaggaucacccaugugc

SEQ ID NO: 83 - RNA, 149 nt, Artificial sequence
  Synthetic
  n (1)..(20): a or g or c or t/u, unknown, or other
  misc_feature (1)..(19): n is a, c, g, or u
nnnnnnnnnnnnnnnnnnnguuuuagagcuagaaauagcaaguuaaaauaaggcuaguccguuaucaacuugaaaaaguggcaccgagucggugcgggagcacaugaggaucacccaugugccacgagcgacaugaggaucacccaugu

SEQ ID NO: 84 - DNA, 24 bp, Artificial sequence
  Synthetic
  n (5)..(24): a or g or c or t/u, unknown, or other
  misc_feature (5)..(24): n is a, c, g, or t
ctagnnnnnnnnnnnnnnnnnnnn

SEQ ID NO: 85 - DNA, 24 bp, Artificial sequence
  Synthetic
  misc_feature (5)..(24): n is a, c, g, or t
aaacnnnnnnnnnnnnnnnnnnnn

SEQ ID NO: 86 - DNA, 20 bp, Artificial sequence
  Synthetic
gcagcagtgaaagagttctt

SEQ ID NO: 87 - DNA, 20 bp, Artificial sequence
  Synthetic
cagccagctgtctcagttta

SEQ ID NO: 88 - DNA, 20 bp, Artificial sequence
  Synthetic
aaacgtcgtatctccgcact

SEQ ID NO: 89 - DNA, 20 bp, Artificial sequence
  Synthetic
cgtatctccgcactcggccc

SEQ ID NO: 90 - DNA, 21 bp, Artificial sequence
  Synthetic
ttggcgaaatggcggaaaacc

SEQ ID NO: 91 - DNA, 22 bp, Artificial sequence
  Synthetic
caccgacggataccacctgctg

SEQ ID NO: 92 - DNA, 23 bp, Artificial sequence
  Synthetic
cctggctgagctaactgtgacag

SEQ ID NO: 93 - DNA, 27 bp, Artificial sequence
  Synthetic
gtcaaactgtgcgtatgacatcatcag

SEQ ID NO: 94 - DNA, 23 bp, Artificial sequence
  Synthetic
gcatgcatttgtaggcttgatgc

SEQ ID NO: 95 - DNA, 23 bp, Artificial sequence
  Synthetic
gcccctgtctaggaaaagctgtc

SEQ ID NO: 96 - DNA, 23 bp, Artificial sequence
  Synthetic
ctgggtggaaggaagggaggaag

SEQ ID NO: 97 - DNA, 23 bp, Artificial sequence
  Synthetic
tcaacccgaacggagacacacac

SEQ ID NO: 98 - DNA, 25 bp, Artificial sequence
  Synthetic
ctgtttgccttaggagaggccagag

SEQ ID NO: 99 - DNA, 26 bp, Artificial sequence
  Synthetic
ctctgaacacaagcctttctccaggg

SEQ ID NO: 100 - DNA, 24 bp, Artificial sequence
  Synthetic
gacctggagaagcatgaaccagtc

SEQ ID NO: 101 - DNA, 25 bp, Artificial sequence
  Synthetic
catggtgtgcctgtcactgtacttg

SEQ ID NO: 102 - DNA, 23 bp, Artificial sequence
  Synthetic
gaggtccaaggaggcctatgcag

SEQ ID NO: 103 - DNA, 29 bp, Artificial sequence
  Synthetic
gggaaggagacttagtgagacttgaaacc

SEQ ID NO: 104 - DNA, 24 bp, Artificial sequence
  Synthetic
ctgagcgcacatcccttgtctctc

SEQ ID NO: 105 - DNA, 23 bp, Artificial sequence
  Synthetic
ctgctactggagcacaccccaag

SEQ ID NO: 106 - DNA, 25 bp, Artificial sequence
  Synthetic
catagctggggctgaagatccctag

SEQ ID NO: 107 - DNA, 26 bp, Artificial sequence
  Synthetic
ctcctcggagtcctcaagtatcactg

SEQ ID NO: 108 - DNA, 23 bp, Artificial sequence
  Synthetic
gtgcttgggttgctttggcaatg

SEQ ID NO: 109 - DNA, 24 bp, Artificial sequence
  Synthetic
gttgctttggcaatggaggcattg

SEQ ID NO: 110 - DNA, 27 bp, Artificial sequence
  Synthetic
gtgaagaactccaggggcaatctgaag

SEQ ID NO: 111 - DNA, 25 bp, Artificial sequence
  Synthetic
caccacctcttccatctgccttgtc

SEQ ID NO: 112 - DNA, 23 bp, Artificial sequence
  Synthetic
cttcaaggaggacggcaacatcc

SEQ ID NO: 113 - DNA, 22 bp, Artificial sequence
  Synthetic
tgttctgctggtagtggtcggc

SEQ ID NO: 114 - DNA, 26 bp, Artificial sequence
  Synthetic
cggcatcaaggtgaacttcaagatcc

SEQ ID NO: 115 - DNA, 22 bp, Artificial sequence
  Synthetic
ctcgttggggtctttgctcagg

SEQ ID NO: 116 - DNA, 20 bp, Artificial sequence
  Synthetic
cgcaggtcagggtggtcacg

SEQ ID NO: 117 - DNA, 20 bp, Artificial sequence
  Synthetic
caagcagaagaacggcatca

SEQ ID NO: 118 - DNA, 20 bp, Artificial sequence
  Synthetic
gaacacaaagcatagactgc

SEQ ID NO: 119 - DNA, 20 bp, Artificial sequence
  Synthetic
ggcccagactgagcacgtga

SEQ ID NO: 120 - DNA, 20 bp, Artificial sequence
  Synthetic
ggcactgcggctggaggtgg

SEQ ID NO: 121 - DNA, 20 bp, Artificial sequence
  Synthetic
cgcagatcaaagagagcctg

SEQ ID NO: 122 - protein, 4 aa, Artificial sequence
  Synthetic
GSGS

SEQ ID NO: 123 - protein, 5 aa, Artificial sequence
  Synthetic
GSGRA

SEQ ID NO: 124 - protein, 10 aa, Artificial sequence
  Synthetic
GSGSGSGSGS

SEQ ID NO: 125 - protein, 15 aa, Artificial sequence
  Synthetic
GGGGSGGGGSGGGGS

SEQ ID NO: 126 - protein, 25 aa, Artificial sequence
  Synthetic
ELKTPLGDTTHTSPPCPAPELLGGP

SEQ ID NO: 127 - DNA, 23 bp, Artificial sequence
  Synthetic
gaacacaaagcatagactgcggg

SEQ ID NO: 128 - DNA, 23 bp, Artificial sequence
  Synthetic
tcagggtgagcatagactgccgg

SEQ ID NO: 129 - DNA, 23 bp, Artificial sequence
  Synthetic
ggcccagactgagcacgtgatgg

SEQ ID NO: 130 - DNA, 23 bp, Artificial sequence
  Synthetic
cacccagactgagcacgtgctgg

SEQ ID NO: 131 - DNA, 23 bp, Artificial sequence
  Synthetic
gacacagaccgggcacgtgaggg

SEQ ID NO: 132 - DNA, 23 bp, Artificial sequence
  Synthetic
caggaagctggagcacgtgaggg

SEQ ID NO: 133 - DNA, 23 bp, Artificial sequence
  Synthetic
ggcactgcggctggaggtggggg

SEQ ID NO: 134 - DNA, 23 bp, Artificial sequence
  Synthetic
tgcactgcggccggaggaggtgg

SEQ ID NO: 135 - DNA, 23 bp, Artificial sequence
  Synthetic
ggctctgcggctggagggggtgg

SEQ ID NO: 136 - DNA, 23 bp, Artificial sequence
  Synthetic
gtggctggaggtggaggtggggg

SEQ ID NO: 137 - protein, 168 aa, Anolis carolinensis
MEPEAFQRNFDPREFPECTLLLYEIHWDNNTSRNWCTNKPGLHAEENFLQIFNEKIDIKQDTPCSITWFLSWSPCYPCSQAIIKFLEAHPNVSLEIKAARLYMHQIDCNKEGLRNLGRNRVSIMNLPDYRHCWTTFVVPRGANEDYWPQDFLPAITNYSRELDSILQD

SEQ ID NO: 138 - DNA, 504 bp, Artificial sequence
  Synthetic
atggagccggaggcttttcagcgcaactttgaccctcgggaatttcctgaatgtacactcctcttgtatgagatccactgggacaataacacatctagaaattggtgtacgaataagcctgggctccacgctgaggagaatttcttgcagatatttaatgagaaaattgacattaaacaggatacgccgtgctctataacatggttcctttcttggagcccctgttacccttgtagccaagcaataataaaattcttggaggcacacccgaatgtcagtctggagattaaggctgcgcggctgtatatgcatcaaatagactgtaacaaggagggactcagaaatctgggccggaatcgagtgtcaataatgaacctgcctgattataggcattgctggactacgtttgttgtgccaaggggagcaaacgaagattactggccacaagactttctgcctgcgatcacaaattactcccgagaactcgactccatactgcaggat

SEQ ID NO: 139 - protein, 229 aa, Myotis lucifugus
MASDAGSSAGDPTLRRRIEPWDFEAIFDPRELRKEACLLYEIKWGPCHKIWRHSGKNTTRHVEVNFIEKITSERQFCSSTSCSIIWFLSWSPCWECSKAITEFLRQRPGVTLVIYVARLYHHMDEQNRQGLRDLIKSGVTIQIMTTPEYDYCWRNFVNYPPGKDTHCPMYPPLWMKLYALELHCIILSLPPCLMISRRCQKQLTWYRLNLQNCHYQQIPPHILLATAWI

SEQ ID NO: 140 - DNA, 687 bp, Artificial sequence
  Synthetic
atggcttcagacgcaggctcctccgcaggggatcctactttgaggcgaaggatcgaaccatgggacttcgaagcaattttcgatcctcgagagctgaggaaagaagcctgtctgttgtacgaaattaagtggggaccctgtcacaaaatatggcggcattctggcaaaaataccactagacacgtcgaggttaactttatcgaaaaaatcacaagcgagcggcaattctgttcttccacatcatgttccattatctggttcctttcatggagcccatgttgggagtgctctaaagcaataaccgagtttctcaggcagagacctggagtaactctcgtaatctacgtcgcccggctctaccaccacatggatgagcaaaatcgacaggggcttcgggatctcattaaaagtggtgtcacgatacaaattatgacgactccagagtacgattactgctggcggaactttgtgaactacccaccgggcaaggatacccactgtcctatgtatccacccctgtggatgaaactttacgcactcgagctgcattgtatcattctctcccttccaccgtgtctcatgatctcacgcaggtgtcaaaagcagttgacttggtacagattgaaccttcaaaattgccactatcaacagattccgcctcatattttgctggcaactgcgtggata

SEQ ID NO: 141 - protein, 348 aa, Myotis lucifugus
PKKKRKVMMDSLLMKQKKFLYHFKNLRWAKGRHETYLCYVVKQRNSATSCSLDFGYLRNKSGCHVEVLFLRYISTWDLDPRHCYRITWFTSWSPCYDCARHVADFLSAYPNLSLRIFAARLYFCEERNAEPEGLRRLHRAGAQIAIMTFKDYFYCWNTFVENRKTTFKAWEGLHENSVRLARRLRRILLPLYEVDDLRDAFRMLGLELKTPLGDTTHTSPPCPAPELLGGPMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 142 - DNA, 1044 bp, Artificial sequence
  Synthetic
cccaagaagaagcggaaagtgatgatggacagccttctgatgaagcaaaagaaatttctttatcacttcaaaaatctgcgctgggctaaggggaggcacgagacgtatctctgttatgtagtgaaacaaagaaatagtgccacgtcttgttcccttgatttcggttatctccgaaacaagagcggatgccacgttgaagttctgtttttgaggtacatcagcacgtgggacctcgacccgagacattgctaccgaataacttggttcacatcctggagcccctgttatgactgcgctcgccacgtagccgattttcttagtgcttaccctaacctttcactcaggattttcgccgcacgactgtatttctgcgaggaacgcaatgctgagcctgaaggtctccggaggctccaccgagccggggctcaaatagccattatgacatttaaggattacttttattgttggaatacgtttgtagagaaccgaaagaccacatttaaggcgtgggaaggtctgcatgagaatagtgtcagacttgcgaggaggctgcggaggatcctcttgcccctctatgaagtagatgatctccgcgatgcgttcaggatgttgggacttgagctgaagacacccctgggcgacaccacacacacctctccaccttgcccagcaccagagctgctgggaggccctatggccagcaacttcacacagtttgtgctggtggataatggaggaaccggcgacgtgacagtggcaccatctaactttgccaatggcatcgccgagtggatcagctccaactctcggagccaggcctataaggtgacctgtagcgtgcggcagtctagcgcccagaatagaaagtatacaatcaaggtggaggtgcctaagggcgcctggagatcctacctgaacatggagctgaccatcccaatctttgccacaaattctgattgcgagctgatcgtgaaggccatgcagggcctgctgaaggacggcaaccctatcccaagcgccatcgccgccaatagcggaatctac

SEQ ID NO: 143 - protein, 317 aa, Artificial sequence
  Synthetic
PKKKRKVMEPEAFQRNFDPREFPECTLLLYEIHWDNNTSRNWCTNKPGLHAEENFLQIFNEKIDIKQDTPCSITWFLSWSPCYPCSQAIIKFLEAHPNVSLEIKAARLYMHQIDCNKEGLRNLGRNRVSIMNLPDYRHCWTTFVVPRGANEDYWPQDFLPAITNYSRELDSILQDELKTPLGDTTHTSPPCPAPELLGGPMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 144 - DNA, 951 bp, Artificial sequence
  Synthetic
cccaagaagaagcggaaagtgatggagccggaggcttttcagcgcaactttgaccctcgggaatttcctgaatgtacactcctcttgtatgagatccactgggacaataacacatctagaaattggtgtacgaataagcctgggctccacgctgaggagaatttcttgcagatatttaatgagaaaattgacattaaacaggatacgccgtgctctataacatggttcctttcttggagcccctgttacccttgtagccaagcaataataaaattcttggaggcacacccgaatgtcagtctggagattaaggctgcgcggctgtatatgcatcaaatagactgtaacaaggagggactcagaaatctgggccggaatcgagtgtcaataatgaacctgcctgattataggcattgctggactacgtttgttgtgccaaggggagcaaacgaagattactggccacaagactttctgcctgcgatcacaaattactcccgagaactcgactccatactgcaggatgagctgaagacacccctgggcgacaccacacacacctctccaccttgcccagcaccagagctgctgggaggccctatggccagcaacttcacacagtttgtgctggtggataatggaggaaccggcgacgtgacagtggcaccatctaactttgccaatggcatcgccgagtggatcagctccaactctcggagccaggcctataaggtgacctgtagcgtgcggcagtctagcgcccagaatagaaagtatacaatcaaggtggaggtgcctaagggcgcctggagatcctacctgaacatggagctgaccatcccaatctttgccacaaattctgattgcgagctgatcgtgaaggccatgcagggcctgctgaaggacggcaaccctatcccaagcgccatcgccgccaatagcggaatctac

SEQ ID NO: 145 - protein, 347 aa, Myotis brandtii
PKKKRKVMDSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGNPNLSLRIFTARLYFCEDYKAEPEGLRRLHRAGAQIAIMTFKDYFYCWNTFVENRERTFRAWEGLHENSVRLSRQLRRILLPLYEVDDLRDAFRTLGLELKTPLGDTTHTSPPCPAPELLGGPMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 146 - DNA, 1041 bp, Artificial sequence
  Synthetic
cccaagaagaagcggaaagtgatggactctctgctgatgaagcagaggaagtttctgtaccacttcaagaacgtgagatgggccaagggcagacacgaaacctatctgtgctacgtggtgaagaggagggacagcgccacctccttttctctggatttcggccacctcagaaacaagtccggctgccacgtggagctgctgtttctgaggtacatcagcgattgggatctggaccccggaagatgctatagagtgacatggttcaccagctggagcccttgctacgactgcgccagacacgtggccgactttctgagaggcaaccccaatctgtctctgagaatcttcaccgctagactgtacttctgcgaggactacaaggccgagcccgaaggactgagaaggctgcatagagccggcgcccagatcgccatcatgaccttcaaggactacttctactgctggaacaccttcgtggaaaatagagagagaacctttagagcttgggagggcctccatgagaactccgtgaggctgtctagacaactgaggagaattctgctccctctgtatgaggtcgatgatctgagagacgccttcagaacactgggactggagctgaagacacccctgggcgacaccacacacacctctccaccttgcccagcaccagagctgctgggaggccctatggccagcaacttcacacagtttgtgctggtggataatggaggaaccggcgacgtgacagtggcaccatctaactttgccaatggcatcgccgagtggatcagctccaactctcggagccaggcctataaggtgacctgtagcgtgcggcagtctagcgcccagaatagaaagtatacaatcaaggtggaggtgcctaagggcgcctggagatcctacctgaacatggagctgaccatcccaatctttgccacaaattctgattgcgagctgatcgtgaaggccatgcagggcctgctgaaggacggcaaccctatcccaagcgccatcgccgccaatagcggaatctac

SEQ ID NO: 147 - DNA, 3582 bp, Artificial sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacccgtctcacaggcggatcgatccaaggtcgggcaggaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcgggagcacatgaggatcacccatgtgccacgagcgacatgaggatcacccatgtcgctcgtgttccctttttttctccgctgagcgtactgagacgccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgatcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcgatctttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcaggactcttcctttttcaatattattgaagcatttatcagggttattgtctcatgagcggatacatatttgaatgtatttagaaaaataaacaaataggggttccgcgcacatttccccgaaaagtgccac

SEQ ID NO: 148 - DNA, 23 bp, Artificial sequence
  Synthetic
cgcagatcaaagagagcctgcgg

SEQ ID NO: 149 - DNA, 23 bp, Artificial sequence
  Synthetic
cttgaccaatagccttgacaagg

SEQ ID NO: 150 - DNA, 28 bp, Artificial sequence
  Synthetic
cgatgtggacgctatcgtgcctcagagc

SEQ ID NO: 151 - DNA, 18 bp, Artificial sequence
  Synthetic
tagtcggacagccggttg

SEQ ID NO: 152 - DNA, 170 bp, Artificial sequence
  Synthetic
gaacacaaagcatagactgcgttttagagctagaaatagcaagttaaaataaggctagtccgttatcaacttgaaaaagtggcaccgagtcggtgcgggagcacatgaggatcacccatgtgccacgagcgacatgaggatcacccatgtcgctcgtgttcccttttttt

SEQ ID NO: 153 - DNA, 103 bp, Artificial sequence
  Synthetic
gaacacaaagcatagactgcgttttagagctagaaatagcaagttaaaataaggctagtccgttatcaacttgaaaaagtggcaccgagtcggtgcttttttt

SEQ ID NO: 154 - DNA, 170 bp, Artificial sequence
  Synthetic
gcactaccagagctaactcagttttagagctagaaatagcaagttaaaataaggctagtccgttatcaacttgaaaaagtggcaccgagtcggtgcgggagcacatgaggatcacccatgtgccacgagcgacatgaggatcacccatgtcgctcgtgttcccttttttt

(19) United States
(12) Patent Application Publication
(10) Pub. No.: US 2022/0290134 A1
(43) Pub. Date: Sep. 15, 2022

(54) Highly Efficient DNA Base Editors Mediated By RNA-Aptamer Recruitment For Targeted Genome Modification And Uses Thereof

(71) Applicant: Rutgers,The State University of New Jersey, New Brunswick, NJ (US)

(72) Inventors: Shengkan Jin, Belle Mead, NJ (US); Juan-Carlos Collantes, Princeton, NJ (US)

(73) Assignee: Rutgers,The State University of New Jersey, New Brunswick, NJ (US)

(21) Appl. No.: 17/753,659

(22) PCT Filed: Sep. 16, 2020
(86) PCT No.: PCT/US20/51048

§ 371 Date: Mar. 10, 2022

Related U.S. Application Data
(60) Provisional application No. 62/901,584, filed on Sep. 17, 2019.

(51) Int. Cl. C12N 15/11; C12N 9/22; C12N 15/62; C12N 9/78; C12N 15/115; C12N 15/90; C07K 14/47; A61K 35/22

(52) U.S. Cl. CPC C12N 15/11; C12N 9/22; C12N 15/62; C12N 9/78; C12Y 305/04005; C12N 15/115; C12N 15/907; C07K 14/47; A61K 35/22; C12N 2310/20; C12N 2310/16; C12N 2800/80; C12N 2310/3519; C07K 2319/85; C07K 2319/09; C12N 2310/531

(57) ABSTRACT

The present invention discloses a system for targeted gene editing and related uses. Also disclosed are related cells.

### CROSS REFERENCE TO RELATED APPLICATION

This application claims priority to U.S. Provisional Application No. 62/901,584 filed on Sep. 17, 2019, the disclosures of which is incorporated herein by reference.

### FIELD OF THE INVENTION

This invention relates to a system for targeted genome modification and uses thereof.

### BACKGROUND OF THE INVENTION

Gene editing technologies, such as zinc finger nucleases (ZFN), transcription activator-like effector nucleases (TALEN) or clustered regularly interspaced short palindromic repeats (CRISPR) systems, provide powerful tools for biotechnology and biomedical research in general. They have also generated hope for the systemic development of targeted therapies for genetic diseases, cancer, viral infections, and beyond. However, gene-editing technologies have important limitations that need to be addressed before its widespread use in clinical practice. First, conventional gene editing systems rely on the generation of DNA double strand breaks (DSBs) at target sites, which could potentially have deleterious consequences, especially if unintended off-target activity is high (1, 2). Although the development of strategies such as paired nickases (3), catalytically inactive Cas9 fused to dimeric nucleases (4, 5) or high-fidelity CRISPR systems (6, 7) are thought to mitigate these adverse effects, due to limitation of detection methods to accurately assess on- and off-target mutagenesis the actual adverse effects of gene editing interventions may be underestimated. It was recently shown that DSBs generated by CRISPR systems induce previously unnoticed deletions and rearrangements that span several kilobases at on-target sites (8). Likewise, insertional mutagenesis has been observed in experiments using purified Cas9/sgRNA ribonucleoprotein complexes (RNP) (9), a method thought to enhance targeting specificity. Second, in order to introduce precise modifications, such as point mutations, it is often necessary that the target cells undergo homology dependent DNA double strand break repair (HDR) (10, 11). Somatic cells, in particular terminally differentiated somatic cells, however, do not have high HDR activity and instead utilize the error prone non-homologous end joining (NHEJ) pathway (12). These findings highlight the needs for new gene editing systems for developing safe and efficacious therapeutics.

### SUMMARY OF INVENTION

This invention addresses the needs mentioned above in a number of aspects.

In one aspect, the invention provides a system comprising: (i) a sequence-targeting component or a polynucleotide encoding the same; (ii) an RNA scaffold, or a polynucleotide (such as DNA) encoding the same; and (iii) a first effector fusion protein, or a polynucleotide encoding the same. The sequence-targeting component comprises a target fusion protein having (a) a sequence-targeting protein and (b) a first uracil DNA glycosylase (UNG) inhibitor peptide (UGI). The RNA scaffold comprises (a) a nucleic acid-targeting motif comprising a guide RNA sequence that is complementary to a target nucleic acid sequence, (b) an RNA motif (e.g., a CRISPR motif described herein) capable of binding to the sequence-targeting protein, and (c) a first recruiting RNA motif. The first effector fusion protein comprises (a) a first RNA binding domain capable of binding to the first recruiting RNA motif, (b) a linker, and (c) an effector domain. The first effector fusion protein or the effector domain has an enzymatic activity, such as cytosine deamination activity or adenosine deamination activity. In one embodiment, an exemplary system is a called Cas-RNA aptamer mediated C to U Reversion (CasRCure or CRC) system. Additional exemplary systems include Second Generation CRC systems CRC_AID (^{A}CRCnu, ^{A}CRCnu.2) and CRC_APOBEC1 (^{A1}CRCnu., ^{A1}CRCnu.2) as described herein (u indicating presence of UGI in the system).

In a system of this invention, the target fusion protein can comprise one, two, or more UGIs. The RNA scaffold can comprise one, two or more recruiting RNA motifs. Accordingly, the target fusion protein can further comprise two or more UGIs (e.g., a second UGI). The RNA scaffold can further comprise two or more recruiting RNA motifs (e.g., a second recruiting RNA motif). Preferably, one, more, or all the coding sequences are codon optimized. For example, one or more of the polynucleotides encoding the sequence-targeting protein, the first UGI, the second UGI, the RNA binding domain, and the effector domain are optimized for expression in eukaryotic cells (e.g., plant cells, insect cells, or mammalian cells). Each of the sequence-targeting component and the first effector fusion protein can have a nuclear localization signal (NLS). For example, the sequence-targeting component or the first effector fusion protein comprises one or more NLSes. In one embodiment, the sequence-targeting component comprises two NLSes. In that case, the two NLSes can be at the N-terminus and C-terminus of the sequence-targeting component respectively as shown in FIG. 9C.

In the system described above, the sequence-targeting protein can be a CRISPR protein. The sequence-targeting protein does not have a nuclease activity. Examples of the sequence-targeting protein include the sequence of dCas9 or nCas9 of a species selected from the group consisting of Streptococcus pyogenes, Streptococcus agalactiae, Staphylococcus aureus, Streptococcus thermophilus, Streptococcus thermophilus, Neisseria meningitidis, and Treponema denticola.

In the above mentioned RNA scaffold, the first recruiting RNA motif and the first RNA binding domain can be a pair selected from the group consisting of: (1) a telomerase Ku binding motif and Ku protein or an RNA-binding section thereof, (2) a telomerase Sm7 binding motif and Sm7 protein or an RNA-binding section thereof, (3) a MS2 phage operator stem-loop and MS2 coat protein (MCP) or an RNA-binding section thereof, (4) a PP7 phage operator stem-loop and PP7 coat protein (PCP) or an RNA-binding section thereof, (5) a SfMu phage Com stem-loop and Com RNA binding protein or an RNA-binding section thereof, (6) a chemically modified version of the above mentioned aptamers and their corresponding aptamer ligand or an RNA-binding section thereof and (7) a non-natural RNA aptamer and corresponding aptamer ligand or an RNA-binding section thereof.

The effector fusion protein can have various suitable enzymatic activities. In one embodiment, the effector can have a cytidine deamination activity, such as a wild type or genetically engineered version of AID, CDA, APOBEC1, APOBEC3A, APOBEC3B, APOBEC3C, APOBEC3D, APOBEC3F, or other APOBEC family enzymes of a species selected from the group consisting of human, rat, mouse, bat, naked mole rat, elephant, chicken, lizard, giant tortoise, coelacanth, and other vertebrate species. In another embodiment, the effector can have an adenine deamination activity, such as a wild type or genetically engineered version of ADA, ADAR family enzymes, or tRNA adenosine deaminases of a species selected from the group consisting of bacteria, yeast, human, rat, mouse, bat, naked mole rat, elephant, chicken, lizard, giant tortoise, coelacanth, and other vertebrate species. The linker sequence can be 0 to 100 (e.g., 1-100, 5-80, 10-50, and 20-30) amino acid residues in length.

Also provided are an isolated nucleic acid encoding one or more of components (i)-(iii) of the system described above, an expression vector comprising the nucleic acid, or a host cell comprising the nucleic acid.

In a second aspect, the invention provides a method of site-specific modification of a target DNA. The method includes contacting the target nucleic acid with components (i)-(iii) of the system described above. The target nucleic acid can be in a cell. The target nucleic acid can be RNA, an extrachromosomal DNA, or a genomic DNA on a chromosome. The cell can be selected from the group consisting of: an archaeal cell, a bacterial cell, a eukaryotic cell, a eukaryotic single-cell organism, a somatic cell, a germ cell, a stem cell, a plant cell, an algal cell, an animal cell, an invertebrate cell, a vertebrate cell, a fish cell, a frog cell, a bird cell, a mammalian cell, a pig cell, a cow cell, a goat cell, a sheep cell, a rodent cell, a rat cell, a mouse cell, a horse cell, a non-human primate cell, and a human cell. The cell can be in or derived from a human or non-human subject. The human or non-human subject can have a genetic mutation of a gene. In some embodiments, the subject has a disorder caused by the genetic mutation or is at risk of having the disorder. In that case, the site-specific modification corrects the genetic mutation or inactivates the expression of the gene. In other embodiments, the subject has a pathogen or is at risk of exposing to the pathogen, and the site-specific modification inactivates a gene of the pathogen.

Accordingly, this invention also provides a genetically engineered cell obtained according to the method described above. The cell can be selected from the group consisting of a stem cell, an immune cell, and a lymphocyte. Examples of the stem cell include embryonic stem cells, ES-like stem cells, fetal stem cells, adult stem cells, pluripotent stem cells, induced pluripotent stem cells, multipotent stem cells, oligopotent stem cells, unipotent stem cells, and others described herein. Examples of the immune cell include a T cell, a B cell, an NK cell, a macrophage, a mixture thereof, and others described herein. Also provided is a pharmaceutical composition comprising an effective amount of the cell and a pharmaceutically acceptable carrier.

The invention further provides a kit containing the system described above or one or more components thereof. The system can further contain one or more components selected from the group consisting of a reagent for reconstitution and/or dilution and a reagent for introducing nucleic acid or polypeptide into a host cell.

The details of one or more embodiments of the invention are set forth in the description below. Other features, objectives, and advantages of the invention will be apparent from the description and from the claims.

### BRIEF DESCRIPTION OF THE DRAWINGS

FIGS. 1A, 1B, 1C, 1D, 1E and 1F are a set of diagrams showing a CRC System and proof-of-principle in prokaryotic cells. A. Components of the CRC platform, from left to right: 1 sequence targeting component dCas9 or nCas9_{D10A}, 2 Chimeric RNA scaffold containing a guide RNA motif (for sequence targeting; 2.1), CRISPR motif (for Cas9 binding; 2.2), and recruiting RNA aptamer motif (for recruiting effector-RNA binding protein fusion; 2.3), and 3 fusion protein consisting of effector cytidine deaminase (3.1)—RNA-aptamer proteins ligand (3.2). B. Schematic of CRC complex at the target sequence: Cas9 binds to CRISPR RNA, the recruiting RNA aptamer recruits the effector module, forming an active CRC complex capable of editing target C residues (shaded) on the unpaired DNA within the CRISPR R-loop. PAM sequences are underlined. C. RRDR Cluster I region (SEQ ID NO: 2 (nucleic acid sequence) and SEQ ID NO: 3 (corresponding amino acid sequence)) of E. coli's rpoB gene, with PAM sequences underlined, critical cytosines shaded in gray boxes. Arrows represent gRNA targeting sites. Shaded in gray is the RRDR protein sequence. D. Representative pictures showing surviving bacterial colonies after treatment with CRC targeted with the indicated gRNAs expressing one MS2 copy (1×MS2). E. Quantification of survival fraction cells from similar experiments shown in D. Bars show standard deviation of the mean from 3 independent experiments. F. Representative sequencing results from untreated cells (top, SEQ ID NO: 4) and ^{A}CRCd treatment with rpoB_TS4_1_×MS2 gRNA (bottom, SEQ ID NO: 5). Target position are indicated with black asterisk. This C1592>T mutation results in S531F change in protein sequence, a mutation known to induce rifampicin resistance (23, 24).

FIGS. 2A, 2B, and 2C are a set of diagrams showing engineering of CRC modules to enhance base editing efficiency in bacterial cells. A. Effect of replacing Cas9 nickases (nCas9H_{840A} or nCas9_{D10A}) with dCas9 and increasing the number of recruiting motifs from 1×MS2 to 2×MS2. B. Effect of linker length variation in effector module. L4, L5, L10, L12 and L25 are linker peptides consisting of 4, 5, 10, 12 and 25 amino acids, respectively. C. Comparison of AID (^{A}CRC_{D10A}), APOEC3G (^{A3G}CRC_{D10A}) and APOBEC1 (^{A1}CRC_{D10A}) as effectors. The figures show representative results from 3 independent experiments.

FIGS. 3A, 3B, 3C, 3D, 3E, 3F, and 3G are a set of diagrams and photographs showing the effect of CRC on correcting a target mutation and on global mutagenesis in human cells. A. Non-fluorescent EGFP (nfEGFP) target region (SEQ ID NO: 6). A→G loss-of-function mutation at chromophore sequence (underlined in black). One gRNA targeting the non-template strand (NT1) is shown as an arrow, a PAM sequence is underlined, and the target cytosine is shaded in gray. Corresponding protein sequence (SEQ ID NO: 7) shown shaded in gray. B. Effect on editing extrachromosomal gene. HEK 293 cells were transiently transfected with target DNA containing the nfEGFP mutant together with ^{A}CRCnu, BE4max or BE3 components and nfEGFP_NT1 gRNA. Panels show representative sections of plates under a fluorescence microscope after the indicated treatments. C. Flow cytometry analysis of cells expressing extrachromosomal nfEGFP gene treated with ^{A}CRCnu, BE4max and BE3 targeted with nfEGFP_NT1. D. Flow cytometry analysis of HEK 293 cell stably expressing the non-fluorescent EGFP mutant gene (nf2.16 cells) treated with ^{A}CRCnu, BE4max and BE3 guided by nfEGFP_NT1 gRNA. E. Sequencing of sorted fluorescent cells. *G→A conversion of the target nucleotide (top: SEQ ID NO: 8; bottom: SEQ ID NO: 9). Note that base editing occurs on complementary strand. F. Whole exome sequencing and comparison of SNPs of nf2.16 cells treated with ^{A}CRCnu/nfEGFP_NT-1, ^{A}CRCnu/Scramble or untreated. Genomic DNA was isolated and subjected to whole exome sequencing. The figure shows the global distribution of single nucleotide polymorphisms of the three treatments compared to the human reference genome (hg38), including AID signature mutations C→T/G→A. Statistical analysis showed no significant difference in all SNP categories. G. Comparison of occurrences of C>T and G>A events at “AID motif” sequences (WRCH/DGYW; dark gray bars) versus “non-motif” (NNCN/NGNN; light gray bars). Mutations on CpG sites were not counted to avoid overestimation due to higher mutation rates at these sites. p values were calculated using Chi-square test. NT1: nfEGFP_NT1 gRNA (NT=targeted to the non-template strand). Error bars represent standard deviation of the mean from three independent experiments. All gRNAs used in CRC treatments express 2 MS2 aptamers for effector recruitment.

FIGS. 4A, 4B, 4C, 4D, 4E, and 4F are a set of diagrams showing that CRC system efficiently edits endogenous sites (SEQ ID NOs: 10-15) in the human genome. HEK293 cells were treated with ^{A}CRCnu or ^{A1}CRCnu and the indicated gRNAs. A-C. Quantification of single nucleotide mutations induced by ^{A}CRCnu at the indicated loci. D-F. Quantification of single nucleotide mutations induced by ^{A1}CRCnu at the indicated loci. Treatments were analyzed by high throughput sequencing to quantify frequency of mutations induced by the systems tested in this set of experiments. gRNA target sequences are shaded in gray. All gRNAs used in these experiments express 2 MS2 aptamers for effector recruitment.

FIGS. 5A, 5B, 5C, 5D, 5E, and 5F are a set of diagrams showing that optimization of CRC constructs leads to enhanced base editing efficiency. Cells were treated with the indicated base editing system and targeted to Site 2 (SEQ ID NOs: 16, 18, and 20). High throughput sequencing analysis reveals enhanced efficiency after targeting Site 2 with ^{A}CRCnu.2 (A) and ^{A1}CRCnu.2 (C), reaching a comparable efficiency to BE4max (E). Cells were treated with the corresponding systems with scramble gRNA (B, D, F, SEQ ID NOs: 17, 19, and 21). Target sequence is shaded in gray. All gRNAs used in CRC treatments express 2 MS2 aptamers for effector recruitment.

FIGS. 6A, 6B, 6C, 6D, 6E, 6F, 6G, and 6H are a set of diagrams and of photographs showing that CRC mediates efficient knockout in a GFP reporter and an endogenous site in human cells. A. Schematic representation of the EGFP region (SEQ ID NO: 22) targeted in these experiments. One gRNA (arrow) was designed to induce a stop codon at residue Q157 (EGFP_TS1); PAM sequence is underlined. Corresponding protein sequence (SEQ ID NO: 23) is shown shaded in gray. B. HEK293 cells expressing an EGFP transgene were treated with ^{A}CRCnu.2 and EGFP_TS1. Panels show representative sections of plates under fluorescence microscope. C. Cells from a similar experiment shown in B were subjected to flow cytometry analysis to quantify GFP loss. Error bars represent standard deviation of the mean from at least three independent experiments. D-E. High throughput sequencing analysis of an EGFP reporter cells treated with ^{A}CRCnu.2 and EGFP_TS1 (D) (SEQ ID NO: 24), or untreated (E) (SEQ ID NO: 25). F. Schematic representation of the endogenous PDCD1 locus region (SEQ ID NO: 26) targeted in these experiments. One gRNA (arrow) was designed to induce a stop codon at residue Q133 (PDCD1_TS1); PAM sequence. Corresponding protein sequence (SEQ ID NO: 27) is shown shaded in gray. G-H. High throughput sequencing analysis of the endogenous PDCD1 locus treated with ^{A}CRCnu.2 and PDCD1_TS1 gRNA (G) (SEQ ID NO: 28), or untreated (H) (SEQ ID NO: 29). TS: targeted to the template strand. All gRNAs used in these experiments express 2 MS2 aptamers for effector recruitment.

FIGS. 7A, 7B, and 7C are a set of diagrams showing bacterial expression constructs. A-C. Schematic representation of constructs used in bacterial experiments, including DNA targeting module encoding for Cas9 variants dCas9, nCas9_{D10A} or nCas9_{H840A} (A; component (1) in FIG. 1A); gRNA/recruiting module containing one or two RNA aptamer motifs (B, top and bottom, respectively; component (2) in FIG. 1A); and effector module, encoding for fusion proteins AID_MCP, APOBEC1_MCP or APOBEC3G_MCP (C; component (3) in FIG. 1A).

FIGS. 8A and 8B are a set of diagrams showing mutation distribution in rpoB gene sequence (SEQ ID NO: 30) targeted E. coli cells. Mutation distribution of clones selected on rifampicin plates after treatment. All experiments use TS4 gRNA for comparison. A. Side by side comparison of editing outcomes after treatment with CRC systems with different Cas9 variants (i.e., ^{A}CRC_{d} with dCas9, ^{A}CRC_{H840A} with nCas9_{H840A} and ^{A}CRC_{D10A} with nCas9_{D10A}). B. Side by side comparison of editing outcomes after treatment with CRC systems with different effector proteins (i.e., ^{A1}CRC_{D10A} with APOBEC1 and ^{A3G}CRC_{D10A} with APOBEC3G). RpoB gene from individual clones was PCR amplified and sequenced for genotyping. Numbers represent percentage of clones with a given genotype.

FIGS. 9A, 9B, and 9C are a set of diagrams showing mammalian expression constructs. A. Schematic representation of first-generation ^{A}CRCnu multicistronic construct expressing AID_L25_MCP fusion protein and nCas9_{D10A}-UGI. The two modules are separated by a self-cleaving 2A, and their expression is driven by a CMV promoter. B. gRNA_2×MS2 constructs express from a U6 promoter. C. Second-generation ^{A}CRCnu.2 system follows a similar architecture as to first-generation, with key differences: optimization of codons, enhanced nuclear localization for Cas9-UGI module and increased number of UGI copies. NLS: nuclear localization signal; Effector: AID, APOBEC1; L25: 25 amino acid flexible linker; 2A: self-cleavable 2A peptide.

FIGS. 10A, 10B, 10C, 10D, 10E, and 10F are a set of diagrams showing frequency of indel formation after treatment with ^{A}CRCnu and ^{A1}CRCnu targeting site 2, site 3 and site 4. Histograms showing indel analysis of the experiments show in FIG. 4, with the indicated CRC system and targeting gRNA. A-C show indels induced by ^{A}CRCnu targeted to Site 2, Site 3 and Site 4. D-F show indels induced by ^{A1}CRCnu targeted to the same sites. The gRNA target sites are indicated as black lines. Note that indels tend to accumulate with higher frequency at the gRNA target site.

FIG. 11 is a set of diagrams showing high throughput sequencing analysis of selected off-target sites (homologous sites) after ^{A}CRCnu and ^{A}CRCnu.2 treatments targeting Site 2, Site 3, or Site 4. Analysis of known S. pyogenes Cas9 off-target sites (31, 32) for Site 2: S2O2; Site 3: S3O1, S3O2 and S3O3; and Site 4: S4O1, S4O2 and S4O4 (SEQ ID NOs: 31-36). Off-target sequences are summarized in Table S5.

FIGS. 12A, 12B, 12C, 12D, 12E, and 12F are a set of diagrams showing frequencies and distributions of indel formation after treatment with ^{A}CRCnu.2, ^{A1}CRCnu.2, or BE4max targeting Site 2. Histograms quantifying indel frequencies of the experiments shown in FIG. 5. Cells were treated with the indicated systems and gRNAs and subjected to high throughput sequencing. The gRNA target sequences are indicated as a black line.

FIGS. 13A, 13B, 13C, and 13D are a set of diagrams showing high throughput sequencing analysis of ^{A}CRCnu.2 targeted to Site 3 and Site 4. HEK293 cells were treated with ^{A}CRCnu.2 and the indicated gRNAs, targeted to Site 3 (A) (SEQ ID NO: 37), and Site 4 (C) (SEQ ID NO: 38). Untreated counterparts are shown in B for Site 3 and D for Site 4. Samples were then analyzed by high throughput sequencing to quantify frequency of mutations induced by the system. Target sequence is shaded in gray. All gRNAs used in these experiments express 2 MS2 aptamers for effector recruitment.

FIGS. 14A, 14B, 14C, and 14D are a set of diagrams frequencies and distributions of indel formation after treatment with ^{A}CRCnu.2 at Site 3 and Site 4. Histograms quantifying indel frequencies of the experiments shown in FIGS. 13A-D. Cells were treated with the indicated systems and gRNAs and subjected to high throughput sequencing. The gRNA target site indicated as a black line.

FIGS. 15A and 15B are a set of diagrams showing frequency of indel formation after treatment with ^{A}CRCnu.2 targeting EGFP transgene. Histograms showing indel analysis of the experiments shown in FIGS. 5A-F, where ^{A}CRCnu.2 was targeted to EGFP using gRNAs TS1 (A). Untreated counterparts are shown in B. The gRNA target sequences are indicated as a black line.

FIGS. 16A, 16B, 16C, and 16D are a set of diagrams showing: (A) Single nucleotide polymorphisms (SNPs) across region of site 2 (SEQ ID NO: 39) targeted with Site 2 gRNA and second-generation rat ^{A1}CRCnu.2; (B) SNPs across region of Site 2 targeted with Site 2 gRNA and second-generation lizard (Anolis carolinensis)^{LizardA1}CRCnu.2; (C) SNPs across region of Site 2 targeted with Site 2 gRNA and second-generation Bat (Myotis lucifugus)^{BatA1}CRCnu.2 and (D) SNPs across region of Site 2 in untreated cells.

FIG. 17 is a diagram showing comparison of C to T conversion rates at a human fetal hemoglobin promoter locus (HBF) (SEQ ID NO: 40) in K562 cells by ^{LizardA1}CRCnu.2 (labelled as lizard Apobec 1), rat ^{A1}CRCnu.2 (labelled as rat Apobec 1), BE4max (labelled as BE4), and ^{LizardA1}CRCnu.2 (labelled as lizard AID) systems. PAM motif is AGG at the 3′ end.

FIG. 18 is a diagram showing comparison of C to T conversion rates at the Site 2 locus (SEQ ID NO: 41) in HEK293 cells by ^{LizardA1}CRCnu.2 (labelled as lizard Apobec 1) and rat ^{A1}CRCnu.2 (labelled as rat Apobec 1) systems. PAM motif is GGG at the 3′ end.

FIG. 19 is a diagram showing comparison of C to T conversion rates at the Site 3 locus (SEQ ID NO: 42) in HEK293 cells by ^{LizardA}CRCnu.2 (labelled as lizard AID) and human ^{A}CRCnu.2 (labelled as human AID) systems. PAM motif is TGG at the 3′ end.

FIG. 20 is a diagram showing comparison of C to T conversion rates at the Site 3 locus (SEQ ID NO: 43) in HEK293 cells by ^{BatA}CRCnu.2 (labelled as bat AID) and human ^{A}CRCnu.2 (labelled as human AID) systems.

FIG. 21 is a diagram showing C to T conversion using a catalytically dead Cas9 (dCas9) version of the ^{A}CRCnu.2 construct at Site2 which contains two target Cs (C¹ and C²) within the editing window. All experiments were performed with the ^{A}CRCnu.2 version of the base editing system and included both the original nCas9 version (^{A}CRCnu.2) and a derived dCas9 version (^{A}CRCnu.2_dCas9). As a control the experiment included a sgRNA lacking the aptamer component of the system (^{A}CRCnu.2_dCas9_MS2less), the lack of the MS2 element of the system should lead to loss of editing due to a failure to recruit the deaminase through its fusion to MCP. A scrambled non-targeting sgRNA (^{A}CRCnu.2_dCas9_scrambled) was also included as a negative control. Data is shown as the percentage of T sequenced at the indicated target C residue as measured by Sanger sequencing. Error bars represent the standard deviation of the mean from 3 replicate experiments.

### DETAILED DESCRIPTION OF THE INVENTION

This invention relates to a new system for targeted genome modification and uses thereof. This invention is based, at least in part, on a novel RNA-aptamer mediated base editing system.

Conventional nuclease-dependent precise genome editing usually requires introduction of DNA double strand breaks (DSBs) and activation of the homology dependent repair (HDR) pathway. However, DSBs often carry oncogenic liability and HDR activity is low in somatic cells. Recently a base editing (BE) system has been developed in which a cytidine (or adenine) deaminase effector is recruited to the target DNA sequence through a direct fusion to a nuclease deficient Cas9 protein. BE changes a target base pair without requiring DSB or HDR.

An alternative and modularly designed base editing system was also developed. This system recruits the effector deaminase through the RNA component of the CRISPR complex. This system, named CasRCure (CRC), contains a modified gRNA with a re-programmable RNA-aptamer at the 3′ end, which recruits the cognate aptamer ligand fused to an effector (such as a deaminase effector). Using this system, targeted nucleotide modification was achieved with high precision in prokaryotic cells and eukaryotic cells including mammalian cells. See WO2018129129 and WO2017011721. As disclosed herein, a new, second generation CRC base editors CRC system with increased efficacy was tested and further improved in mammalian cells. The second generation of CRC base editors including one or all of the following features. First, the Cas9 protein contains one, two, or more than two UGIs; second, the Cas9-UGI protein has at least two nuclear localization signal peptides (NLS); and three, both the Cas9-UGI and the effector proteins are codon optimized for expression in the targeted host cells (e.g. mammalian cells). The second generation system/platform exhibits higher efficacy and specificity than the previously disclosed first generation CRC system. Importantly, various effector orthologs from different species were constructed with the Second Generation CRC configuration. Surprisingly, some Second Generation CRC with certain orthologs such as lizard orthologs exhibit unique features different from all previously documented base editors. For example, they have wider activity window allowing modification of nucleotides close to the PAM motif than the canonical activity window of position 3-9. With a modular design that fully separates the nucleic acid modification module from the nucleic acid recognition module as well as other advantages disclosed herein, the CRC base editing platform provides an alternative to recruitment of the effector through fusion to or direct interaction with the sequence-targeting protein, which could not effectively separate sequence-targeting function from nucleic acid modification function. Devoid of the requirements of DNA DSB and HDR, the new CRC system provides powerful tools for genetic engineering and for therapeutic development.

### Gene Editing Platform

One aspect of this invention provides a gene-editing platform, which overcomes the aforementioned limitations of conventional nuclease and DSB dependent genome-engineering and gene-editing technologies. The platform has three functional components: (1) a nuclease defective CRISPR/Cas-based module engineered for sequence targeting; (2) an RNA scaffold-based module for guiding the platform to a target sequence as well as for recruitment of a correction module; and (3) a non-nuclease DNA/RNA modifying enzyme as an effector correction module, such as cytidine deaminases (e.g., activation-induced cytidine deaminase, AID). Together, the CasRcure system allows specific DNA/RNA sequence anchoring, flexible and modular recruitment of effector DNA/RNA modifying enzymes to specific sequences, and eliciting cellular pathways that are active in somatic cells for correcting genetic information, in particular point mutation.

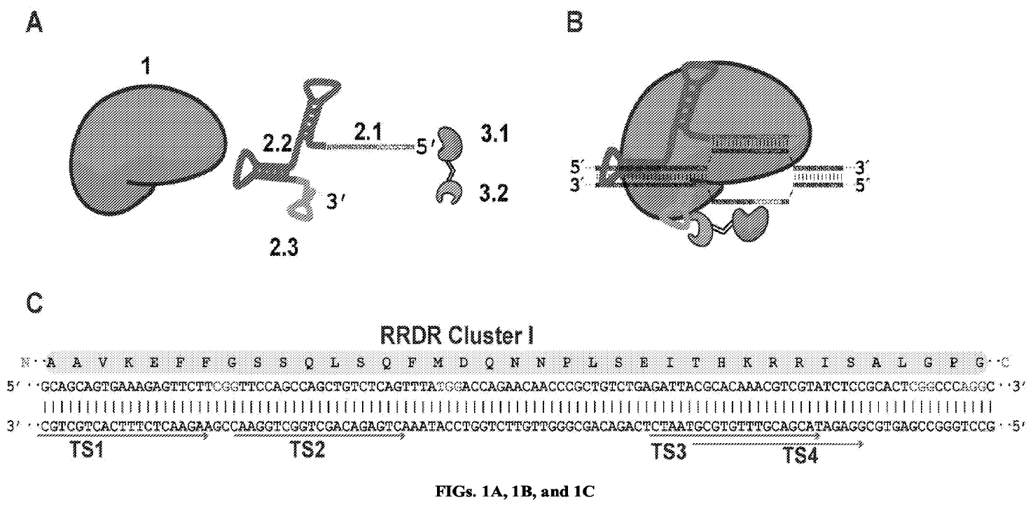

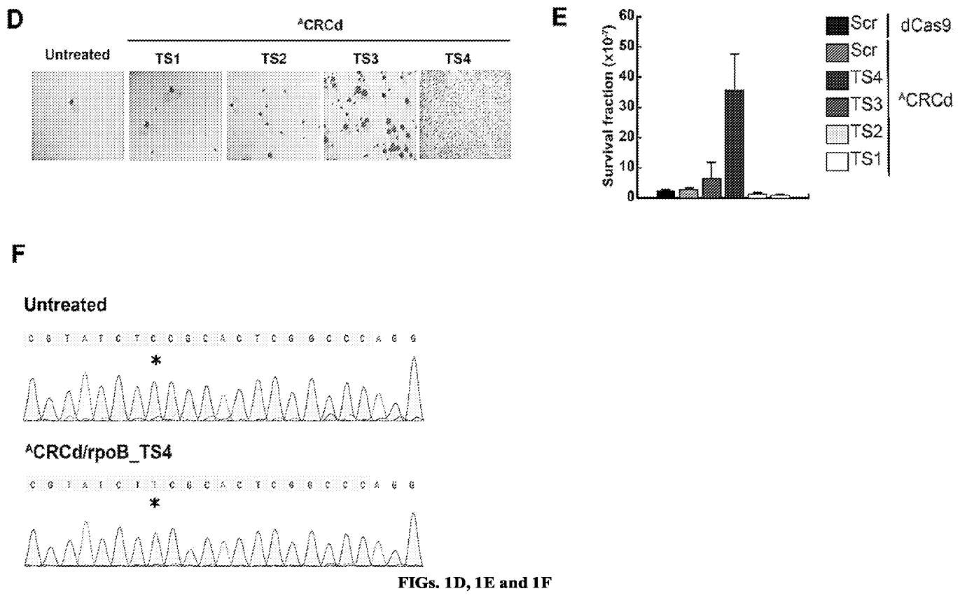

Illustrated in FIGS. 1A and 1B are schematics of an exemplary CasRcure system. The system includes three structural and functional components: (1) a sequence targeting module (e.g., a dCas9 protein); (2) an RNA scaffold for sequence recognition and for effector recruitment (an chimeric RNA molecule that contains a guide RNA (gRNA) motif, a CRISPR RNA motif, and a recruiting RNA motif), and (3) an effector (a non-nuclease DNA modifying enzyme such as AID fused to a small protein that binds to the recruiting RNA motif). More specifically as shown in FIG. 1A, the components of the CRC platform include: a sequence targeting component 1 (such as dCas9 or nCas9_{D10A}); a chimeric RNA scaffold 2 containing a guide RNA motif 2.1 (for sequence targeting), a CRISPR motif 2.2 (for Cas9 binding), and a recruiting RNA aptamer motif 2.3 (for recruiting effector-RNA binding protein fusion), and a fusion protein 3 comprising an effector 3.1 (e.g., cytidine deaminase) fused to an RNA aptamer ligand 3.2. FIG. 1B shows a schematic of the CRC complex at the target sequence: Cas9 binds to CRISPR RNA, the recruiting RNA aptamer recruits the effector module, forming an active CRC complex capable of editing target C residues on the unpaired DNA within the CRISPR R-loop, also known as protospacer. The three components can be constructed in a single expression vector or in multiple separate expression vectors. The totality and the combination of the three specific components constitute the enabling of the technologic platform. Although FIG. 1B shows three components of the RNA scaffold in a particular 3′ to 5′ order, the components can also be arranged in different orders when required, such as optimization for different Cas protein variants.

As disclosed herein, there is a number of clear distinctions between recruitment mechanisms: the RNA scaffold mediated recruitment system (the CRC system) versus the direct fusion of Cas9 to effector protein system (the BE system). The modular design of the CRC system allows for flexible system engineering. Modules are interchangeable and many combinations of different modules can be achieved by simply swapping the nucleotide sequence of the recruiting RNA aptamer and the cognate ligand. Recruitment of an effector by direct fusion or direct interaction with the protein component of the sequence-targeting unit, on the other hand, always requires a re-engineering of a new fusion protein, which is technically more difficult with a less predictable outcome. Furthermore, RNA scaffold mediated recruitment likely facilitates oligomerization of effector proteins, while direct fusion would preclude the formation of oligomers due to steric hindrance.

Because of its relative ease of use and scalability, the CRISPR/Cas based gene system is poised to dominate the therapeutic landscape, making it an attractive gene editing technology to develop novel applications with therapeutic value. As disclosed herein, the second-generation CRC base editor system takes advantages of certain aspects of the CRISPR/Cas system. To overcome the limitations associated with requirement of DSB and HDR for conventional CRISPR/Cas gene editing system, an elegant gene editing method called base editing (BE) has been developed exploiting the DNA targeting ability of Cas9 devoid of its nuclease activity, combined with the DNA editing capabilities of APOBEC-1, an enzyme member of the APOBEC family of DNA/RNA cytidine deaminases (13). By directly fusing the deaminase effector to the nuclease deficient Cas9 protein, these tools, called base editors, can introduce targeted point mutations in genomic DNA (13) or RNA (14) without generating DSBs or requiring HDR activity. In essence, the BE system utilizes a nuclease deficient CRISPR/Cas9 complex as a DNA targeting machinery, in which the mutant Cas9 serves as an anchor to recruit cytidine or adenine deaminase through a direct protein-protein fusion.

The CRC system, on the other hand, takes a different approach. More specifically, in the CRC system, the RNA component of the CRISPR/Cas9 complex serves as an anchor for effector recruitment by including an RNA aptamer into the RNA molecule. In turn, the RNA aptamer recruits an effector fused to the RNA aptamer ligand. Comparing to the recruitment by direct protein fusion or other recruiting approaches by the protein component, the RNA aptamer mediated effector recruitment mechanism has a number of distinct features potentially advantageous both for system engineering and for achieving better functionality. For example, it has a modular design in which the nucleic acid sequence targeting function and effector function reside in different molecules, making it possible to independently reprogram the functional modules and to multiplex the system. The re-programming of CRC system requires only the change of RNA aptamer sequence in gRNA and swap of the cognate RNA aptamer ligand fusing effector. It does not require re-engineering of an individual functional Cas9 fusion protein. In addition, the fusion effector is smaller in size which could potentially allow more efficient oligomerization of the functional effector. Moreover, as CRC does not require generation of a Cas9 fusion protein, which further increases the gene/transcription size of Cas9, CRC system could potentially be constructed in a way that is more efficient for packaging and delivery by viral vectors.

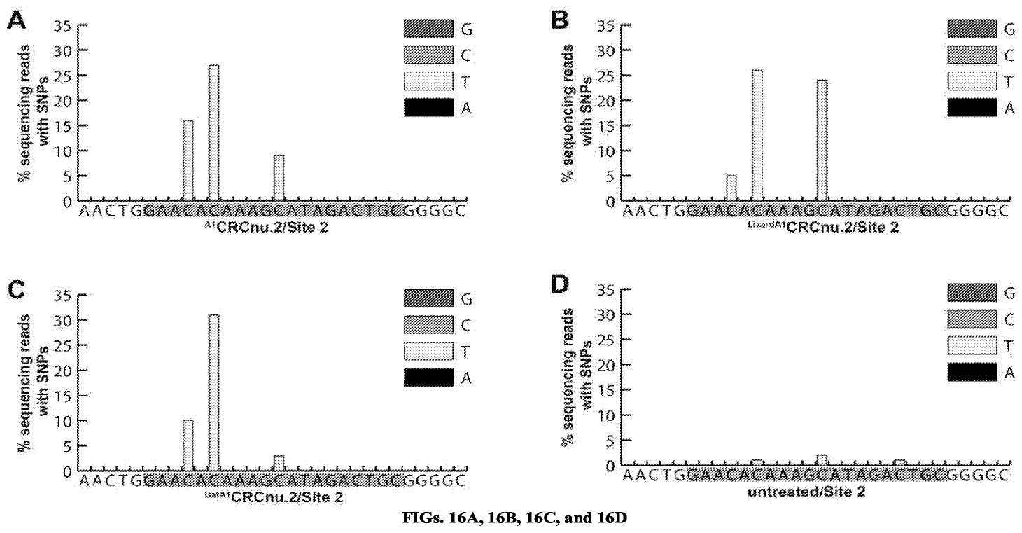

As disclosed herein, this invention provides further engineering of a second-generation CRC system for precision base editing. As demonstrated herein, the second-generation CRC system exhibits a number of important different features compared to the previous CRC system (first generation) described in WO2018129129 and WO2017011721. The second generation CRC system exhibit substantially increased on-target efficacy compared to the first generation CRC. Among the Second Generation CRCs, we optimized the configurations selecting the ones with higher efficacy, lower or lack of off-target effect, higher purity (more C to T conversion rather than C to other nucleotides). Importantly, when second generation CRC system utilizes a wide variety of cytidine deaminases from different species and different deaminase families were tested, many of them show clear different activity windows and preference positions from any previously described base editing systems including BE systems, as well as higher activity. See, e.g., FIGS. 16-20.

a. Sequence-Targeting Module

The sequence-targeting component of the above system is based on CRISPR/Cas systems from bacterial species. The original functional bacterial CRISPR-Cas system requires three components: the Cas protein, which provides the nuclease activity and two short, non-coding RNA species, referred to as CRISPR RNA (crRNAs) and trans-acting RNA (tracrRNA), which two RNA species form a so-called guide RNA (gRNA). Type II CRISPR is one of the most well characterized systems and carries out targeted DNA double-strand break in four sequential steps. First, two non-coding RNAs, a pre-crRNA and a tracrRNA, are transcribed from a CRISPR locus. Second, the tracrRNA hybridizes to the repeat regions of the pre-crRNA molecules and mediates processing of pre-crRNA molecules into mature crRNA molecules containing individual spacer sequences. Third, a mature crRNA:tracrRNA complex (i.e., the so-called guide RNA) directs a Cas nuclease (such as Cas9) to target DNA via Watson-Crick base-pairing between the spacer sequence on the crRNA and the complement of the protospacer sequence on the target DNA, which comprises a 3-nucleotide (nt) protospacer adjacent motif (PAM). PAM sequences are essential for Cas9 targeting. Finally, the Cas nuclease mediates cleavage of the target DNA to create a double-stranded break within the target site. In its native context, a CRISPR/Cas system acts as an adaptive immune system that protects bacteria from repeated viral infections, and PAM sequences serve as self/non-self-recognition signals, and Cas9 protein has nuclease activity. CRISPR/Cas systems have been shown to have enormous potential for gene editing, both in vitro and in vivo.

In the invention disclosed herein, the sequence recognition mechanism can be achieved in a similar manner That is, a mutant Cas protein, for example, a dCas9 protein which contains mutations at its nuclease catalytic domains thus does not have nuclease activity, or a nCas9 protein which is partially mutated at one of the catalytic domains thus does not have nuclease activity for generating DSB, specifically recognizes a non-coding RNA scaffold molecule containing a short spacer sequence, typically 20 nucleotides in length, which guides the Cas protein to its target DNA or RNA sequence. The latter is flanked by a 3′ PAM.

Cas Proteins

Various Cas proteins can be used in this invention. A Cas protein, CRISPR-associated protein, or CRISPR protein, used interchangeably, refers to a protein of or derived from a CRISPR-Cas type I, type II, or type III system, which has an RNA-guided DNA-binding. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. See e.g., WO2014144761, WO2014144592, WO2013176772, US20140273226, and US20140273233, the contents of which are incorporated herein by reference in their entireties.

In one embodiment, the Cas protein is derived from a type II CRISPR-Cas system. In exemplary embodiments, the Cas protein is or is derived from a Cas9 protein. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum the rmopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

In general, a Cas protein includes at least one RNA binding domain. The RNA binding domain interacts with the guide RNA. The Cas protein can be a wild type Cas protein or a modified version with no nuclease activity. The Cas protein can be modified to increase nucleic acid binding affinity and/or specificity, alter an enzymatic activity, and/or change another property of the protein. For example, nuclease (i.e., DNase, RNase) domains of the protein can be modified, deleted, or inactivated. Alternatively, the protein can be truncated to remove domains that are not essential for the function of the protein. The protein can also be truncated or modified to optimize the activity of the effector domain.

In some embodiments, the Cas protein can be a mutant of a wild type Cas protein (such as Cas9) or a fragment thereof. In other embodiments, the Cas protein can be derived from a mutant Cas protein. For example, the amino acid sequence of the Cas9 protein can be modified to alter one or more properties (e.g., nuclease activity, affinity, stability, etc.) of the protein. Alternatively, domains of the Cas9 protein not involved in RNA targeting can be eliminated from the protein such that the modified Cas9 protein is smaller than the wild type Cas9 protein. In some embodiments, the present system utilizes the Cas9 protein from S. pyogenes, either as encoded in bacteria or codon-optimized for expression in mammalian cells.

A mutant Cas protein refers to a polypeptide derivative of the wild type protein, e.g., a protein having one or more point mutations, insertions, deletions, truncations, a fusion protein, or a combination thereof. The mutant has at least one of the RNA-guided DNA binding activity, or RNA-guided nuclease activity, or both. In general, the modified version is at least 50% (e.g., any number between 50% and 100%, inclusive, e.g., 50%, 60%, 70%, 75%, 80%, 85%, 90%, 95%, and 99%) identical to the wild type protein such as SEQ ID NO: 1 (from GenBank: AKE81011.1) below:

DKKYSIGLDIGTNSVGWAVITDEYKVPSKKEKVLGNTDRHSIKKNLIGA
LLEDSGETAEATRLKRTARRRYTRRKNRICYLQEIFSNEMAKVDDSFEH
RLEESELVEEDKKHERHPIFGNIVDEVAYHEKYPTIYHLRKKLVDSTDK
ADLRLIYLALAHMIKERGHFLIEGDLNPDNSDVDKLFIQLVQTYNQLFE
ENPINASGVDAKAILSARLSKSRRLENLIAQLPGEKKNGLFGNLIALSL
GLIPNEKSNEDLAEDAKLQLSKDTYDDDLDNLLAQIGDQYADLFLAAKN
LSDAILLSDILRVNTEITKAPLSASMIKRYDEHHQDLTLLKALVRQQLP
EKYKEIFFDQSKNGYAGYIDGGASQEEFYKFIKPILEKMDGTEELLVKL
NREDLLRKQRTFDNGSIPHQIHLGELHAILRRQEDFYPFLKDNREKIEK
ILTFRIPYYVGPLARGNSRFAWMTRKSEETITPWNFEEVVDKGASAQSF
IERMTNEDKNLPNEKVLPKHSLLYEYFTVYNELTKVKYVTEGMRKPAFL
SGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDG
FANRNFMQLIHDDSLTEKEDIQKAQVSGQGDSLHEHIANLAGSPAIKKG
ILQTVKVVDELVKVMGRHKPENIVIEMARENQTTQKGQKNSRERMKRIE
EGIKELGSQILKEHPVENTQLQNEKLYLYYLQNGRDMYVDQELDINRLS
DYDVDHIVPQSFLKDDSIDNKVLTRSDKNRGKSDNVPSEEVVKKMKNYW
RQLLNAKLITQRKFDNLTKAERGGLSELDKAGFIKRQLVETRQITKHVA
QILDSRMNTKYDENDKLIREVKVITLKSKLVSDFRKDFQFYKVREINNY
HHAHDAYLNAVVGTALIKKYPKLESEFVYGDYKVYDVRKMIAKSEQEIG
KATAKYFFYSNIMNFEKTEITLANGEIRKRPLIEINGETGEIVWDKGRD
FATVRKVLSMPQVNIVKKTEVQTGGFSKESILPKRNSDKLIARKKDWDP
KKYGGFDSPTVAYSVLVVAKVEKGKSKKLKSVKELLGITIMERSSFEKN
PIDFLEAKGYKEVKKDLIIKLPKYSLFELENGRKRMLASAGELQKGNEL
ALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAF
KYFDTTIDRKRYTSTKEVLDATLIHQSITGLYETRIDLSQLGGD

A Cas protein (as well as other protein components described in this invention) can be obtained as a recombinant polypeptide. To prepare a recombinant polypeptide, a nucleic acid encoding it can be linked to another nucleic acid encoding a fusion partner, e.g., glutathione-s-transferase (GST), 6×-His epitope tag, or M13 Gene 3 protein. The resultant fusion nucleic acid expresses in suitable host cells a fusion protein that can be isolated by methods known in the art. The isolated fusion protein can be further treated, e.g., by enzymatic digestion, to remove the fusion partner and obtain the recombinant polypeptide of this invention. Alternatively, the proteins can be chemically synthesized (see e.g., Creighton, “Proteins: Structures and Molecular Principles,” W.H. Freeman & Co., NY, 1983), or produced by recombinant DNA technology as described herein. For additional guidance, skilled artisans may consult Frederick M. Ausubel et al., Current Protocols in Molecular Biology, John Wiley & Sons, 2003; and Sambrook et al., Molecular Cloning, A Laboratory Manual,” Cold Spring Harbor Press, Cold Spring Harbor, N.Y., 2001).

The Cas protein described in the invention can be provided in purified or isolated form, or can be part of a composition. Preferably, where in a composition, the proteins are first purified to some extent, more preferably to a high level of purity (e.g., about 80%, 90%, 95%, or 99% or higher). Compositions according to the invention can be any type of composition desired, but typically are aqueous compositions suitable for use as, or inclusion in, a composition for RNA-guided targeting. Those of skill in the art are well aware of the various substances that can be included in such nuclease reaction compositions.

To practice the method disclosed herein for modifying a target nucleic acid, one can produce the proteins in a target cell via mRNA, protein RNA complexes (RNP), or any suitable expression vectors. Examples of expression vectors include chromosomal, non-chromosomal and synthetic DNA sequences, bacterial plasmids, minicircles, phage DNA, baculovirus, yeast plasmids, vectors derived from combinations of plasmids and phage DNA, viral DNA such as vaccinia, adenovirus, fowl pox virus, and pseudorabies. More details are described in the Expression System and Methods sections below.

As disclosed here, one can use the nuclease dead Cas9 (dCas9, for example from S. pyogenes D10A, _{H840A} mutant protein), or the nuclease defective nickase Cas9 (nCas9, for example from S. pyogenes D10A mutant protein). dCas9 or nCas9 could also be derived from various bacterial species. Table 1 lists a non-exhausting list of examples of dCas9, and their corresponding PAM requirements. One can also use synthetic Cas substitutes such as those described in Rauch et al., Programmable RNA-Guided RNA Effector Proteins Built from Human Parts. Cell Volume 178, Issue 1, 27 Jun. 2019, Pages 122-134.e12.

TABLE 1

| Species | PAM |
|---|---|
| Streptococcus pyogenes | NGG |
| Streptococcus agalactiae | NGG |
| Staphylococcus aureus | NNGRRT |
| Streptococcus thermophilus | NNAGAAW |
| Streptococcus thermophilus | NGGNG |
| Neisseria meningitidis | NNNNGATT |
| Treponema denticola | NAAAAC |
| Other Type II CRISPR/Cas9 | |
| systems from other bacterial | |
| species | |

UGI

In some aspects of this disclosure, the above-described sequence-targeting component comprises a target fusion protein having (a) a sequence-targeting protein, and (b) a first uracil DNA glycosylase (UNG) inhibitor peptide (UGI). For example, the fusion protein can include a Cas9 protein fused to a UGI. Such fusion proteins may exhibit an increased nucleic acid editing efficiency as compared to fusion proteins not comprising an UGI domain. In some embodiments, the UGI comprises a wild type UGI sequence or one having the following amino acid sequence: spIP14739IUNGI_BPPB2: Uracil-DNA glycosylase inhibitor (UGI) MTNLSDIIEKETGKQLVIQESILMLPEEVEEVIGNKPESDILVHTAYDESTDENVMLLTS DAPEYKPWALVIQDSNGENKIKML (SEQ ID NO: 44).

In some embodiments, the UGI proteins provided herein include fragments of UGI and proteins homologous to a UGI or a UGI fragment. For example, in some embodiments, a UGI comprises a fragment of the amino acid sequence set forth above. In some embodiments, a UGI comprises an amino acid sequence homologous to the amino acid sequence set forth above or an amino acid sequence homologous to a fragment of the amino acid sequence set forth in the UGI sequence above. In some embodiments, proteins comprising UGI or fragments of UGI or homologs of UGI or UGI fragments are referred to as “UGI variants.” A UGI variant shares homology to UGI, or a fragment thereof. For example, a UGI variant is at least about 70% (e.g., at least about 80%, 90%, 95%, 96%, 97%, 98%, 99%) to a wild type UGI or the UGI sequence as set forth above.

Suitable UGI protein and nucleotide sequences are provided herein and additional suitable UGI sequences are known to those in the art, and include, for example, those published in Wang et al., Uracil-DNA glycosylase inhibitor gene of bacteriophage PBS2 encodes a binding protein specific for uracil-DNA glycosylase. J Biol. Chem. 264:1163-1171(1989); Lundquist et al., Site-directed mutagenesis and characterization of uracil-DNA glycosylase inhibitor protein. Role of specific carboxylic amino acids in complex formation with Escherichia coli uracil-DNA glycosylase. J Biol. Chem. 272:21408-21419(1997); Ravishankar et al., X-ray analysis of a complex of Escherichia coli uracil DNA glycosylase (EcUDG) with a proteinaceous inhibitor. The structure elucidation of a prokaryotic UDG. Nucleic Acids Res. 26:4880-4887(1998); and Putnam et al., Protein mimicry of DNA from crystal structures of the uracil-DNA glycosylase inhibitor protein and its complex with Escherichia coli uracil-DNA glycosylase. J Mol. Biol. 287:331-346(1999), the entire contents of each are incorporated herein by reference.

b. RNA Scaffold for Sequence Recognition and Effector Recruitment:

The second component of the platform disclosed herein is an RNA scaffold, which has three sub-components: a programmable guide RNA motif, a CRISPR RNA motif, and a recruiting RNA motif. This scaffold can be either a single RNA molecule or a complex of multiple RNA molecules. As disclosed herein, the programmable guide RNA, CRISPR RNA and the Cas protein together form a CRISPR/Cas-based module for sequence targeting and recognition, while the recruiting RNA motif via an RNA-protein binding pair recruits a protein effector, which carries out genetic correction. Accordingly, this second component connects the correction module and sequence recognition module.

Programmable Guide RNA

One key sub-component is the programmable guide RNA. Due to its simplicity and efficiency, the CRISPR-Cas system has been used to perform genome-editing in cells of various organisms. The specificity of this system is dictated by base pairing between a target DNA and a custom-designed guide RNA. By engineering and adjusting the base-pairing properties of guide RNAs, one can target any sequences of interest provided that there is a PAM sequence in a target sequence.

Among the sub-components of the RNA scaffold disclosed herein, the guide sequence provides the targeting specificity. It includes a region that is complementary and capable of hybridization to a pre-selected target site of interest. In various embodiments, this guide sequence can comprise from about 10 nucleotides to more than about 25 nucleotides. For example, the region of base pairing between the guide sequence and the corresponding target site sequence can be about 10, 11, 12, 13, 14, 15, 16, 17, 18, 19, 20, 22, 23, 24, 25, or more than 25 nucleotides in length. In an exemplary embodiment, the guide sequence is about 17-20 nucleotides in length, such as 20 nucleotides.

One requirement for selecting a suitable target nucleic acid is that it has a 3′ PAM site/sequence. Each target sequence and its corresponding PAM site/sequence are referred herein as a Cas-targeted site. The Type II CRISPR system, one of the most well characterized systems, needs only Cas9 protein and a guide RNA complementary to a target sequence to affect target cleavage. The type II CRISPR system of S. pyogenes uses target sites having N12-20NGG, where NGG represents the PAM site from S. pyogenes, and N12-20 represents the 12-20 nucleotides directly 5′ to the PAM site. Additional PAM site sequences from other species of bacteria include NGGNG, NNNNGATT, NNAGAA, NNAGAAW, and NAAAAC. See, e.g., US 20140273233, WO 2013176772, Cong et al., (2012), Science 339 (6121): 819-823, Jinek et al., (2012), Science 337 (6096): 816-821, Mali et al, (2013), Science 339 (6121): 823-826, Gasiunas et al., (2012), Proc Natl Acad Sci USA. 109 (39): E2579-E2586, Cho et al., (2013) Nature Biotechnology 31, 230-232, Hou et al., Proc Natl Acad Sci USA. 2013 Sep. 24; 110(39):15644-9, Mojica et al., Microbiology. 2009 March; 155(Pt 3):733-40, and www.addgene.org/CRISPR/. The contents of these documents are incorporated herein by reference in their entireties.

The target nucleic acid strand can be either of the two strands on a genomic DNA in a host cell. Examples of such genomic dsDNA include, but are not necessarily limited to, a host cell chromosome, mitochondrial DNA and a stably maintained plasmid. However, it is to be understood that the present method can be practiced on other dsDNA present in a host cell, such as non-stable plasmid DNA, viral DNA, and phagemid DNA, as long as there is Cas-targeted site regardless of the nature of the host cell dsDNA. The present method can be practiced on RNAs too.

CRISPR Motif

Besides the above-described guide sequence, the RNA scaffold of this invention includes additional active or non-active sub-components. In one example, the scaffold has a CRISPR motif with tracrRNA activity. For example, the scaffold can be a hybrid RNA molecule where the above-described programmable guide RNA is fused to a tracrRNA to mimic the natural crRNA:tracrRNA duplex. Shown below is an exemplary hybrid crRNA:tracrRNA, gRNA sequence: 5′-(20nt guide)-GUUUAAGAGCUAUGCUGGAAACAGCAUAGCAAGUUUAAAUAAGGC UAGUCCGUUAUCAACUUGAAAAAGUGGCACCGAGUCGGUGCUUUUUUU-3′ (SEQ ID NO: 45; Chen et al. Cell. 2013 Dec. 19; 155(7):1479-91). Various tracrRNA sequences are known in the art and examples include the following tracrRNAs and active portions thereof. As used herein, an active portion of a tracrRNA retains the ability to form a complex with a Cas protein, such as Cas9 or dCas9. See, e.g., WO2014144592. Methods for generating crRNA-tracrRNA hybrid RNAs are known in the art. See e.g., WO2014099750, US 20140179006, and US 20140273226. The contents of these documents are incorporated herein by reference in their entireties.

(SEQ ID NO: 46)
GGAACCAUUCAAAACAGCAUAGCAAGUUAAAAUAAGGCUAGUCCGUUAU
CAACUUGAAAAAGUGGCACCGAGUCGGUGC;
(SEQ ID NO: 47)
UAGCAAGUUAAAAUAAGGCUAGUCCGUUAUCAACUUGAAAAAGUGGCAC
CGAGUCGGUGC;
(SEQ ID NO: 48)
AGCAUAGCAAGUUAAAAUAAGGCUAGUCCGUUAUCAACUUGAAAAAGUG
GCACCGAGUCGGUGC;
(SEQ ID No: 49)
CAAAACAGCAUAGCAAGUUAAAAUAAGGCUAGUCCGUUAUCAACUUGAA
AAAGUGGCACCGAGUCGGUGC;
(SEQ ID NO: 50)
UAGCAAGUUAAAAUAAGGCUAGUCCGUUAUCAACUUGAAAAAGUG;
(SEQ ID NO: 51)
UAGCAAGUUAAAAUAAGGCUAGUCCGUUAUCA;
and
(SEQ ID NO: 52)
UAGCAAGUUAAAAUAAGGCUAGUCCG.

In some embodiments, the tracrRNA activity and the guide sequence are two separate RNA molecules, which together form the guide RNA and related scaffold. In this case, the molecule with the tracrRNA activity should be able to interact with (usually by base pairing) the molecule having the guide sequence.

Recruiting RNA Motif

The third sub-component of the RNA scaffold is the recruiting RNA motif(s), which links the correction module and sequence recognition module. This linkage is critical for the platform disclosed herein.

One way to recruit effector/DNA editing enzymes to a target sequence is through a direct fusion of an effector protein to dCas9. The direct fusion of effector enzymes (“correction module”) to the proteins required for sequence recognition (such as dCas9) has achieved success in sequence specific transcriptional activation or suppression, but the protein-protein fusion design may render spatial hindrance, which is not ideal for enzymes that need to form a multimeric complex for their activities. In fact, most nucleotide editing enzymes (such as AID or APOBEC3G) require formation of dimers, tetramers or higher order oligomers, for their DNA editing catalytic activities.

In contrast, the platform disclosed herein is based on RNA scaffold-mediated effector protein recruitment. More specifically, the platform takes advantage of various RNA motif/RNA binding protein binding pairs. To this end, an RNA scaffold is designed such that an RNA motif (e.g., MS2 operator motif), which specifically binds to an RNA binding protein (e.g., MS2 coat protein, MCP), is linked to the gRNA-CRISPR scaffold. The recruiting RNA motif can be fused to the 3′ or 5′ ends of the gRNA-CRISPR scaffold, or it could replace the loops within the gRNA-CRISPR scaffold, specifically the tetraloop and/or stem loop 2.

As a result, this RNA scaffold component of the platform disclosed herein is a designed RNA molecule, which contains not only the gRNA motif for specific DNA/RNA sequence recognition, the CRISPR RNA motif for dCas9 binding, but also the recruiting RNA motif for effector recruitment (FIG. 1B). In this way, recruited-effector protein fusions can be recruited to the target site through their ability to bind to the recruiting RNA motif. Due to the flexibility of RNA scaffold mediated recruitment, a functional monomer, as well as dimer, tetramer, or oligomer could be relatively easy to form near the target DNA or RNA sequence. These pairs of RNA recruiting motif/binding protein could be derived from naturally occurring sources (e.g., RNA phages, or yeast telomerase) or could be artificially designed (e.g., RNA aptamers and their corresponding binding protein ligands). A non-exhaustive list of examples of recruiting RNA motif/RNA binding protein pairs that could be used in the CasRcure system is summarized in Table 2.

TABLE 2
Examples of recruiting RNA motifs that can be used in this invention,
as well as their paring RNA binding proteins/protein domains.

| | Pairing interacting | |
|---|---|---|
| RNA motif | protein* | Organism |
| Telomerase Ku binding motif | Ku | Yeast |
| Telomerase Sm7 binding motif | Sm7 | Yeast |
| MS2 phage operator stem-loop | MS2 Coat Protein | Phage |
| | (MCP) | |
| PP7 phage operator stem-loop | PP7 coat protein | Phage |
| | (PCP) | |
| SfMu phage Com stem-loop | Com RNA binding | Phage |
| | protein | |
| Non-natural RNA aptamer | Corresponding | Artificially |
| | aptamer ligand | designed |

*Recruited proteins are fused to effector proteins, for examples see Table 3.

The sequences for the above binding pairs are listed below.

1. Telomerase Ku biding motif/Ku heterodimer
a. Ku binding hairpin
5′-
UUCUUGUCGUACUUAUAGAUCGCUACGUUAUUUCAAUUUUGAAAAUCUGAGUCCUGGGAGUGC
GGA-3′ (SEQ ID No: 53)
b. Ku heterodimer
MSGWESYYKTEGDEEAEEEQEENLEASGDYKYSGRDSLIFLVDASKAMFESQSEDELTPFDMS
IQCIQSVYISKIISSDRDLLAVVFYGTEKDKNSVNFKNIYVLQELDNPGAKRILELDQFKGQQ
GQKRFQDMMGHGSDYSLSEVLWVCANLFSDVQFKMSHKRIMLFTNEDNPHGNDSAKASRARTK
AGDLRDTGIFLDLMHLKKPGGFDISLFYRDIISIAEDEDLRVHFEESSKLEDLLRKVRAKETR
KRALSRLKLKLNKDIVISVGIYNLVQKALKPPPIKLYRETNEPVKTKTRTFNTSTGGLLLPSD
TKRSQIYGSRQIILEKEETEELKRFDDPGLMLMGFKPLVLLKKHHYLRPSLFVYPEESLVIGS
STLFSALLIKCLEKEVAALCRYTPRRNIPPYFVALVPQEEELDDQKIQVTPPGFQLVFLPFAD
DKRKMPFTEKIMATPEQVGKMKAIVEKLRFTYRSDSFENPVLQQHFRNLEALALDLMEPEQAV
DLTLPKVEAMNKRLGSLVDEFKELVYPPDYNPEGKVTKRKHDNEGSGSKRPKVEYSEEELKTH
ISKGTLGKFTVPMLKEACRAYGLKSGLKKQELLEALTKHFQD (SEQ ID No: 54)
MVRSGNKAAVVLCMDVGFTMSNSIPGIESPFEQAKKVITMFVQRQVFAENKDEIALVLFGTDG
TDNPLSGGDQYQNITVHRHLMLPDFDLLEDIESKIQPGSQQADFLDALIVSMDVIQHETIGKK
FEKRHIEIFTDLSSRFSKSQLDIIIHSLKKCDISERHSIHWPCRLTIGSNLSIRIAAYKSILQ
ERVKKTWTVVDAKTLKKEDIQKETVYCLNDDDETEVLKEDIIQGFRYGSDIVPFSKVDEEQMK
YKSEGKCFSVLGFCKSSQVQRRFFMGNQVLKVFAARDDEAAAVALSSLIHALDDLDMVAIVRY
AYDKRANPQVGVAFPHIKHNYECLVYVQLPFMEDLRQYMFSSLKNSKKYAPTEAQLNAVDALI
DSMSLAKKDEKTDTLEDLFPTTKIPNPRFQRLFQCLLHRALHPREPLPPIQQHIWNMLNPPAE
VTTKSQIPLSKIKTLFPLIEAKKKDQVTAQEIFQDNHEDGPTAK (SEQ ID No: 55)
2. Telomerase Sm7 biding motif/Sm7 homoheptamer
a. Sm consensus site (single stranded)
5′-AAUUUUUGGA-3′ (SEQ ID NO: 56)
b. Monomeric Sm-like protein (archaea)
GSVIDVSSQRVNVQRPLDALGNSLNSPVIIKLKGDREFRGVLKSFDLHMNLVLNDAEELEDGE
VTRRLGTVLIRGDNIVYISP (SEQ ID NO: 57)
3. MS2 phage operator stem loop/MS2 coat protein
a. MS2 phage operator stem loop
5′-GCGCACAUGAGGAUCACCCAUGUGC-3′ (SEQ ID NO: 58)
b. MS2 coat protein
MASNFTQFVLVDNGGTGDVTVAPSNFANGIAEWISSNSRSQAYKVTCSVRQSSAQNRKYTIKV
EVPKGAWRSYLNMELTIPIFATNSDCELIVKAMQGLLKDGNPIPSAIAANSGIY (SEQ ID
NO: 59)
4. PP7 phage operator stem loop/PP7 coat protein
a. PP7 phage operator stem loop
5′-AUAAGGAGUUUAUAUGGAAACCCUUA-3′ (SEQ ID NO: 60)
b. PP7 coat protein (PCP)
MSKTIVLSVGEATRTLTEIQSTADRQIFEEKVGPLVGRLRLTASLRQNGAKTAYRVNLKLDQA
DVVDCSTSVCGELPKVRYTQVWSHDVTIVANSTEASRKSLYDLTKSLVATSQVEDLVVNLVPL
GR (SEQ ID NO: 61)
5. SfMu Com stem loop/SfMu Com binding protein
a. SfMu Com stem loop
5′-CUGAAUGCCUGCGAGCAUC-3′ (SEQ ID NO: 62)
b. SfMu Com binding protein
MKSIRCKNCNKLLFKADSFDHIEIRCPRCKRHIIMLNACEHPTEKHCGKREKITHSDETVRY
(SEQ ID NO: 63)

The RNA scaffold can be either a single RNA molecule or a complex of multiple RNA molecules. For example, the guide RNA, CRISPR motif, and recruiting RNA motif can be three segments of one, long single RNA molecule. Alternatively, one, two or three of them can be on separate molecules. In the latter case, the three components can be linked together to form the scaffold via covalent or non-covalent linkage or binding, including e.g., Watson-Crick base-pairing.

In one example, the RNA scaffold can comprise two separate RNA molecules. The first RNA molecule can comprise the programmable guide RNA and a region that can form a stem duplex structure with a complementary region. The second RNA molecule can comprise the complementary region in addition to the CRISPR motif and the recruiting DNA motif. Via this stem duplex structure, the first and second RNA molecules form an RNA scaffold of this invention. In one embodiment, the first and second RNA molecules each comprise a sequence (of about 6 to about 20 nucleotides) that base pairs to the other sequence. By the same token, the CRISPR motif and the recruiting DNA motif can also be on different RNA molecule and be brought together with another stem duplex structure.

The RNAs and related scaffold of this invention can be made by various methods known in the art including cell-based expression, in vitro transcription, and chemical synthesis. The ability to chemically synthesize relatively long RNAs (as long as 200 mers or more) using TC-RNA chemistry (see, e.g., U.S. Pat. No. 8,202,983) allows one to produce RNAs with special features that outperform those enabled by the basic four ribonucleotides (A, C, G and U).

The Cas protein-guide RNA scaffold complexes can be made with recombinant technology using a host cell system or an in vitro translation-transcription system known in the art. Details of such systems and technology can be found in e.g., WO2014144761 WO2014144592, WO2013176772, US20140273226, and US20140273233, the contents of which are incorporated herein by reference in their entireties. The complexes can be isolated or purified, at least to some extent, from cellular material of a cell or an in vitro translation-transcription system in which they are produced.

Modifications

The RNA scaffold may include one or more modifications. Such modifications may include inclusion of at least one non-naturally occurring nucleotide, or a modified nucleotide, or analogs thereof. Modified nucleotides may be modified at the ribose, phosphate, and/or base moiety. Modified nucleotides may include 2′-O-methyl analogs, 2′-deoxy analogs, or 2′-fluoro analogs. The nucleic acid backbone may be modified, for example, a phosphorothioate backbone may be used. The use of locked nucleic acids (LNA) or bridged nucleic acids (BNA) may also be possible. Further examples of modified bases include, but are not limited to, 2-aminopurine, 5-bromo-uridine, pseudouridine, inosine, 7-methylguanosine. These modifications may apply to any component of the CRISPR system. In a preferred embodiment these modifications are made to the RNA components, e.g., the guide RNA sequence.

In some embodiments, the RNA scaffold described above or a subsection thereof can comprise one or more modifications, e.g., a base modification, a backbone modification, etc, to provide the nucleic acid with a new or enhanced feature (e.g., improved stability).

Modified Backbones and Modified Inter-Nucleoside Linkages

Examples of suitable nucleic acids containing modifications include nucleic acids containing modified backbones or non-natural internucleoside linkages. Nucleic acids (having modified backbones include those that retain a phosphorus atom in the backbone and those that do not have a phosphorus atom in the backbone.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

In some embodiments, a subject nucleic acid comprises one or more phosphorothioate and/or heteroatom internucleoside linkages, in particular —CH₂—NH—O—CH₂—, —CH₂—N(CH₃)—O—CH₂-(known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). MMI type internucleoside linkages are disclosed in the above referenced U.S. Pat. No. 5,489,677. Suitable amide internucleoside linkages are disclosed in t U.S. Pat. No. 5,602,240.

Also suitable are nucleic acids having morpholino backbone structures as described in, e.g., U.S. Pat. No. 5,034,506. For example, in some embodiments, a subject nucleic acid comprises a 6-membered morpholino ring in place of a ribose ring. In some of these embodiments, a phosphorodiamidate or other non-phosphodiester internucleoside linkage replaces a phosphodiester linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Mimetics

A subject nucleic acid can be a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has been reported to have excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Dwaine A. Braasch and David R. Corey, Biochemistry, 2002, 41(14), 4503-4510). Morpholino-based polynucleotides are disclosed in U.S. Pat. No. 5,034,506. A variety of compounds within the morpholino class of polynucleotides have been prepared, having a variety of different linking groups joining the monomeric subunits.

A further class of polynucleotide mimetic is referred to as cyclohexenyl nucleic acids (CeNA). The furanose ring normally present in a DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation.

A further modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH2-), group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tm=+3 to +10° C.), stability towards 3′-exonucleolytic degradation and good solubility properties. Potent and nontoxic antisense oligonucleotides containing LNAs have been described (Wahlestedt et al., Proc. Natl. Acad. Sci. U.S.A., 2000, 97, 5633-5638).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

Modified Sugar Moieties

A subject nucleic acid can also include one or more substituted sugar moieties. Suitable polynucleotides comprise a sugar substituent group selected from: OH; F; O-, S-, or N-alkyl; O-, S-, or N-alkenyl; O-, S- or N-alkynyl; or O-alkyl-Co-alkyl, wherein the alkyl, alkenyl and alkynyl may be substituted or unsubstituted C₁ to C₁₀ alkyl or C₂ to C₁₀ alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′—O—CH₂CH₂OCH₃, also known as 2′-O-(2-methoxyethyl) or 2′-MOE) (Martin et al., Helv. Chim Acta, 1995, 78, 486-504) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′—O—CH₂—O—CH₂—N(CH₃)₂.

Other suitable sugar substituent groups include methoxy (—O—CH₃), aminopropoxy (—OCH₂CH₂NH₂), allyl (—CH₂—CH═CH₂), —O-allyl CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

Base Modifications and Substitutions

A subject nucleic acid may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido(5,4-b) (1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are useful for increasing the binding affinity of an oligomeric compound. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278) and are suitable base substitutions, e.g., when combined with 2′-O-methoxyethyl sugar modifications.

c. Effectors: Non-Nuclease DNA Modifying Enzymes

The third component of the platform disclosed in this invention is a non-nuclease effector. The effector is not a nuclease and does not have any nuclease activity but can have the activity of other types of DNA modifying enzymes. Examples of the enzymatic activity include, but are not limited to, deamination activity, methyltransferase activity, demethylase activity, DNA repair activity, DNA damage activity, dismutase activity, nickase activity, alkylation activity, depurination or depyrimidination activity, oxidation activity, pyrimidine dimer forming activity, integrase activity, transposase activity, recombinase activity, polymerase activity, ligase activity, helicase activity, photolyase activity or glycosylase activity. In some embodiments, the effector has the activity of cytidine deaminases (e.g., AID, APOBEC3G, and APOBEC1), adenosine deaminases (e.g., ADA), DNA methyltransferases, and DNA demethylases. In some embodiments, the effectors are from different vertebrate animal species have distinct activity properties.

In preferred embodiments, this third component is a conjugate or a fusion protein that has an RNA-binding domain and an effector domain. These two domains can be joined via a linker.

In some embodiments, no effector is needed in some cell types (e.g., cancer lines over-expressing demainases). In that case, endogenous effector (e.g. APOBEC, AID, etc) can be gene-edited to include the recruitment module, so no exogenous editor is needed. This is applicable to cell types that express the editor of interest—e.g., lymphoid (B+T cells) and certain cancer cells. In addition, the nickase activity does not have to come from the Cas module but can be recruited from the effectors—for example, dCas9 can have an aptamer to recruit both the nickase and editor via the same gRNA recruitment.

RNA-Binding Domain

Although various RNA-binding domains can be used in this invention, the RNA-binding domain of Cas protein (such as Cas9) or its variant (such as dCas9) should not be used. As mentioned above, the direct fusion to dCas9, which anchors to DNA in a defined conformation, would hinder the formation of a functional oligomeric enzyme complex at the right location. Instead, the present invention takes advantages of various other RNA motif-RNA binding protein binding pairs. Examples include those listed in Table 2.

In this way, the effector protein can be recruited to the target site through RNA-binding domain's ability to bind to the recruiting RNA motif. Due to the flexibility of RNA scaffold mediated recruitment, a functional monomer, as well as dimer, tetramer, or oligomer could be formed relatively easily near the target DNA or RNA sequence.

Effector Domain

The effector component comprises an activity portion, i.e., an effector domain. In some embodiments, the effector domain comprises the naturally occurring activity portion of a non-nuclease protein (e.g., deaminases). In other embodiments, the effector domain comprises a modified amino acid sequence (e.g., substitution, deletion, insertion) of a naturally occurring activity portion of a non-nuclease protein. The effector domain has an enzymatic activity. Examples of this activity include deamination activity, methyltransferase activity, demethylase activity, DNA repair activity, DNA damage activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, DNA methylation, histone acetylation activity, or histone methylation activity. Some modifications in non-nuclease protein (e.g., deaminases) can help reduce off-target effect. For example, as described below, one can reduce the recruitment of AID to off-target sites by mutating Ser38 in AID to Ala.

Linker

The above-mentioned two domains as well as others as disclosed herein can be joined by means of linkers, such as, but not limited to chemical modification, peptide linkers, chemical linkers, covalent or non-covalent bonds, or protein fusion or by any means known to one skilled in the art. The joining can be permanent or reversible. See for example U.S. Pat. Nos. 4,625,014, 5,057,301 and 5,514,363, US Application Nos. 20150182596 and 20100063258, and WO2012142515, the contents of which are incorporated herein in their entirety by reference. In some embodiments, several linkers can be included in order to take advantage of desired properties of each linker and each protein domain in the conjugate. For example, flexible linkers and linkers that increase the solubility of the conjugates are contemplated for use alone or with other linkers. Peptide linkers can be linked by expressing DNA encoding the linker to one or more protein domains in the conjugate. Linkers can be acid cleavable, photocleavable and heat sensitive linkers. Methods for conjugation are well known by persons skilled in the art and are encompassed for use in the present invention.

In some embodiments, the RNA-binding domain and the effector domain can be joined by a peptide linker. Peptide linkers can be linked by expressing nucleic acid encoding in frame the two domains and the linker. Optionally the linker peptide can be joined at either or both of the amino terminus and carboxy terminus of the domains. In some examples, a linker is an immunoglobulin hinge region linker as disclosed in U.S. Pat. Nos. 6,165,476, 5,856,456, US Application Nos. 20150182596 and 2010/0063258 and International Application WO2012/142515, each of which are incorporated herein in their entirety by reference.

Other Domains

The effector fusion protein can comprise other domains. In certain embodiments, the effector fusion protein can comprise at least one nuclear localization signal (NLS). In general, an NLS comprises a stretch of basic amino acids. Nuclear localization signals are known in the art (see, e.g., Lange et al., J. Biol. Chem., 2007, 282:5101-5105). The NLS can be located at the N-terminus, the C-terminal, or in an internal location of the fusion protein.

In some embodiments, the fusion protein can comprise at least one cell-penetrating domain to facilitate delivery of the protein into a target cell. In one embodiment, the cell-penetrating domain can be a cell-penetrating peptide sequence. Various cell-penetrating peptide sequences are known in the art and examples include that of the HIV-1 TAT protein, TLM of the human HBV, Pep-1, VP22, and a polyarginine peptide sequence.

In still other embodiments, the fusion protein can comprise at least one marker domain. Non-limiting examples of marker domains include fluorescent proteins, purification tags, and epitope tags. In some embodiments, the marker domain can be a fluorescent protein. In other embodiments, the marker domain can be a purification tag and/or an epitope tag. See, e.g., US 20140273233.

In one embodiment, AID was used as an example to illustrate how the system works. AID is a cytidine deaminase that can catalyze the reaction of deamination of cytidine in the context of DNA or RNA. When brought to the targeted site, AID changes a C base to U base. In dividing cells, this could lead to a C to T point mutation. Alternatively, the change of C to U could trigger cellular DNA repair pathways, mainly excision repair pathway, which will remove the mismatching U-G base-pair, and replace with a T-A, A-T, C-G, or G-C pair. As a result, a point mutation would be generated at the target C-G site. As excision repair pathway is present in most, if not all, somatic cells, recruitment of AID to the target site can correct a C-G base pair to others. In that case, if a C-G base pair is an underlying disease-causing genetic mutation in somatic tissues/cells, the above-described approach can be used to correct the mutation and thereby treat the disease.

By the same token, if an underlying disease causing genetic mutation is an A-T base pair at a specific site, one can use the same approach to recruit an adenosine deaminase to the specific site, where adenosine deaminase can correct the A-T base pair to others. Other effector enzymes are expected to generate other types of changes in base-pairing. A non-exhaustive list of examples of DNA/RNA modifying enzymes is detailed in Table 3.

TABLE 3
Examples of effector proteins that can be used in this invention

| | Genetic | Effector protein |
|---|---|---|
| Enzyme type | change | abbreviated |
| Cytidine | C→U/T | AID |
| deaminase | | APOBEC1 |
| | | APOBEC3A |
| | | APOBEC3B |
| | | APOBEC3C |
| | | APOBEC3D |
| | | APOBEC3F |
| | | APOBEC3G |
| | | APOBEC3H |
| Adenosine | A→I/G | ADA |
| deaminase | | ADAR1 |
| DNA Methyl | C→Met-C | Dnmt1 |
| transferase | | Dnmt3a |
| | | Dnmt3b |
| Demethylase | Met-C→C | Tet1 |

Effector protein full names:
AID: activation induced cytidine deaminase, a.k.a AICDA
APOBEC1: apolipoprotein B mRNA editing enzyme, catalytic polypeptide-like 1.
APOBEC3A: apolipoprotein B mRNA editing enzyme, catalytic polypeptide-like 3A
APOBEC3B: apolipoprotein B mRNA editing enzyme, catalytic polypeptide-like 3B
APOBEC3C: apolipoprotein B mRNA editing enzyme, catalytic polypeptide-like 3C
APOBEC3D: apolipoprotein B mRNA editing enzyme, catalytic polypeptide-like 3D
APOBEC3F: apolipoprotein B mRNA editing enzyme, catalytic polypeptide-like 3F
APOBEC3G: apolipoprotein B mRNA editing enzyme, catalytic polypeptide-like 3G
APOBEC3H: apolipoprotein B mRNA editing enzyme, catalytic polypeptide-like 3H
ADA: adenosine deaminase
ADAR1: adenosine deaminase acting on RNA 1
Dnmt1: DNA (cytosine-5-)-methyltransferase 1
Dnmt3a: DNA (cytosine-5-)-methyltransferase 3 alpha
Dnmt3b: DNA (cytosine-5-)-methyltransferase 3 beta
Tet1: methylcytosine dioxygenase

The above-described three specific components constitute the technological platform. Each component could be chosen from the list in Table 1-3 respectively to achieve a specific therapeutic/utility goal.

In one example, a CasRcure system was constructed using (i) dCas9 from S. pyogenes as the sequence targeting protein, (ii) an RNA scaffold containing a guide RNA sequence, a CRISPR RNA motif, and a MS2 operator motif, and (iii) an effector fusion containing a human AID fusing to MS2 operator binding protein MCP. The sequences for the components are listed below:

S. pyogenes dCas9-2xUGI protein sequence
(SEQ ID NO: 64)

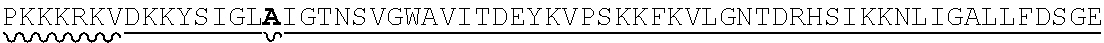

TAEATRLKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNSDVDK
LFIQLVQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLSDILRVN
TEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKD
NREKIEKILTFRIPYYVGPLARGNSRFAWMTRKSEETITPWNFEEVVDKGASAQSFIERMTNF
DKNLPNEKVLPKHSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEENEDILEDIVLTLT
LFEDREMIEERLKTYAHLFDDKVMKQLKRRRYTGWGRLSRKLINGIRDKQSGKTILDFLKSDG
FANRNFMQLIHDDSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVENTQLQNEKLYL

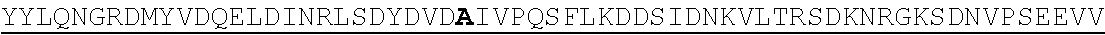

KKMKNYWRQLLNAKLITQRKFDNLTKAERGGLSELDKAGFIKRQLVETRQITKHVAQILDSRM
NTKYDENDKLIREVKVITLKSKLVSDFRKDFQFYKVREINNYHHAHDAYLNAVVGTALIKKYP
KLESEFVYGDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKLIARKKDWDP
KKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQ
KQLFVEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGDSGGSGGSGGSTN
LSDIIEKETGKQLVIQESILMLPEEVEEVIGNKPESDILVHTAYDESTDENVMLLTSDAPEYK
PWALVIQDSNGENKIKMLSGGSGGSGGSTNLSDIIEKETGKQLVIQESILMLPEEVEEVIGNK

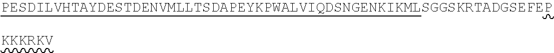

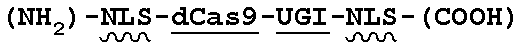

(Residues underlined: D10A, H840A active site mutants)
S. pyogenes nCas9_{D10A}-2xUGI protein sequence
(SEQ ID NO: 65)

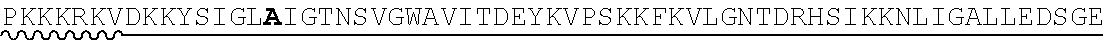

TAEATRLKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNSDVDK
LFIQLVQTYNQLFEENPINASGVDAKAILSARLSKSRRLENLIAQLPGEKKNGLFGNLIALSL
GLTPNEKSNEDLAEDAKLQLSKDTYDDDLDNLLAQIGDQYADLFLAAKNLSDAILLSDILRVN
TEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKD
NREKIEKILTFRIPYYVGPLARGNSRFAWMTRKSEETITPWNFEEVVDKGASAQSFIERMTNF
DKNLPNEKVLPKHSLLYEYFTVYNELTKVKYVTEGMRKPAELSGEQKKAIVDLLEKTNRKVTV
KQLKEDYFKKIECEDSVEISGVEDRFNASLGTYHDLLKIIKDKDFLDNEENEDILEDIVLTLT
LFEDREMIEERLKTYAHLFDDKVMKQLKRRRYTGWGRLSRKLINGIRDKQSGKTILDFLKSDG
FANRNFMQLIHDDSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVENTQLQNEKLYL
YYLQNGRDMYVDQELDINRLSDYDVDHIVPQSFLKDDSIDNKVLTRSDKNRGKSDNVPSEEVV
KKMKNYWRQLLNAKLITQRKFDNLTKAERGGLSELDKAGFIKRQLVETRQITKHVAQILDSRM
NTKYDENDKLIREVKVITLKSKLVSDFRKDFQFYKVREINNYHHAHDAYLNAVVGTALIKKYP
KLESEFVYGDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKLIARKKDWDP
KKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQ
KQLFVEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGDSGGSGGSGGSTN
LSDIIEKETGKQLVIQESILMLPEEVEEVIGNKPESDILVHTAYDESTDENVMLLTSDAPEYK
PWALVIQDSNGENKIKMLSGGSGGSGGSTNLSDIIEKETGKQLVIQESILMLPEEVEEVIGNK

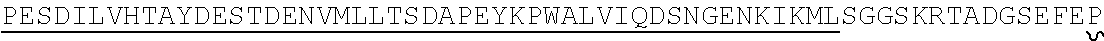

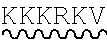

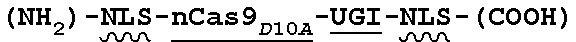

(Residues underlined: D10A active site mutant)
Codon optimized cDNA encoding catalytically dead Cas9-2xUGI sequence 1
(SEQ ID NO: 66):

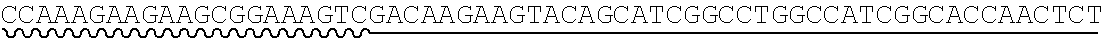

GTGGGCTGGGCCGTGATCACCGACGAGTACAAGGTGCCCAGCAAGAAATTCAAGGTGCTGGGC
AACACCGACCGGCACAGCATCAAGAAGAACCTGATCGGAGCCCTGCTGTTCGACAGCGGCGAA
ACAGCCGAGGCCACCCGGCTGAAGAGAACCGCCAGAAGAAGATACACCAGACGGAAGAACCGG
ATCTGCTATCTGCAAGAGATCTTCAGCAACGAGATGGCCAAGGTGGACGACAGCTTCTTCCAC
AGACTGGAAGAGTCCTTCCTGGTGGAAGAGGATAAGAAGCACGAGCGGCACCCCATCTTCGGC
AACATCGTGGACGAGGTGGCCTACCACGAGAAGTACCCCACCATCTACCACCTGAGAAAGAAA
CTGGTGGACAGCACCGACAAGGCCGACCTGCGGCTGATCTATCTGGCCCTGGCCCACATGATC
AAGTTCCGGGGCCACTTCCTGATCGAGGGCGACCTGAACCCCGACAACAGCGACGTGGACAAG
CTGTTCATCCAGCTGGTGCAGACCTACAACCAGCTGTTCGAGGAAAACCCCATCAACGCCAGC
GGCGTGGACGCCAAGGCCATCCTGTCTGCCAGACTGAGCAAGAGCAGACGGCTGGAAAATCTG
ATCGCCCAGCTGCCCGGCGAGAAGAAGAATGGCCTGTTCGGAAACCTGATTGCCCTGAGCCTG
GGCCTGACCCCCAACTTCAAGAGCAACTTCGACCTGGCCGAGGATGCCAAACTGCAGCTGAGC
AAGGACACCTACGACGACGACCTGGACAACCTGCTGGCCCAGATCGGCGACCAGTACGCCGAC
CTGTTTCTGGCCGCCAAGAACCTGTCCGACGCCATCCTGCTGAGCGACATCCTGAGAGTGAAC
ACCGAGATCACCAAGGCCCCCCTGAGCGCCTCTATGATCAAGAGATACGACGAGCACCACCAG
GACCTGACCCTGCTGAAAGCTCTCGTGCGGCAGCAGCTGCCTGAGAAGTACAAAGAGATTTTC
TTCGACCAGAGCAAGAACGGCTACGCCGGCTACATTGACGGCGGAGCCAGCCAGGAAGAGTTC
TACAAGTTCATCAAGCCCATCCTGGAAAAGATGGACGGCACCGAGGAACTGCTCGTGAAGCTG
AACAGAGAGGACCTGCTGCGGAAGCAGCGGACCTTCGACAACGGCAGCATCCCCCACCAGATC
CACCTGGGAGAGCTGCACGCCATTCTGCGGCGGCAGGAAGATTTTTACCCATTCCTGAAGGAC
AACCGGGAAAAGATCGAGAAGATCCTGACCTTCCGCATCCCCTACTACGTGGGCCCTCTGGCC
AGGGGAAACAGCAGATTCGCCTGGATGACCAGAAAGAGCGAGGAAACCATCACCCCCTGGAAC
TTCGAGGAAGTGGTGGACAAGGGCGCTTCCGCCCAGAGCTTCATCGAGCGGATGACCAACTTC
GATAAGAACCTGCCCAACGAGAAGGTGCTGCCCAAGCACAGCCTGCTGTACGAGTACTTCACC
GTGTATAACGAGCTGACCAAAGTGAAATACGTGACCGAGGGAATGAGAAAGCCCGCCTTCCTG
AGCGGCGAGCAGAAAAAGGCCATCGTGGACCTGCTGTTCAAGACCAACCGGAAAGTGACCGTG
AAGCAGCTGAAAGAGGACTACTTCAAGAAAATCGAGTGCTTCGACTCCGTGGAAATCTCCGGC
GTGGAAGATCGGTTCAACGCCTCCCTGGGCACATACCACGATCTGCTGAAAATTATCAAGGAC
AAGGACTTCCTGGACAATGAGGAAAACGAGGACATTCTGGAAGATATCGTGCTGACCCTGACA
CTGTTTGAGGACAGAGAGATGATCGAGGAACGGCTGAAAACCTATGCCCACCTGTTCGACGAC
AAAGTGATGAAGCAGCTGAAGCGGCGGAGATACACCGGCTGGGGCAGGCTGAGCCGGAAGCTG
ATCAACGGCATCCGGGACAAGCAGTCCGGCAAGACAATCCTGGATTTCCTGAAGTCCGACGGC
TTCGCCAACAGAAACTTCATGCAGCTGATCCACGACGACAGCCTGACCTTTAAAGAGGACATC
CAGAAAGCCCAGGTGTCCGGCCAGGGCGATAGCCTGCACGAGCACATTGCCAATCTGGCCGGC
AGCCCCGCCATTAAGAAGGGCATCCTGCAGACAGTGAAGGTGGTGGACGAGCTCGTGAAAGTG
ATGGGCCGGCACAAGCCCGAGAACATCGTGATCGAAATGGCCAGAGAGAACCAGACCACCCAG
AAGGGACAGAAGAACAGCCGCGAGAGAATGAAGCGGATCGAAGAGGGCATCAAAGAGCTGGGC
AGCCAGATCCTGAAAGAACACCCCGTGGAAAACACCCAGCTGCAGAACGAGAAGCTGTACCTG
TACTACCTGCAGAATGGGCGGGATATGTACGTGGACCAGGAACTGGACATCAACCGGCTGTCC
GACTACGATGTGGACGCCATCGTGCCTCAGAGCTTTCTGAAGGACGACTCCATCGACAACAAG
GTGCTGACCAGAAGCGACAAGAACCGGGGCAAGAGCGACAACGTGCCCTCCGAAGAGGTCGTG
AAGAAGATGAAGAACTACTGGCGGCAGCTGCTGAACGCCAAGCTGATTACCCAGAGAAAGTTC
GACAATCTGACCAAGGCCGAGAGAGGCGGCCTGAGCGAACTGGATAAGGCCGGCTTCATCAAG
AGACAGCTGGTGGAAACCCGGCAGATCACAAAGCACGTGGCACAGATCCTGGACTCCCGGATG
AACACTAAGTACGACGAGAATGACAAGCTGATCCGGGAAGTGAAAGTGATCACCCTGAAGTCC
AAGCTGGTGTCCGATTTCCGGAAGGATTTCCAGTTTTACAAAGTGCGCGAGATCAACAACTAC
CACCACGCCCACGACGCCTACCTGAACGCCGTCGTGGGAACCGCCCTGATCAAAAAGTACCCT
AAGCTGGAAAGCGAGTTCGTGTACGGCGACTACAAGGTGTACGACGTGCGGAAGATGATCGCC
AAGAGCGAGCAGGAAATCGGCAAGGCTACCGCCAAGTACTTCTTCTACAGCAACATCATGAAC
TTTTTCAAGACCGAGATTACCCTGGCCAACGGCGAGATCCGGAAGCGGCCTCTGATCGAGACA
AACGGCGAAACCGGGGAGATCGTGTGGGATAAGGGCCGGGATTTTGCCACCGTGCGGAAAGTG
CTGAGCATGCCCCAAGTGAATATCGTGAAAAAGACCGAGGTGCAGACAGGCGGCTTCAGCAAA
GAGTCTATCCTGCCCAAGAGGAACAGCGATAAGCTGATCGCCAGAAAGAAGGACTGGGACCCT
AAGAAGTACGGCGGCTTCGACAGCCCCACCGTGGCCTATTCTGTGCTGGTGGTGGCCAAAGTG
GAAAAGGGCAAGTCCAAGAAACTGAAGAGTGTGAAAGAGCTGCTGGGGATCACCATCATGGAA
AGAAGCAGCTTCGAGAAGAATCCCATCGACTTTCTGGAAGCCAAGGGCTACAAAGAAGTGAAA
AAGGACCTGATCATCAAGCTGCCTAAGTACTCCCTGTTCGAGCTGGAAAACGGCCGGAAGAGA
ATGCTGGCCTCTGCCGGCGAACTGCAGAAGGGAAACGAACTGGCCCTGCCCTCCAAATATGTG
AACTTCCTGTACCTGGCCAGCCACTATGAGAAGCTGAAGGGCTCCCCCGAGGATAATGAGCAG
AAACAGCTGTTTGTGGAACAGCACAAGCACTACCTGGACGAGATCATCGAGCAGATCAGCGAG
TTCTCCAAGAGAGTGATCCTGGCCGACGCTAATCTGGACAAAGTGCTGTCCGCCTACAACAAG
CACCGGGATAAGCCCATCAGAGAGCAGGCCGAGAATATCATCCACCTGTTTACCCTGACCAAT
CTGGGAGCCCCTGCCGCCTTCAAGTACTTTGACACCACCATCGACCGGAAGAGGTACACCAGC
ACCAAAGAGGTGCTGGACGCCACCCTGATCCACCAGAGCATCACCGGCCTGTACGAGACACGG
ATCGACCTGTCTCAGCTGGGAGGTGACAGCGGCGGGAGCGGCGGGAGCGGGGGGAGCACTAAT
CTGAGCGACATCATTGAGAAGGAGACTGGGAAACAGCTGGTCATTCAGGAGTCCATCCTGATG
CTGCCTGAGGAGGTGGAGGAAGTGATCGGCAACAAGCCAGAGTCTGACATCCTGGTGCACACC
GCCTACGACGAGTCCACAGATGAGAATGTGATGCTGCTGACCTCTGACGCCCCCGAGTATAAG
CCTTGGGCCCTGGTCATCCAGGATTCTAACGGCGAGAATAAGATCAAGATGCTGAGCGGAGGA
TCCGGAGGATCTGGAGGCAGCACCAACCTGTCTGACATCATCGAGAAGGAGACAGGCAAGCAG
CTGGTCATCCAGGAGAGCATCCTGATGCTGCCCGAAGAAGTCGAAGAAGTGATCGGAAACAAG
CCTGAGAGCGATATCCTGGTCCATACCGCCTACGACGAGAGTACCGACGAAAATGTGATGCTG
CTGACATCCGACGCCCCAGAGTATAAGCCCTGGGCTCTGGTCATCCAGGATTCCAACGGAGAG

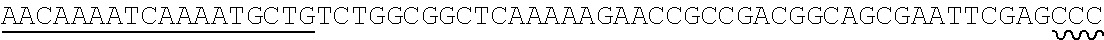

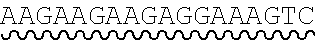

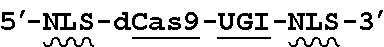

Codon optimized cDNA encoding catalytically dead Cas9-2xUGI sequence 2
(SEQ ID NO: 67)

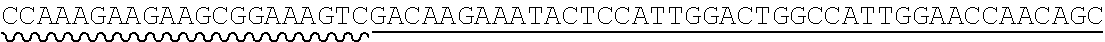

GTCGGATGGGCCGTGATCACCGACGAGTATAAAGTCCCCTCCAAGAAATTCAAGGTGCTGGGC
AATACCGACAGACATTCCATCAAGAAGAATCTGATCGGCGCTCTGCTCTTCGATTCCGGCGAG
ACCGCCGAAGCTACAAGACTGAAGAGAACAGCTAGAAGGAGATATACAAGAAGGAAGAATAGA
ATCTGTTACCTCCAAGAGATCTTCAGCAACGAGATGGCCAAAGTCGATGACAGCTTCTTCCAC
AGACTCGAAGAGAGCTTTCTCGTGGAGGAGGACAAGAAGCACGAGAGACACCCTATCTTCGGC
AACATCGTGGATGAGGTCGCCTATCATGAGAAATACCCCACCATCTACCATCTGAGGAAGAAA
CTCGTCGACTCCACCGATAAAGCCGATCTCAGACTGATCTATCTGGCTCTGGCCCATATGATC
AAGTTTAGGGGCCACTTTCTGATTGAGGGCGACCTCAACCCCGACAACTCCGATGTGGACAAA
CTCTTCATCCAGCTGGTCCAGACATACAACCAGCTGTTCGAGGAGAACCCTATTAACGCCTCC
GGCGTGGATGCCAAGGCTATTCTGAGCGCCAGACTGTCCAAATCTAGAAGGCTCGAAAACCTC
ATCGCTCAACTGCCCGGCGAGAAAAAGAACGGCCTCTTCGGCAATCTGATTGCCCTCTCTCTG
GGACTGACCCCTAATTTCAAATCCAACTTTGATCTGGCCGAGGACGCCAAACTGCAGCTCTCC
AAAGACACATACGACGACGATCTGGACAATCTGCTCGCTCAGATCGGAGACCAGTACGCCGAT
CTGTTTCTGGCCGCCAAGAACCTCTCCGATGCCATTCTGCTGAGCGACATTCTGAGGGTGAAC
ACAGAAATCACCAAGGCCCCTCTGTCCGCCAGCATGATCAAGAGGTATGACGAACACCATCAA
GACCTCACACTGCTGAAAGCCCTCGTGAGACAGCAACTCCCCGAAAAATACAAAGAGATCTTT
TTTGACCAGAGCAAAAATGGCTATGCCGGCTATATCGATGGCGGCGCTAGCCAAGAGGAGTTC
TACAAATTCATTAAGCCCATTCTGGAGAAAATGGATGGCACAGAGGAACTGCTGGTGAAGCTG
AATAGGGAGGATCTGCTGAGAAAGCAAAGGACATTCGACAACGGCTCCATCCCCCACCAGATT
CATCTGGGCGAGCTCCATGCCATTCTGAGAAGGCAAGAGGACTTCTATCCCTTCCTCAAAGAC
AATAGAGAGAAAATCGAAAAGATTCTGACCTTCAGAATCCCTTATTATGTCGGCCCCCTCGCT
AGAGGAAACTCTAGATTCGCTTGGATGACAAGAAAGTCCGAGGAGACAATCACCCCTTGGAAC
TTTGAGGAAGTGGTGGACAAGGGAGCCAGCGCCCAGAGCTTCATTGAAAGGATGACAAATTTT
GACAAGAACCTCCCCAACGAGAAAGTGCTGCCTAAGCACTCTCTGCTGTACGAGTACTTCACA
GTCTATAATGAGCTGACCAAAGTGAAGTATGTCACCGAAGGCATGAGGAAACCCGCTTTCCTC
AGCGGCGAGCAGAAGAAGGCCATCGTCGATCTGCTGTTTAAGACCAATAGAAAAGTCACCGTC
AAACAGCTGAAGGAAGATTACTTCAAGAAAATTGAGTGCTTCGACTCCGTGGAAATCAGCGGC
GTCGAGGATAGATTTAACGCTTCTCTGGGCACATACCATGATCTGCTGAAGATCATCAAAGAC
AAGGATTTTCTCGACAACGAAGAGAACGAGGACATCCTCGAGGATATCGTGCTGACACTGACC
CTCTTCGAGGATAGAGAAATGATCGAGGAGAGGCTCAAGACATATGCCCACCTCTTCGACGAC
AAGGTGATGAAACAACTGAAGAGAAGAAGATACACCGGCTGGGGAAGACTCTCTAGAAAGCTC
ATCAATGGCATTAGGGACAAGCAAAGCGGAAAGACCATTCTCGACTTCCTCAAGTCCGACGGC
TTTGCCAATAGGAACTTTATGCAGCTCATCCATGACGATTCTCTGACATTCAAGGAGGACATC
CAGAAGGCCCAAGTGAGCGGACAAGGAGATTCCCTCCATGAACATATCGCTAACCTCGCCGGA
TCCCCCGCCATTAAAAAGGGAATCCTCCAAACAGTGAAGGTCGTGGATGAGCTGGTCAAAGTG
ATGGGCAGACACAAACCCGAGAACATTGTCATCGAGATGGCCAGAGAGAACCAGACCACCCAA
AAAGGACAGAAGAACTCCAGAGAAAGGATGAAAAGAATCGAGGAAGGAATCAAGGAACTCGGC
TCCCAGATCCTCAAGGAGCATCCCGTGGAGAATACCCAGCTGCAGAATGAGAAACTGTACCTC
TACTACCTCCAGAATGGAAGGGACATGTACGTCGACCAAGAACTCGACATCAACAGACTGAGC
GACTACGATGTCGACGCTATCGTGCCCCAGAGCTTTCTGAAAGACGACTCCATCGATAACAAG
GTCCTCACAAGATCCGACAAGAACAGAGGCAAGAGCGACAACGTCCCCTCCGAAGAGGTGGTG
AAAAAGATGAAGAACTACTGGAGGCAGCTGCTGAACGCCAAACTCATCACCCAGAGGAAGTTC
GATAATCTGACCAAAGCCGAAAGAGGAGGACTGTCCGAACTGGACAAAGCCGGCTTTATCAAG
AGGCAGCTGGTGGAAACCAGACAGATCACCAAACATGTCGCCCAAATTCTGGACTCTAGAATG
AACACCAAGTACGACGAAAATGACAAGCTGATTAGAGAAGTGAAGGTCATCACCCTCAAGAGC
AAGCTGGTCTCCGATTTTAGAAAGGATTTCCAATTCTACAAGGTCAGAGAGATCAATAATTAC
CACCATGCCCACGATGCCTATCTGAACGCCGTGGTGGGAACAGCCCTCATCAAGAAGTACCCT
AAGCTGGAAAGCGAGTTCGTGTATGGAGATTATAAAGTCTACGATGTGAGGAAGATGATTGCC
AAGTCCGAGCAAGAGATCGGCAAGGCCACCGCTAAATACTTCTTTTATTCCAACATCATGAAC
TTCTTTAAAACCGAGATCACACTCGCTAATGGCGAGATTAGGAAGAGACCTCTGATCGAGACA
AACGGCGAGACCGGCGAGATCGTCTGGGACAAGGGCAGAGATTTCGCCACCGTGAGAAAGGTG
CTCTCCATGCCTCAAGTGAACATCGTGAAAAAGACCGAGGTGCAGACCGGCGGCTTCTCCAAG
GAGTCCATTCTGCCCAAAAGGAACTCCGACAAGCTCATCGCTAGAAAGAAGGATTGGGATCCT
AAGAAATACGGCGGATTTGACTCCCCTACAGTCGCTTACAGCGTGCTCGTGGTGGCCAAGGTC
GAGAAGGGCAAGTCCAAGAAGCTGAAGTCCGTGAAGGAGCTGCTGGGAATCACAATCATGGAG
AGGTCCTCCTTCGAGAAGAACCCCATCGATTTTCTGGAGGCCAAGGGCTACAAAGAGGTGAAG
AAAGATCTGATCATTAAGCTGCCCAAATATTCCCTCTTCGAGCTGGAGAACGGAAGAAAAAGG
ATGCTGGCCTCCGCTGGCGAACTGCAGAAGGGAAACGAGCTCGCTCTCCCCAGCAAGTACGTC
AACTTCCTCTACCTCGCCAGCCACTACGAGAAACTGAAGGGATCCCCCGAGGACAATGAGCAG
AAGCAGCTCTTCGTGGAGCAGCACAAGCATTACCTCGATGAGATCATCGAGCAGATCTCCGAA
TTCAGCAAGAGGGTCATTCTGGCTGACGCCAACCTCGATAAGGTCCTCAGCGCTTACAACAAG
CACAGAGATAAGCCCATTAGGGAGCAAGCCGAAAATATCATCCATCTGTTTACACTGACAAAT
CTGGGCGCCCCCGCCGCTTTTAAGTACTTCGATACCACCATCGATAGAAAGAGGTACACCTCC
ACAAAAGAGGTGCTGGATGCTACCCTCATCCATCAGTCCATTACCGGACTCTACGAGACCAGA
ATTGATCTCTCCCAGCTGGGAGGAGATAGCGGCGGGAGCGGCGGGAGCGGGGGGAGCACTAAT
CTGAGCGACATCATTGAGAAGGAGACTGGGAAACAGCTGGTCATTCAGGAGTCCATCCTGATG
CTGCCTGAGGAGGTGGAGGAAGTGATCGGCAACAAGCCAGAGTCTGACATCCTGGTGCACACC
GCCTACGACGAGTCCACAGATGAGAATGTGATGCTGCTGACCTCTGACGCCCCCGAGTATAAG
CCTTGGGCCCTGGTCATCCAGGATTCTAACGGCGAGAATAAGATCAAGATGCTGAGCGGAGGA
TCCGGAGGATCTGGAGGCAGCACCAACCTGTCTGACATCATCGAGAAGGAGACAGGCAAGCAG
CTGGTCATCCAGGAGAGCATCCTGATGCTGCCCGAAGAAGTCGAAGAAGTGATCGGAAACAAG
CCTGAGAGCGATATCCTGGTCCATACCGCCTACGACGAGAGTACCGACGAAAATGTGATGCTG
CTGACATCCGACGCCCCAGAGTATAAGCCCTGGGCTCTGGTCATCCAGGATTCCAACGGAGAG

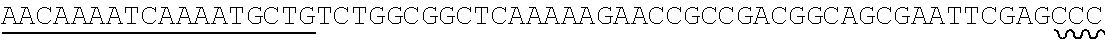

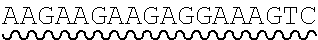

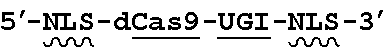

Codon optimized cDNA encoding nCas9_{D10A}-2xUGI sequence 1
(SEQ ID NO: 68)

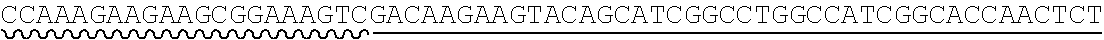

GTGGGCTGGGCCGTGATCACCGACGAGTACAAGGTGCCCAGCAAGAAATTCAAGGTGCTGGGC
AACACCGACCGGCACAGCATCAAGAAGAACCTGATCGGAGCCCTGCTGTTCGACAGCGGCGAA
ACAGCCGAGGCCACCCGGCTGAAGAGAACCGCCAGAAGAAGATACACCAGACGGAAGAACCGG
ATCTGCTATCTGCAAGAGATCTTCAGCAACGAGATGGCCAAGGTGGACGACAGCTTCTTCCAC
AGACTGGAAGAGTCCTTCCTGGTGGAAGAGGATAAGAAGCACGAGCGGCACCCCATCTTCGGC
AACATCGTGGACGAGGTGGCCTACCACGAGAAGTACCCCACCATCTACCACCTGAGAAAGAAA
CTGGTGGACAGCACCGACAAGGCCGACCTGCGGCTGATCTATCTGGCCCTGGCCCACATGATC
AAGTTCCGGGGCCACTTCCTGATCGAGGGCGACCTGAACCCCGACAACAGCGACGTGGACAAG
CTGTTCATCCAGCTGGTGCAGACCTACAACCAGCTGTTCGAGGAAAACCCCATCAACGCCAGC
GGCGTGGACGCCAAGGCCATCCTGTCTGCCAGACTGAGCAAGAGCAGACGGCTGGAAAATCTG
ATCGCCCAGCTGCCCGGCGAGAAGAAGAATGGCCTGTTCGGAAACCTGATTGCCCTGAGCCTG
GGCCTGACCCCCAACTTCAAGAGCAACTTCGACCTGGCCGAGGATGCCAAACTGCAGCTGAGC
AAGGACACCTACGACGACGACCTGGACAACCTGCTGGCCCAGATCGGCGACCAGTACGCCGAC
CTGTTTCTGGCCGCCAAGAACCTGTCCGACGCCATCCTGCTGAGCGACATCCTGAGAGTGAAC
ACCGAGATCACCAAGGCCCCCCTGAGCGCCTCTATGATCAAGAGATACGACGAGCACCACCAG
GACCTGACCCTGCTGAAAGCTCTCGTGCGGCAGCAGCTGCCTGAGAAGTACAAAGAGATTTTC
TTCGACCAGAGCAAGAACGGCTACGCCGGCTACATTGACGGCGGAGCCAGCCAGGAAGAGTTC
TACAAGTTCATCAAGCCCATCCTGGAAAAGATGGACGGCACCGAGGAACTGCTCGTGAAGCTG
AACAGAGAGGACCTGCTGCGGAAGCAGCGGACCTTCGACAACGGCAGCATCCCCCACCAGATC
CACCTGGGAGAGCTGCACGCCATTCTGCGGCGGCAGGAAGATTTTTACCCATTCCTGAAGGAC
AACCGGGAAAAGATCGAGAAGATCCTGACCTTCCGCATCCCCTACTACGTGGGCCCTCTGGCC
AGGGGAAACAGCAGATTCGCCTGGATGACCAGAAAGAGCGAGGAAACCATCACCCCCTGGAAC
TTCGAGGAAGTGGTGGACAAGGGCGCTTCCGCCCAGAGCTTCATCGAGCGGATGACCAACTTC
GATAAGAACCTGCCCAACGAGAAGGTGCTGCCCAAGCACAGCCTGCTGTACGAGTACTTCACC
GTGTATAACGAGCTGACCAAAGTGAAATACGTGACCGAGGGAATGAGAAAGCCCGCCTTCCTG
AGCGGCGAGCAGAAAAAGGCCATCGTGGACCTGCTGTTCAAGACCAACCGGAAAGTGACCGTG
AAGCAGCTGAAAGAGGACTACTTCAAGAAAATCGAGTGCTTCGACTCCGTGGAAATCTCCGGC
GTGGAAGATCGGTTCAACGCCTCCCTGGGCACATACCACGATCTGCTGAAAATTATCAAGGAC
AAGGACTTCCTGGACAATGAGGAAAACGAGGACATTCTGGAAGATATCGTGCTGACCCTGACA
CTGTTTGAGGACAGAGAGATGATCGAGGAACGGCTGAAAACCTATGCCCACCTGTTCGACGAC
AAAGTGATGAAGCAGCTGAAGCGGCGGAGATACACCGGCTGGGGCAGGCTGAGCCGGAAGCTG
ATCAACGGCATCCGGGACAAGCAGTCCGGCAAGACAATCCTGGATTTCCTGAAGTCCGACGGC
TTCGCCAACAGAAACTTCATGCAGCTGATCCACGACGACAGCCTGACCTTTAAAGAGGACATC
CAGAAAGCCCAGGTGTCCGGCCAGGGCGATAGCCTGCACGAGCACATTGCCAATCTGGCCGGC
AGCCCCGCCATTAAGAAGGGCATCCTGCAGACAGTGAAGGTGGTGGACGAGCTCGTGAAAGTG
ATGGGCCGGCACAAGCCCGAGAACATCGTGATCGAAATGGCCAGAGAGAACCAGACCACCCAG
AAGGGACAGAAGAACAGCCGCGAGAGAATGAAGCGGATCGAAGAGGGCATCAAAGAGCTGGGC
AGCCAGATCCTGAAAGAACACCCCGTGGAAAACACCCAGCTGCAGAACGAGAAGCTGTACCTG
TACTACCTGCAGAATGGGCGGGATATGTACGTGGACCAGGAACTGGACATCAACCGGCTGTCC
GACTACGATGTGGACCATATCGTGCCTCAGAGCTTTCTGAAGGACGACTCCATCGACAACAAG
GTGCTGACCAGAAGCGACAAGAACCGGGGCAAGAGCGACAACGTGCCCTCCGAAGAGGTCGTG
AAGAAGATGAAGAACTACTGGCGGCAGCTGCTGAACGCCAAGCTGATTACCCAGAGAAAGTTC
GACAATCTGACCAAGGCCGAGAGAGGCGGCCTGAGCGAACTGGATAAGGCCGGCTTCATCAAG
AGACAGCTGGTGGAAACCCGGCAGATCACAAAGCACGTGGCACAGATCCTGGACTCCCGGATG
AACACTAAGTACGACGAGAATGACAAGCTGATCCGGGAAGTGAAAGTGATCACCCTGAAGTCC
AAGCTGGTGTCCGATTTCCGGAAGGATTTCCAGTTTTACAAAGTGCGCGAGATCAACAACTAC
CACCACGCCCACGACGCCTACCTGAACGCCGTCGTGGGAACCGCCCTGATCAAAAAGTACCCT
AAGCTGGAAAGCGAGTTCGTGTACGGCGACTACAAGGTGTACGACGTGCGGAAGATGATCGCC
AAGAGCGAGCAGGAAATCGGCAAGGCTACCGCCAAGTACTTCTTCTACAGCAACATCATGAAC
TTTTTCAAGACCGAGATTACCCTGGCCAACGGCGAGATCCGGAAGCGGCCTCTGATCGAGACA
AACGGCGAAACCGGGGAGATCGTGTGGGATAAGGGCCGGGATTTTGCCACCGTGCGGAAAGTG
CTGAGCATGCCCCAAGTGAATATCGTGAAAAAGACCGAGGTGCAGACAGGCGGCTTCAGCAAA
GAGTCTATCCTGCCCAAGAGGAACAGCGATAAGCTGATCGCCAGAAAGAAGGACTGGGACCCT
AAGAAGTACGGCGGCTTCGACAGCCCCACCGTGGCCTATTCTGTGCTGGTGGTGGCCAAAGTG
GAAAAGGGCAAGTCCAAGAAACTGAAGAGTGTGAAAGAGCTGCTGGGGATCACCATCATGGAA
AGAAGCAGCTTCGAGAAGAATCCCATCGACTTTCTGGAAGCCAAGGGCTACAAAGAAGTGAAA
AAGGACCTGATCATCAAGCTGCCTAAGTACTCCCTGTTCGAGCTGGAAAACGGCCGGAAGAGA
ATGCTGGCCTCTGCCGGCGAACTGCAGAAGGGAAACGAACTGGCCCTGCCCTCCAAATATGTG
AACTTCCTGTACCTGGCCAGCCACTATGAGAAGCTGAAGGGCTCCCCCGAGGATAATGAGCAG
AAACAGCTGTTTGTGGAACAGCACAAGCACTACCTGGACGAGATCATCGAGCAGATCAGCGAG
TTCTCCAAGAGAGTGATCCTGGCCGACGCTAATCTGGACAAAGTGCTGTCCGCCTACAACAAG
CACCGGGATAAGCCCATCAGAGAGCAGGCCGAGAATATCATCCACCTGTTTACCCTGACCAAT
CTGGGAGCCCCTGCCGCCTTCAAGTACTTTGACACCACCATCGACCGGAAGAGGTACACCAGC
ACCAAAGAGGTGCTGGACGCCACCCTGATCCACCAGAGCATCACCGGCCTGTACGAGACACGG
ATCGACCTGTCTCAGCTGGGAGGTGACAGCGGCGGGAGCGGCGGGAGCGGGGGGAGCACTAAT
CTGAGCGACATCATTGAGAAGGAGACTGGGAAACAGCTGGTCATTCAGGAGTCCATCCTGATG
CTGCCTGAGGAGGTGGAGGAAGTGATCGGCAACAAGCCAGAGTCTGACATCCTGGTGCACACC
GCCTACGACGAGTCCACAGATGAGAATGTGATGCTGCTGACCTCTGACGCCCCCGAGTATAAG
CCTTGGGCCCTGGTCATCCAGGATTCTAACGGCGAGAATAAGATCAAGATGCTGAGCGGAGGA
TCCGGAGGATCTGGAGGCAGCACCAACCTGTCTGACATCATCGAGAAGGAGACAGGCAAGCAG
CTGGTCATCCAGGAGAGCATCCTGATGCTGCCCGAAGAAGTCGAAGAAGTGATCGGAAACAAG
CCTGAGAGCGATATCCTGGTCCATACCGCCTACGACGAGAGTACCGACGAAAATGTGATGCTG
CTGACATCCGACGCCCCAGAGTATAAGCCCTGGGCTCTGGTCATCCAGGATTCCAACGGAGAG

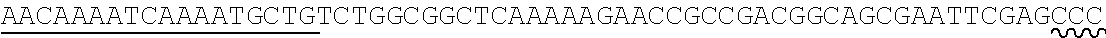

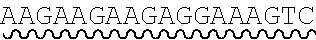

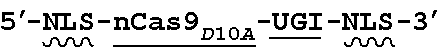

Codon optimized cDNA encoding nCas9_{D10A}-2xUGI sequence 2
(SEQ ID NO: 69):

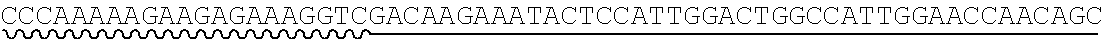

GTCGGATGGGCCGTGATCACCGACGAGTATAAAGTCCCCTCCAAGAAATTCAAGGTGCTGGGC
AATACCGACAGACATTCCATCAAGAAGAATCTGATCGGCGCTCTGCTCTTCGATTCCGGCGAG
ACCGCCGAAGCTACAAGACTGAAGAGAACAGCTAGAAGGAGATATACAAGAAGGAAGAATAGA
ATCTGTTACCTCCAAGAGATCTTCAGCAACGAGATGGCCAAAGTCGATGACAGCTTCTTCCAC
AGACTCGAAGAGAGCTTTCTCGTGGAGGAGGACAAGAAGCACGAGAGACACCCTATCTTCGGC
AACATCGTGGATGAGGTCGCCTATCATGAGAAATACCCCACCATCTACCATCTGAGGAAGAAA
CTCGTCGACTCCACCGATAAAGCCGATCTCAGACTGATCTATCTGGCTCTGGCCCATATGATC
AAGTTTAGGGGCCACTTTCTGATTGAGGGCGACCTCAACCCCGACAACTCCGATGTGGACAAA
CTCTTCATCCAGCTGGTCCAGACATACAACCAGCTGTTCGAGGAGAACCCTATTAACGCCTCC
GGCGTGGATGCCAAGGCTATTCTGAGCGCCAGACTGTCCAAATCTAGAAGGCTCGAAAACCTC
ATCGCTCAACTGCCCGGCGAGAAAAAGAACGGCCTCTTCGGCAATCTGATTGCCCTCTCTCTG
GGACTGACCCCTAATTTCAAATCCAACTTTGATCTGGCCGAGGACGCCAAACTGCAGCTCTCC
AAAGACACATACGACGACGATCTGGACAATCTGCTCGCTCAGATCGGAGACCAGTACGCCGAT
CTGTTTCTGGCCGCCAAGAACCTCTCCGATGCCATTCTGCTGAGCGACATTCTGAGGGTGAAC
ACAGAAATCACCAAGGCCCCTCTGTCCGCCAGCATGATCAAGAGGTATGACGAACACCATCAA
GACCTCACACTGCTGAAAGCCCTCGTGAGACAGCAACTCCCCGAAAAATACAAAGAGATCTTT
TTTGACCAGAGCAAAAATGGCTATGCCGGCTATATCGATGGCGGCGCTAGCCAAGAGGAGTTC
TACAAATTCATTAAGCCCATTCTGGAGAAAATGGATGGCACAGAGGAACTGCTGGTGAAGCTG
AATAGGGAGGATCTGCTGAGAAAGCAAAGGACATTCGACAACGGCTCCATCCCCCACCAGATT
CATCTGGGCGAGCTCCATGCCATTCTGAGAAGGCAAGAGGACTTCTATCCCTTCCTCAAAGAC
AATAGAGAGAAAATCGAAAAGATTCTGACCTTCAGAATCCCTTATTATGTCGGCCCCCTCGCT
AGAGGAAACTCTAGATTCGCTTGGATGACAAGAAAGTCCGAGGAGACAATCACCCCTTGGAAC
TTTGAGGAAGTGGTGGACAAGGGAGCCAGCGCCCAGAGCTTCATTGAAAGGATGACAAATTTT
GACAAGAACCTCCCCAACGAGAAAGTGCTGCCTAAGCACTCTCTGCTGTACGAGTACTTCACA
GTCTATAATGAGCTGACCAAAGTGAAGTATGTCACCGAAGGCATGAGGAAACCCGCTTTCCTC
AGCGGCGAGCAGAAGAAGGCCATCGTCGATCTGCTGTTTAAGACCAATAGAAAAGTCACCGTC
AAACAGCTGAAGGAAGATTACTTCAAGAAAATTGAGTGCTTCGACTCCGTGGAAATCAGCGGC
GTCGAGGATAGATTTAACGCTTCTCTGGGCACATACCATGATCTGCTGAAGATCATCAAAGAC
AAGGATTTTCTCGACAACGAAGAGAACGAGGACATCCTCGAGGATATCGTGCTGACACTGACC
CTCTTCGAGGATAGAGAAATGATCGAGGAGAGGCTCAAGACATATGCCCACCTCTTCGACGAC
AAGGTGATGAAACAACTGAAGAGAAGAAGATACACCGGCTGGGGAAGACTCTCTAGAAAGCTC
ATCAATGGCATTAGGGACAAGCAAAGCGGAAAGACCATTCTCGACTTCCTCAAGTCCGACGGC
TTTGCCAATAGGAACTTTATGCAGCTCATCCATGACGATTCTCTGACATTCAAGGAGGACATC
CAGAAGGCCCAAGTGAGCGGACAAGGAGATTCCCTCCATGAACATATCGCTAACCTCGCCGGA
TCCCCCGCCATTAAAAAGGGAATCCTCCAAACAGTGAAGGTCGTGGATGAGCTGGTCAAAGTG
ATGGGCAGACACAAACCCGAGAACATTGTCATCGAGATGGCCAGAGAGAACCAGACCACCCAA
AAAGGACAGAAGAACTCCAGAGAAAGGATGAAAAGAATCGAGGAAGGAATCAAGGAACTCGGC
TCCCAGATCCTCAAGGAGCATCCCGTGGAGAATACCCAGCTGCAGAATGAGAAACTGTACCTC
TACTACCTCCAGAATGGAAGGGACATGTACGTCGACCAAGAACTCGACATCAACAGACTGAGC
GACTACGATGTCGACCACATCGTGCCCCAGAGCTTTCTGAAAGACGACTCCATCGATAACAAG
GTCCTCACAAGATCCGACAAGAACAGAGGCAAGAGCGACAACGTCCCCTCCGAAGAGGTGGTG
AAAAAGATGAAGAACTACTGGAGGCAGCTGCTGAACGCCAAACTCATCACCCAGAGGAAGTTC
GATAATCTGACCAAAGCCGAAAGAGGAGGACTGTCCGAACTGGACAAAGCCGGCTTTATCAAG
AGGCAGCTGGTGGAAACCAGACAGATCACCAAACATGTCGCCCAAATTCTGGACTCTAGAATG
AACACCAAGTACGACGAAAATGACAAGCTGATTAGAGAAGTGAAGGTCATCACCCTCAAGAGC
AAGCTGGTCTCCGATTTTAGAAAGGATTTCCAATTCTACAAGGTCAGAGAGATCAATAATTAC
CACCATGCCCACGATGCCTATCTGAACGCCGTGGTGGGAACAGCCCTCATCAAGAAGTACCCT
AAGCTGGAAAGCGAGTTCGTGTATGGAGATTATAAAGTCTACGATGTGAGGAAGATGATTGCC
AAGTCCGAGCAAGAGATCGGCAAGGCCACCGCTAAATACTTCTTTTATTCCAACATCATGAAC
TTCTTTAAAACCGAGATCACACTCGCTAATGGCGAGATTAGGAAGAGACCTCTGATCGAGACA
AACGGCGAGACCGGCGAGATCGTCTGGGACAAGGGCAGAGATTTCGCCACCGTGAGAAAGGTG
CTCTCCATGCCTCAAGTGAACATCGTGAAAAAGACCGAGGTGCAGACCGGCGGCTTCTCCAAG
GAGTCCATTCTGCCCAAAAGGAACTCCGACAAGCTCATCGCTAGAAAGAAGGATTGGGATCCT
AAGAAATACGGCGGATTTGACTCCCCTACAGTCGCTTACAGCGTGCTCGTGGTGGCCAAGGTC
GAGAAGGGCAAGTCCAAGAAGCTGAAGTCCGTGAAGGAGCTGCTGGGAATCACAATCATGGAG
AGGTCCTCCTTCGAGAAGAACCCCATCGATTTTCTGGAGGCCAAGGGCTACAAAGAGGTGAAG
AAAGATCTGATCATTAAGCTGCCCAAATATTCCCTCTTCGAGCTGGAGAACGGAAGAAAAAGG
ATGCTGGCCTCCGCTGGCGAACTGCAGAAGGGAAACGAGCTCGCTCTCCCCAGCAAGTACGTC
AACTTCCTCTACCTCGCCAGCCACTACGAGAAACTGAAGGGATCCCCCGAGGACAATGAGCAG
AAGCAGCTCTTCGTGGAGCAGCACAAGCATTACCTCGATGAGATCATCGAGCAGATCTCCGAA
TTCAGCAAGAGGGTCATTCTGGCTGACGCCAACCTCGATAAGGTCCTCAGCGCTTACAACAAG
CACAGAGATAAGCCCATTAGGGAGCAAGCCGAAAATATCATCCATCTGTTTACACTGACAAAT
CTGGGCGCCCCCGCCGCTTTTAAGTACTTCGATACCACCATCGATAGAAAGAGGTACACCTCC
ACAAAAGAGGTGCTGGATGCTACCCTCATCCATCAGTCCATTACCGGACTCTACGAGACCAGA
ATTGATCTCTCCCAGCTGGGAGGAGATTCCGGCGGCAGCGGAGGAAGCGGCGGATCCACCAAT
CTGTCCGACATTATCGAGAAGGAGACCGGAAAACAACTCGTGATCCAAGAGTCCATCCTCATG
CTGCCCGAGGAAGTCGAGGAAGTGATCGGAAATAAGCCCGAGAGCGATATTCTGGTGCATACC
GCTTACGACGAGAGCACCGACGAAAATGTCATGCTGCTGACCTCCGATGCTCCCGAGTACAAA
CCTTGGGCTCTCGTCATTCAAGACAGCAACGGAGAGAACAAGATTAAGATGCTCAGCGGCGGA
AGCGGAGGCAGCGGCGGCTCCACAAATCTGTCCGATATCATCGAAAAGGAGACCGGCAAGCAA
CTGGTGATCCAAGAGAGCATTCTGATGCTCCCCGAAGAGGTGGAAGAGGTGATCGGCAATAAA
CCCGAGAGCGACATTCTGGTGCACACAGCCTACGATGAGTCCACCGATGAGAACGTGATGCTG
CTGACCAGCGATGCCCCCGAATATAAGCCTTGGGCTCTGGTGATTCAAGACTCCAATGGAGAG

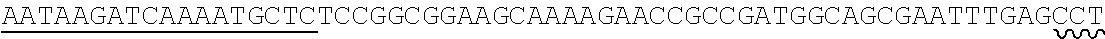

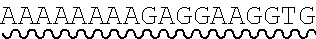

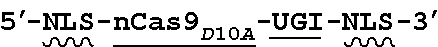

RNA scaffold expression cassette (S. pyogenes), containing a 20-
nucleotide programmable sequence, a CRISPR RNA motif, and an MS2
operator motif (SEQ ID NO: 70):
N₂₀GTTTTAGAGCTAGAAATAGCAAGTTAAAATAAGGCTAGTCCGTTATCAACTTGAAAAAGTG
GCACCGAGTCGGTGCGCGCACATGAGGATCACCCATGTGCTTTTTTTG
(N₂₀: programmable sequence; Underlined: CRISPR RNA motif;
Bold: MS2 motif; Italic: terminator)
The above RNA scaffold containing one MS2 loop (1xMS2).
Shown below is an example sequence encoding an RNA scaffold
containing two MS2 loops (2xMS2), where MS2 scaffolds are
underlined:
(SEQ ID NO: 71)
GTTTTAGAGCTAGAAATAGCAAGTTAAAATAAGGCTAGTCCGTTATCAACTTGAAAAAGTGGC
ACCGAGTCGGTGCgggagcACATGAGGATCACCCATGTgccacgagcgACATGAGGATCACCC
ATGTcgctcgtgttcccTTTTTTTCTCCGCT
Effector AID-MCP fusion protein sequence (SEQ ID NO: 72):
MDSLLMNRRKFLYQFKNVRWAKGRRETYLCYVVKRRDSATSFSLDFGYLRNKNGCHVELLFLR
YISDWDLDPGRCYRVTWFTSWSPCYDCARHVADFLRGNPNLSLRIFTARLYFCEDRKAEPEGL
RRLHRAGVQIAIMTFKDYFYCWNTFVENHERTFKAWEGLHENSVRLSRQLRRILLPLYEVDDL

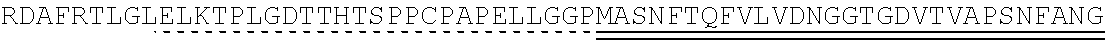

IAEWISSNSRSQAYKVTCSVRQSSAQNRKYTIKVEVPKGAWRSYLNMELTIPIFATNSDCELI
VKAMQGLLKDGNPIPSAIAANSGIY

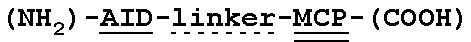

Codon optimized cDNA encoding effector human AID-MCP fusion
(SEQ ID NO: 73):

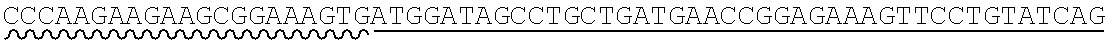

TTTAAGAATGTGCGCTGGGCAAAGGGCAGGCGCGAGACCTACCTGTGCTATGTGGTGAAGCGG
AGAGATTCCGCCACATCCTTCTCTCTGGACTTTGGCTACCTGCGGAACAAGAATGGCTGCCAC
GTGGAGCTGCTGTTCCTGAGATACATCTCTGACTGGGATCTGGACCCAGGCAGGTGTTATCGC
GTGACCTGGTTCACAAGCTGGTCCCCCTGCTACGATTGTGCAAGGCACGTGGCAGACTTTCTG
AGGGGAAACCCAAATCTGTCCCTGCGGATCTTCACCGCCAGACTGTATTTTTGCGAGGATAGG
AAGGCAGAGCCAGAGGGACTGAGGCGCCTGCACAGGGCCGGCGTGCAGATCGCCATCATGACC
TTCAAGGACTACTTTTATTGTTGGAACACCTTCGTGGAGAATCACGAGCGGACCTTCAAGGCC
TGGGAGGGACTGCACGAGAACTCCGTGCGGCTGTCTAGACAGCTGCGGAGAATCCTGCTGCCT

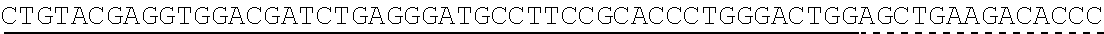

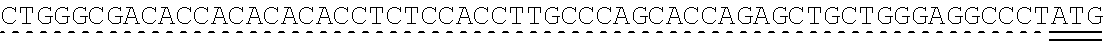

GCCAGCAACTTCACACAGTTTGTGCTGGTGGATAATGGAGGAACCGGCGACGTGACAGTGGCA
CCATCTAACTTTGCCAATGGCATCGCCGAGTGGATCAGCTCCAACTCTCGGAGCCAGGCCTAT
AAGGTGACCTGTAGCGTGCGGCAGTCTAGCGCCCAGAATAGAAAGTATACAATCAAGGTGGAG
GTGCCTAAGGGCGCCTGGAGATCCTACCTGAACATGGAGCTGACCATCCCAATCTTTGCCACA
AATTCTGATTGCGAGCTGATCGTGAAGGCCATGCAGGGCCTGCTGAAGGACGGCAACCCTATC
CCAAGCGCCATCGCCGCCAATAGCGGAATCTAC

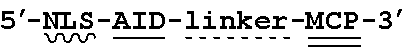

Codon optimized cDNA encoding effector rat APOBEC1-MCP fusion
sequence 1 (SEQ ID NO: 74):

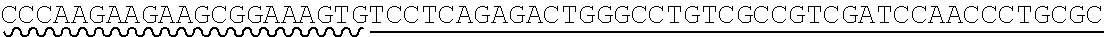

CGCCGGATTGAACCTCACGAGTTTGAAGTGTTCTTTGACCCCCGGGAGCTGAGAAAGGAGACA
TGCCTGCTGTACGAGATCAACTGGGGAGGCAGGCACTCCATCTGGAGGCACACCTCTCAGAAC
ACAAATAAGCACGTGGAGGTGAACTTCATCGAGAAGTTTACCACAGAGCGGTACTTCTGCCCC
AATACCAGATGTAGCATCACATGGTTTCTGAGCTGGTCCCCTTGCGGAGAGTGTAGCAGGGCC
ATCACCGAGTTCCTGTCCAGATATCCACACGTGACACTGTTTATCTACATCGCCAGGCTGTAT
CACCACGCAGACCCAAGGAATAGGCAGGGCCTGCGCGATCTGATCAGCTCCGGCGTGACCATC
CAGATCATGACAGAGCAGGAGTCCGGCTACTGCTGGCGGAACTTCGTGAATTATTCTCCTAGC
AACGAGGCCCACTGGCCTAGGTACCCACACCTGTGGGTGCGCCTGTACGTGCTGGAGCTGTAT
TGCATCATCCTGGGCCTGCCCCCTTGTCTGAATATCCTGCGGAGAAAGCAGCCCCAGCTGACC
TTCTTTACAATCGCCCTGCAGTCTTGTCACTATCAGAGGCTGCCACCCCACATCCTGTGGGCC

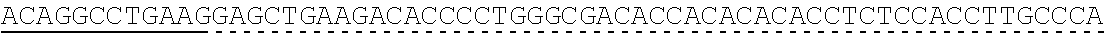

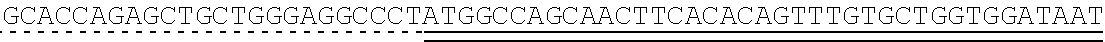

GGAGGAACCGGCGACGTGACAGTGGCACCATCTAACTTTGCCAATGGCATCGCCGAGTGGATC
AGCTCCAACTCTCGGAGCCAGGCCTATAAGGTGACCTGTAGCGTGCGGCAGTCTAGCGCCCAG
AATAGAAAGTATACAATCAAGGTGGAGGTGCCTAAGGGCGCCTGGAGATCCTACCTGAACATG
GAGCTGACCATCCCAATCTTTGCCACAAATTCTGATTGCGAGCTGATCGTGAAGGCCATGCAG
GGCCTGCTGAAGGACGGCAACCCTATCCCAAGCGCCATCGCCGCCAATAGCGGAATCTAC

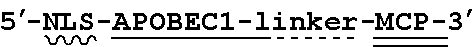

Codon optimized cDNA encoding effector rat APOBEC1-MCP fusion
sequence 2 (SEQ ID NO: 75):

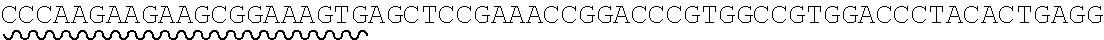

AGAAGGATCGAGCCCCACGAGTTTGAGGTGTTCTTCGACCCCAGAGAACTGAGGAAGGAGACA
TGTCTGCTGTATGAGATCAACTGGGGCGGAAGACACTCCATCTGGAGGCACACAAGCCAGAAC
ACCAACAAGCACGTCGAGGTGAACTTCATCGAGAAGTTCACCACCGAGAGGTACTTCTGCCCC
AACACAAGATGCTCCATCACATGGTTTCTGAGCTGGAGCCCTTGCGGCGAATGCTCCAGAGCC
ATCACCGAGTTTCTGTCTAGATACCCCCACGTGACACTGTTTATCTACATCGCTAGACTGTAC
CACCATGCCGATCCCAGAAACAGACAAGGACTGAGGGATCTGATCTCCAGCGGCGTGACCATC
CAGATCATGACCGAGCAAGAGTCCGGCTACTGCTGGAGGAACTTCGTGAACTACTCCCCTAGC
AACGAGGCCCACTGGCCCAGATACCCTCATCTGTGGGTGAGACTGTACGTGCTCGAGCTGTAC
TGTATCATTCTGGGACTGCCTCCTTGTCTGAACATTCTGAGAAGGAAGCAGCCCCAGCTGACC
TTCTTCACCATCGCTCTGCAGAGCTGCCACTACCAGAGGCTGCCTCCCCACATTCTGTGGGCC

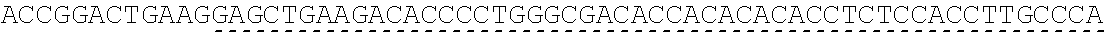

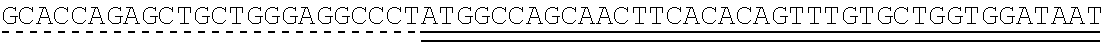

GGAGGAACCGGCGACGTGACAGTGGCACCATCTAACTTTGCCAATGGCATCGCCGAGTGGATC
AGCTCCAACTCTCGGAGCCAGGCCTATAAGGTGACCTGTAGCGTGCGGCAGTCTAGCGCCCAG
AATAGAAAGTATACAATCAAGGTGGAGGTGCCTAAGGGCGCCTGGAGATCCTACCTGAACATG
GAGCTGACCATCCCAATCTTTGCCACAAATTCTGATTGCGAGCTGATCGTGAAGGCCATGCAG
GGCCTGCTGAAGGACGGCAACCCTATCCCAAGCGCCATCGCCGCCAATAGCGGAATCTAC

Like the Cas protein described above, the non-nuclease effector can also be obtained as a recombinant polypeptide. Techniques for making recombinant polypeptides are known in the art. See e.g., Creighton, “Proteins: Structures and Molecular Principles,” W.H. Freeman & Co., NY, 1983); Ausubel et al., Current Protocols in Molecular Biology, John Wiley & Sons, 2003; and Sambrook et al., Molecular Cloning, A Laboratory Manual,” Cold Spring Harbor Press, Cold Spring Harbor, N.Y., 2001).

As described herein, by mutating Ser38 to Ala in AID one can reduce the recruitment of AID to off-target sites. Listed below are the DNA and protein sequences of both wild type AID as well as AID_S38A (phosphorylation null, pnAID):

MAID protein (Ser38 in bold and underlined, SEQ
ID NO: 76):
MDSLLMNRRKFLYQFKNVRWAKGRRETYLCYVVKRRDSATSFSLDEGYL
RNKNGCHVELLFLRYISDWDLDPGRCYRVTWFTSWSPCYDCARHVADFL
RGNPNLSLRIFTARLYFCEDRKAEPEGLRRLHRAGVQIAIMTEKDYFYC
WNTFVENHERTFKAWEGLHENSVRLSRQLRRILLPLYEVDDLRDAFRTL
GL
MAID cDNA (Ser38 codon in bold and underlined,
SEQ ID NO: 77):
ATGGACAGCCTCTTGATGAACCGGAGGAAGTTTCTTTACCAATTCAAAA
ATGTCCGCTGGGCTAAGGGTCGGCGTGAGACCTACCTGTGCTACGTAGT
GAAGAGGCGTGACAGTGCTACATCCTTTTCACTGGACTTTGGTTATCTT
CGCAATAAGAACGGCTGCCACGTGGAATTGCTCTTCCTCCGCTACATCT
CGGACTGGGACCTAGACCCTGGCCGCTGCTACCGCGTCACCTGGTTCAC
CTCCTGGAGCCCCTGCTACGACTGTGCCCGACATGTGGCCGACTTTCTG
CGAGGGAACCCCAACCTCAGTCTGAGGATCTTCACCGCGCGCCTCTACT
TCTGTGAGGACCGCAAGGCTGAGCCCGAGGGGCTGCGGCGGCTGCACCG
CGCCGGGGTGCAAATAGCCATCATGACCTTCAAAGATTATTTTTACTGC
TGGAATACTTTTGTAGAAAACCATGAAAGAACTTTCAAAGCCTGGGAAG
GGCTGCATGAAAATTCAGTTCGTCTCTCCAGACAGCTTCGGCGCATCCT
TTTGCCCCTGTATGAGGTTGATGACTTACGAGACGCATTTCGTACTTTG
GGACTT
Codon optimized MAID cDNA (Ser38 codon in bold
and underlined, SEQ ID NO: 78):
ATGGATAGCCTGCTGATGAACCGGAGAAAGTTCCTGTATCAGTTTAAGA
ATGTGCGCTGGGCAAAGGGCAGGCGCGAGACCTACCTGTGCTATGTGGT
GAAGCGGAGAGATTCCGCCACATCCTTCTCTCTGGACTTTGGCTACCTG
CGGAACAAGAATGGCTGCCACGTGGAGCTGCTGTTCCTGAGATACATCT
CTGACTGGGATCTGGACCCAGGCAGGTGTTATCGCGTGACCTGGTTCAC
AAGCTGGTCCCCCTGCTACGATTGTGCAAGGCACGTGGCAGACTTTCTG
AGGGGAAACCCAAATCTGTCCCTGCGGATCTTCACCGCCAGACTGTATT
TTTGCGAGGATAGGAAGGCAGAGCCAGAGGGACTGAGGCGCCTGCACAG
GGCCGGCGTGCAGATCGCCATCATGACCTTCAAGGACTACTTTTATTGT
TGGAACACCTTCGTGGAGAATCACGAGCGGACCTTCAAGGCCTGGGAGG
GACTGCACGAGAACTCCGTGCGGCTGTCTAGACAGCTGCGGAGAATCCT
GCTGCCTCTGTACGAGGTGGACGATCTGAGGGATGCCTTCCGCACCCTG
GGACTG
AID_S38A protein (S38A mutation in bold and
underlined, SEQ ID NO: 79)
MDSLLMNRRKFLYQFKNVRWAKGRRETYLCYVVKRRDAATSFSLDEGYL
RNKNGCHVELLFLRYISDWDLDPGRCYRVTWFTSWSPCYDCARHVADFL
RGNPNLSLRIFTARLYFCEDRKAEPEGLRRLHRAGVQIAIMTEKDYFYC
WNTFVENHERTFKAWEGLHENSVRLSRQLRRILLPLYEVDDLRDAFRTL
GL
AID_S38A cDNA (S38A mutation in bold and
underlined, SEQ ID NO: 80)
ATGGACAGCCTCTTGATGAACCGGAGGAAGTTTCTTTACCAATTCAAAA
ATGTCCGCTGGGCTAAGGGTCGGCGTGAGACCTACCTGTGCTACGTAGT
GAAGAGGCGTGACGCCGCTACATCCTTTTCACTGGACTTTGGTTATCTT
CGCAATAAGAACGGCTGCCACGTGGAATTGCTCTTCCTCCGCTACATCT
CGGACTGGGACCTAGACCCTGGCCGCTGCTACCGCGTCACCTGGTTCAC
CTCCTGGAGCCCCTGCTACGACTGTGCCCGACATGTGGCCGACTTTCTG
CGAGGGAACCCCAACCTCAGTCTGAGGATCTTCACCGCGCGCCTCTACT
TCTGTGAGGACCGCAAGGCTGAGCCCGAGGGGCTGCGGCGGCTGCACCG
CGCCGGGGTGCAAATAGCCATCATGACCTTCAAAGATTATTTTTACTGC
TGGAATACTTTTGTAGAAAACCATGAAAGAACTTTCAAAGCCTGGGAAG
GGCTGCATGAAAATTCAGTTCGTCTCTCCAGACAGCTTCGGCGCATCCT
TTTGCCCCTGTATGAGGTTGATGACTTACGAGACGCATTTCGTACTTTG
GGACTT
Codon optimized AID_S38A cDNA (S38A mutation in
bold and underlined, SEQ ID NO: 81)
ATGGATAGCCTGCTGATGAACCGGAGAAAGTTCCTGTATCAGTTTAAGA
ATGTGCGCTGGGCAAAGGGCAGGCGCGAGACCTACCTGTGCTATGTGGT
GAAGCGGAGAGATGCCGCCACATCCTTCTCTCTGGACTTTGGCTACCTG
CGGAACAAGAATGGCTGCCACGTGGAGCTGCTGTTCCTGAGATACATCT
CTGACTGGGATCTGGACCCAGGCAGGTGTTATCGCGTGACCTGGTTCAC
AAGCTGGTCCCCCTGCTACGATTGTGCAAGGCACGTGGCAGACTTTCTG
AGGGGAAACCCAAATCTGTCCCTGCGGATCTTCACCGCCAGACTGTATT
TTTGCGAGGATAGGAAGGCAGAGCCAGAGGGACTGAGGCGCCTGCACAG
GGCCGGCGTGCAGATCGCCATCATGACCTTCAAGGACTACTTTTATTGT
TGGAACACCTTCGTGGAGAATCACGAGCGGACCTTCAAGGCCTGGGAGG
GACTGCACGAGAACTCCGTGCGGCTGTCTAGACAGCTGCGGAGAATCCT
GCTGCCTCTGTACGAGGTGGACGATCTGAGGGATGCCTTCCGCACCCTG
GGACTG

Exemplary Sequences

Shown below are a number of exemplary RNA sequence of gRNA constructs used in this study. Each contains, from the 5′ end to the 3′ end, a customizable target, a gRNA scaffold, and one or two copies of a MS2 aptamer.

Sequence of gRNA_MS2 construct (SEQ ID NO: 82):
NNNNNNNNNNNNNNNNNNNGUUUUAGAGCUAGAAAUAGCAAGUUAAA
AUAAGGCUAGUCCGUUAUCAACUUGAAAAAGUGGCACCGAGUCGGUG

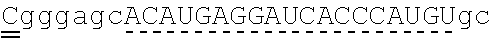

Sequence of gRNA_2xMS2 construct (SEQ ID NO: 83):
NNNNNNNNNNNNNNNNNNNGUUUUAGAGCUAGAAAUAGCAAGUUAAA
AUAAGGCUAGUCCGUUAUCAACUUGAAAAAGUGGCACCGAGUCGGUG

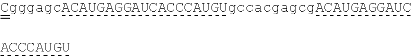

Key: Customizable target-gRNA scaffold-

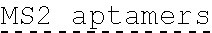

The above three components of the platform/system disclosed herein can be expressed using one, two or three expression vectors. The system can be programmed to target virtually any DNA or RNA sequence. In addition to the second generation CRC base editors described above, similar second generation CRC base editors could be generated by varying the modular components of the system, including any suitable Cas orthologs, deaminase orthologs, and other DNA modification enzymes.

### Expression System

To use the platform described above, it may be desirable to express one or more of the protein and RNA components from nucleic acids that encode them. This can be performed in a variety of ways. For example, the nucleic acids encoding the RNA scaffold or proteins can be cloned into one or more intermediate vectors for introducing into prokaryotic or eukaryotic cells for replication and/or transcription. Intermediate vectors are typically prokaryotic vectors, e.g., plasmids, or shuttle vectors, or insect vectors, for storage or manipulation of the nucleic acid encoding the RNA scaffold or protein for production of the RNA scaffold or protein. The nucleic acids can also be cloned into one or more expression vectors, for administration to a plant cell, animal cell, preferably a mammalian cell or a human cell, fungal cell, bacterial cell, or protozoan cell. Accordingly, the present invention provides nucleic acids that encode any of the RNA scaffold or proteins mentioned above. Preferably, the nucleic acids are isolated and/or purified.

The present invention also provides recombinant constructs or vectors having sequences encoding one or more of the RNA scaffold or proteins described above. Examples of the constructs include a vector, such as a plasmid or viral vector, into which a nucleic acid sequence of the invention has been inserted, in a forward or reverse orientation. In a preferred embodiment, the construct further includes regulatory sequences, including a promoter, operably linked to the sequence. Large numbers of suitable vectors and promoters are known to those of skill in the art, and are commercially available. Appropriate cloning and expression vectors for use with prokaryotic and eukaryotic hosts are also described in e.g., Sambrook et al. (2001, Molecular Cloning: A Laboratory Manual, Cold Spring Harbor Press).

A vector refers to a nucleic acid molecule capable of transporting another nucleic acid to which it has been linked. The vector can be capable of autonomous replication or integration into a host DNA. Examples of the vector include a plasmid, cosmid, or viral vector. The vector of this invention includes a nucleic acid in a form suitable for expression of the nucleic acid in a host cell. Preferably, the vector includes one or more regulatory sequences operatively linked to the nucleic acid sequence to be expressed. A “regulatory sequence” includes promoters, enhancers, and other expression control elements (e.g., polyadenylation signals). Regulatory sequences include those that direct constitutive expression of a nucleotide sequence, as well as inducible regulatory sequences. The design of the expression vector can depend on such factors as the choice of the host cell to be transformed, transfected, or transduced, the level of expression of RNAs or proteins desired, and the like.

Examples of expression vectors include chromosomal, non-chromosomal and synthetic DNA sequences, bacterial plasmids, phage DNA, baculovirus, yeast plasmids, vectors derived from combinations of plasmids and phage DNA, viral DNA such as vaccinia, adenovirus, fowl pox virus, and pseudorabies. However, any other vector may be used provided it is replicable and viable in the host. The appropriate nucleic acid sequence may be inserted into the vector by a variety of procedures. In general, a nucleic acid sequence encoding one of the RNAs or proteins described above can be inserted into an appropriate restriction endonuclease site(s) by procedures known in the art. Such procedures and related sub-cloning procedures are within the scope of those skilled in the art.

The vector may include appropriate sequences for amplifying expression. In addition, the expression vector preferably contains one or more selectable marker genes to provide a phenotypic trait for selection of transformed host cells such as dihydrofolate reductase or neomycin resistance for eukaryotic cell cultures, or such as tetracycline or ampicillin resistance in E. coli.

The vectors for expressing the RNAs can include RNA Pol III promoters to drive expression of the RNAs, e.g., the HI, U6 or 7SK promoters. These human promoters allow for expression of RNAs in mammalian cells following plasmid transfection. Alternatively, a T7 promoter may be used, e.g., for in vitro transcription, and the RNA can be transcribed in vitro and purified.

The vector containing the appropriate nucleic acid sequences as described above, as well as an appropriate promoter or control sequence, can be employed to transform, transfect, or infect an appropriate host to permit the host to express the RNAs or proteins described above. Examples of suitable expression hosts include bacterial cells (e.g., E. coli, Streptomyces, Salmonella typhimurium), fungal cells (yeast), insect cells (e.g., Drosophila and Spodoptera frugiperda (Sf9)), animal cells (e.g., CHO, COS, and HEK 293), adenoviruses, and plant cells. The selection of an appropriate host is within the scope of those skilled in the art. In some embodiments, the present invention provides methods for producing the above mentioned RNAs or proteins by transforming, transfecting, or infecting a host cell with an expression vector having a nucleotide sequence that encodes one of the RNAs, or polypeptides, or proteins. The host cells are then cultured under a suitable condition, which allows for the expression of the RNAs or proteins.

Any of the procedures known in the art for introducing foreign nucleotide sequences into host cells may be used. Examples include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell.

### Methods

Another aspect of the present invention encompasses a method for modifying a target DNA sequence (e.g., a chromosomal sequence) or target RNA sequence in a cell, embryo, human or non-human animals. The method comprises introducing into the cell or embryo the above-described (i) a sequence-targeting protein, or a polynucleotide encoding the same, (ii) an RNA scaffold, or a DNA polynucleotide encoding the same, and (iii) a non-nuclease effector fusion protein, or a polynucleotide encoding the same. The RNA scaffold guides the sequence-targeting protein and the fusion protein to a target polynucleotide at a target site and the effector domain of the fusion protein modifies the sequence. As disclosed herein, the sequence-targeting protein, such as a Cas9 protein, is modified such that the endonuclease activity is eliminated.

In certain embodiments, the effector protein functions as a monomer. In that case, the system of this invention can be targeted to a single site, either upstream (left) or downstream (right) of the target site as shown in, e.g., WO2018129129 FIG. 1C. In other embodiments, the effector protein requires dimerization for proper catalytic function. To that end, the system can be multiplexed to target sequences upstream and downstream of the target site simultaneously, therefore allowing the effector proteins to dimerize (as shown in, e.g., WO2018129129 FIG. 1D, left). Alternatively, recruitment of effector protein to a single site may be sufficient to increase its affinity for neighboring effector proteins, promoting dimerization (as shown in, e.g., WO2018129129 FIG. 1D, right). In yet some other embodiments, a tetramer effector enzyme can be recruited and positioned at the target site as shown in, e.g., WO2018129129 FIG. 1E. This can be achieved by dual or single targeting (as shown in, e.g., WO2018129129 FIG. 1E, left and right). The system disclosed in this invention can be used to edit RNA targets too (e.g., retrovirus inactivation). In that case, if the effector protein requires assembly of a functional oligomer, single targeting to an RNA molecule could promote oligomerization as shown in, e.g., WO2018129129.

The target polynucleotide has no sequence limitation except that the sequence is immediately followed (downstream or 3′) by a PAM sequence. Examples of PAM include, but are not limited to, NGG, NGGNG, and NNAGAAW (wherein N is defined as any nucleotide and W is defined as either A or T). Other examples of PAM sequences are given above, and the skilled person will be able to identify further PAM sequences for use with a given CRISPR protein. The target site can be in the coding region of a gene, in an intron of a gene, in a control region between genes, etc. The gene can be a protein-coding gene or an RNA coding gene.

The target polynucleotide can be any polynucleotide endogenous or exogenous to the cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide).

The protein components of this system of this invention can be introduced into the cell or embryo as an isolated protein. Alternatively, the components can be introduced via nucleic acids encoding such components, such DNA or RNA (e.g., in vitro transcribed RNA). In one embodiment, each protein can comprise at least one cell-penetrating domain, which facilitates cellular uptake of the protein. In other embodiments, mRNA molecules or DNA molecules encoding the protein or proteins can be introduced into the cell or embryo. In general, a DNA sequence encoding the protein is operably linked to a promoter sequence that will function in the cell or embryo of interest. The DNA sequence can be linear, or the DNA sequence can be part of a vector. In still other embodiments, the protein can be introduced into the cell or embryo as an RNA-protein complex comprising the protein and the RNA scaffold described above.

In alternate embodiments, DNA encoding the protein(s) can further comprise a sequence or sequences encoding components of the RNA scaffold. In general, the DNA sequence encoding the protein and the RNA scaffold is operably linked to appropriate promoter control sequences that allow the expression of the protein and the RNA scaffold, respectively, in the cell or embryo. The DNA sequence encoding the protein and the RNA scaffold can further comprise additional expression control, regulatory, and/or processing sequence(s). The DNA sequence encoding the protein and the guiding RNA can be linear or can be part of a vector.

In embodiments in which the RNA is introduced into the cell via a DNA molecule encoding the RNA, the RNA coding sequence can be operably linked to promoter control sequence for expression of the guiding RNA in the eukaryotic cell. For example, the RNA coding sequence can be operably linked to a promoter sequence that is recognized by RNA polymerase III (Pol III). Examples of suitable Pol III promoters include, but are not limited to, mammalian U6 or H1 promoters. In exemplary embodiments, the RNA coding sequence is linked to a mouse or human U6 promoter. In other exemplary embodiments, the RNA coding sequence is linked to a mouse or human H1 promoter.

The DNA molecule encoding the protein and/or RNA can be linear or circular. In some embodiments, the DNA sequence can be part of a vector, such as a multi-cistronic vector. Suitable vectors include plasmid vectors, phagemids, cosmids, artificial/mini-chromosomes, transposons, and viral vectors. In an exemplary embodiment, the DNA encoding the protein and/or RNA is present in a plasmid vector. Non-limiting examples of suitable plasmid vectors include pUC, pBR322, pET, pBluescript, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like.

The protein components of this system of this invention (or nucleic acid(s) encoding them) and the RNA components (or DNAs encoding them) can be introduced into a cell or embryo by a variety of means. Typically, the embryo is a fertilized one-cell stage embryo of the species of interest. In some embodiments, the cell or embryo is transfected. Suitable transfection methods include calcium phosphate-mediated transfection, nucleofection (or electroporation), cationic polymer transfection (e.g., DEAE-dextran or polyethylenimine), viral transduction, virosome transfection, virion transfection, liposome transfection, cationic liposome transfection, immunoliposome transfection, nonliposomal lipid transfection, dendrimer transfection, heat shock transfection, magnetofection, lipofection, gene gun delivery, impalefection, sonoporation, optical transfection, gold nanoparticle-mediated transfection, and proprietary agent-enhanced uptake of nucleic acids. Transfection methods are well known in the art (see, e.g., “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001). In other embodiments, the molecules are introduced into the cell or embryo by microinjection. For example, the molecules can be injected into the pronuclei of one-cell embryos.

The protein components of this system of this invention (or nucleic acid(s) encoding them) and the RNA components (or DNAs encoding them) can be introduced into a cell or embryo simultaneously or sequentially. The ratio of the protein (or its encoding nucleic acid) to the RNA (or DNAs encoding the RNA), generally will be approximately stoichiometric such that they can form an RNA-protein complex. Similarly, the ratio of two different proteins (or encoding nucleic acids) will be approximately stoichiometric. In one embodiment, the protein components and the RNA components (or the DNA sequences encoding them) are delivered together within the same nucleic acid or vector.

The method further comprises maintaining the cell or embryo under appropriate conditions such that the guide RNA guides the effector protein to the targeted site in the target sequence, and the effector domain modifies the target sequence.

In general, the cell can be maintained under conditions appropriate for cell growth and/or maintenance. Suitable cell culture conditions are well known in the art and are described, for example, in Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001), Santiago et al. (2008) PNAS 105:5809-5814; Moehle et al. (2007) PNAS 104:3055-3060; Urnov et al. (2005) Nature 435:646-651; and Lombardo et al. (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

An embryo can be cultured in vitro (e.g., in cell culture). Typically, the embryo is cultured at an appropriate temperature and in appropriate media with the necessary O₂/CO₂ ratio to allow the expression of the proteins and RNA scaffold, if necessary. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

Alternatively, an embryo may be cultured in vivo by transferring the embryo into a uterus of a female host. Generally speaking, the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and can result in a live birth of an animal derived from the embryo. Such an animal would comprise the modified chromosomal sequence in every cell of the body.

A variety of eukaryotic cells are suitable for use in the method. For example, the cell can be a human cell, a non-human mammalian cell, a non-mammalian vertebrate cell, an invertebrate cell, an insect cell, a plant cell, a yeast cell, or a single cell eukaryotic organism. A variety of embryos are suitable for use in the method. For example, the embryo can be a 1-cell, 2-cell, or 4-cell human or non-human mammalian embryo. Exemplary mammalian embryos, including one cell embryos, include without limit mouse, rat, hamster, rodent, rabbit, feline, canine, ovine, porcine, bovine, equine, and primate embryos. In still other embodiments, the cell can be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, unipotent stem cells and others. In exemplary embodiments, the cell is a mammalian cell, or the embryo is a mammalian embryo.

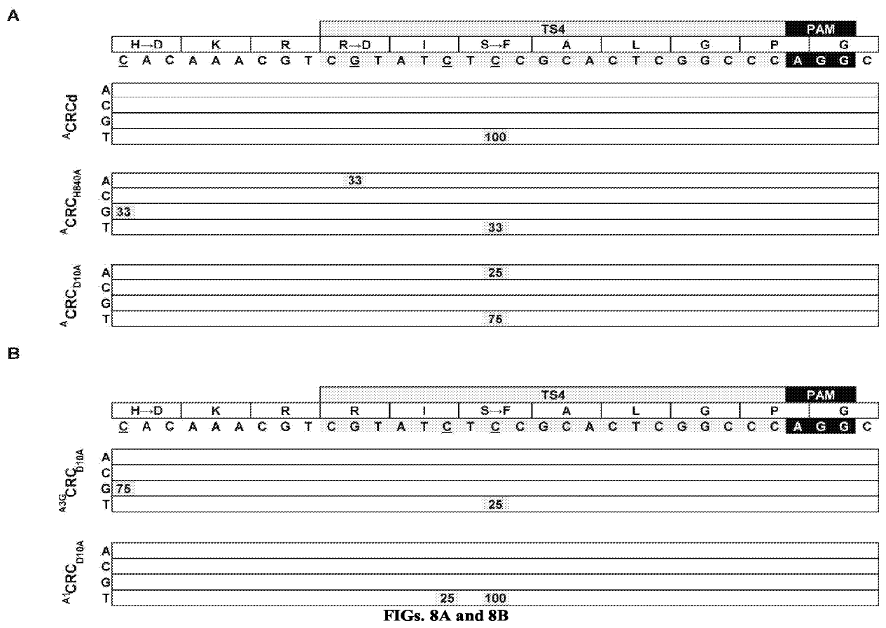

As shown in WO2018129129, a study was performed applying this Cis Double Nicking Technology to enhance conversion efficiency in a bacterial gene conversion model. Experimentally, nCas9 (nCas9_{D10A} or nCas9_{H840}) were programmed to target two neighboring positions on the same DNA strand. Double nicking the same strand with two gRNA does not induce double strand DNA breaks or activation of DSB repair pathways, therefore this is a safe approach. A schematic of the procedure is described in FIG. 8 of WO2018129129. To test this approach, the bacterial gene encoding for the RNA polymerase 13 subunit (rpoB) was targeted using gRNAs TS-2 and TS-3. This is a negative selection system, in which specific rpoB mutants can be selected using the antibiotic rifampicin since mutants are resistant to this drug (Rif^{R}). The results in prokaryotic cells suggest that targeting efficiency can be enhanced up to 100-fold.

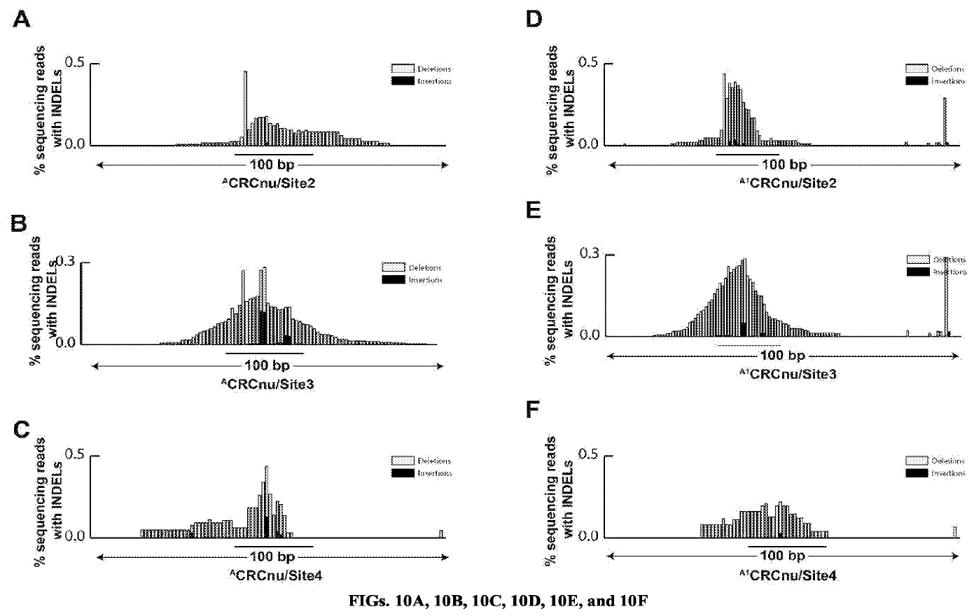

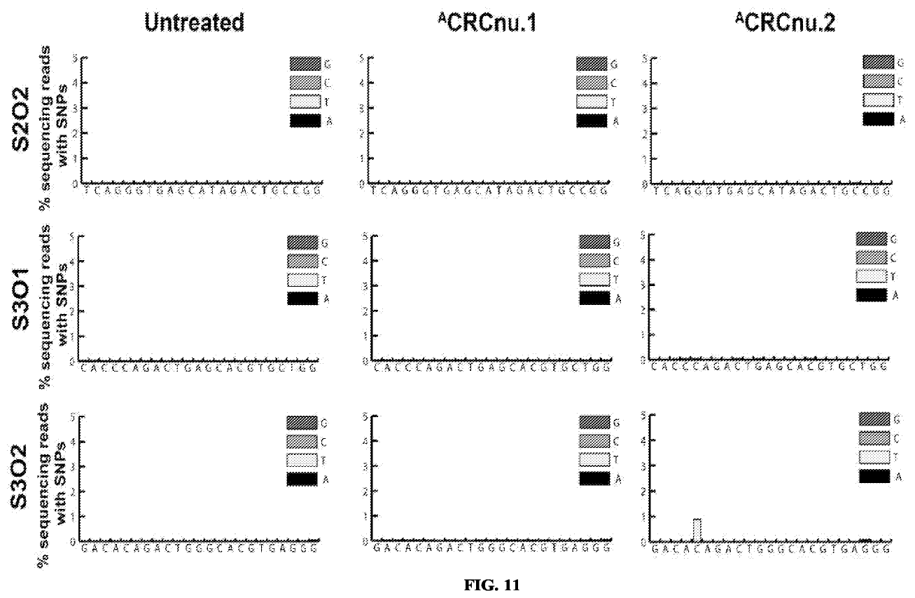

By harnessing CRC's modular design, this invention also provides a method that can recruit two effectors (either the same or different) to a target sequence, synergistically enhance the genetic conversion. These designs are exemplified in FIG. 10 of WO2018129129. For example, both gRNAs can be engineered to have the same recruiting RNA motif (e.g., MS2 scaffold), CRC effector fused to MCP protein can be recruited to both nicking sites. This allows one to recruit two identical effectors to the target sequence, increasing local concentration of the effectors or facilitating dimerization or multimerization required for effector functions.

Likewise, this invention also provides a method that can recruit or exclude a CRC effector from any of the nicking sites by selecting gRNAs with or without recruiting RNA motif, respectively. This allows to recruit one effector but exposing a single stranded DNA, facilitating effector function.

In another example, this invention provides a method that recruits two different functional effectors into the same target sequence. The two effectors work together synergistically to facilitate the genetic conversion. For example, to further increase targeting efficiency, one can program CRC recruitment of a deaminase (e.g., AID) to the nicking site closer to the target nucleotide, and a local DNA repair inhibitor to the second nicking site (e.g., UNG inhibitor, UGI). While the AID facilitates the conversion of, e.g., C to T at the target sequence, the UGI inhibits the endogenous repair pathway locally. These two effectors thus cooperate specifically at the target site to enhance conversion efficacy. To avoid crosstalk between the CRC recruitment site and the inhibitor recruitment site, orthogonal recruiting RNA motifs can be used for each of these modules (e.g., MS2-MCP recruits CRC effector AID fused to MCP and PP7-PCP recruits UGI fused to PCP).

In some embodiments, the hetero-recruitment configuration can also be applied if heterodimerization is required for proper effector activity. The hetero-recruitment configuration can also be applied to any gene conversion enzyme system requiring at least two components to function effectively. A non-exhaustive list of recruiting RNA scaffolds and their RNA binding protein partners is summarized in Table 2. Finally, if there are PAM sequence restrictions for cis double nicking, it is also possible to program Cas9 orthologs from species other than S. pyogenes, depending on what PAM sequences are available near the target sites. A non-exhaustive list of Cas9 orthologs from different species is summarized in Table 1.

A fundamental difference between BE and CRC is the mechanism by which the effector DNA modification enzyme is recruited to the target site. BE is mediated by a direct fusion between Cas9 and the effector, while CRC is mediated through an RNA aptamer on the 3′ of gRNA which in turn recruits its cognate aptamer ligand fusing to the effector. An appealing feature of the CRC system is the modular design: the functionality of DNA recognition and effector action reside in separate molecules, and the interaction of the two functional modules is coded by a gRNA molecule that can be easily reprogrammed. As such, the CRISPR protein module and the effector module can be individually engineered/optimized without interfering with each other, as well attested in this study. In addition, the CRC design could potentially make it easier for simultaneously targeting different sites with different types of effectors (multiplexing). For example, one may introduce an A to G effector (adenine deaminase) and a C to T effector (cytidine deaminase) into the same cell for targeting different sequences; or target one site for transcriptional activation (transient) and a second site for stop-codon knockout (permanently).

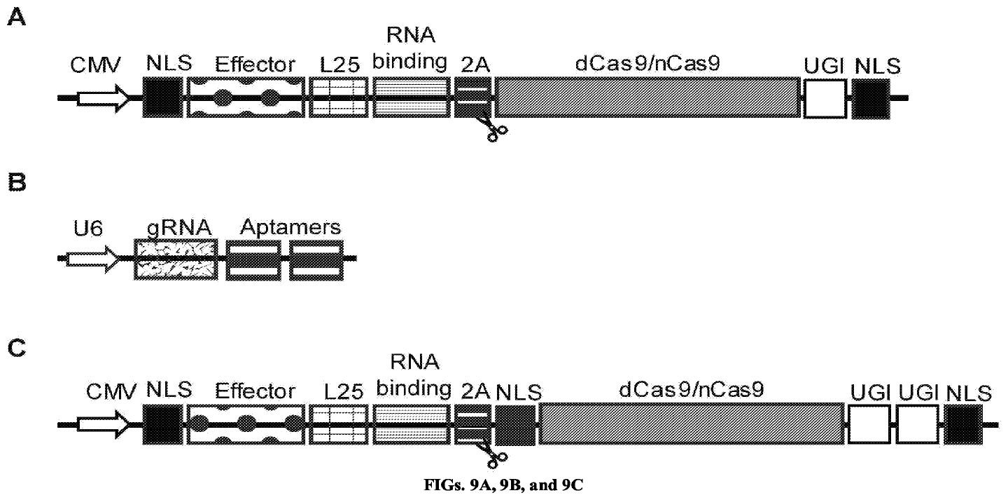

In the study presented in the examples below, two best CRC constructs, Gen2 CRC_AID (^{A}CRCnu.2) and Gen2_CRC_APOBEC1 (^{A1}CRCnu.2) were characterized. They both consist of a codon optimized Cas9_{D10A} nickase fused with 2× UGI, a gRNA with two copies of MS2 aptamer linked at the 3′ end, and a codon optimized MCP-cytidine deaminase fusion protein (FIGS. 9B and 9C). The cytidine deaminases of ^{A}CRCnu.2 and ^{A1}CRCnu.2 are human AID and rat APOBEC1, respectively. The effector module from both Gen2 CRC systems contain one nuclear localization signal and a flexible hinge linker separating the cytidine deaminase from the RNA-aptamer ligand.

For example, in the tested target sites, the base editing activities of the two CRC constructs, despite being respectively different, are above 10% and could reach over 50%, and the off-target activities are generally absent or low depending on the guide sequence used. These CRC constructs have reached the general benchmarks and can be further tested and optimized in therapeutic settings such as in cells derived from patients and in animal disease models. One can use them in at least three different therapeutic modes: (1) base conversion (including correction of a disease-causing mutation and introduce a second site suppressor mutation), (2) pre-mature stop codon knockout, and (3) exon skipping.

In this invention, inventors tested the therapeutic mode of base correction of loss-of-function mutation in a reporter GFP gene, as well as the mode of stop codon knockout using a wild type GFP transgene and the endogenous PDCD1 gene, with high efficiency. As the 3′ splice site in almost all genes contains an AG consensus sequence (46, 47), the therapeutic strategy of exon skipping is viable for some disease genes should an optimal PAM motif be available near the target splicing site (48). Thus, base editing platforms can provide powerful therapeutics for permanently correcting disease-causing mutations (e.g., beta thalassemia), permanently knocking out gene expression (e.g., CAR-T cell engineering), as well as permanently skipping the expression of disease-causing exons (e.g., Duchenne muscular dystrophy), in both ex vivo and in vivo therapeutic settings.

The center of CRC platform is based on the foundation that the nuclease deficient CRISPR complex can serve as a DNA or RNA sequence specific targeting module. This foundation is also the base of a number of different systems engineered for other different purposes based on this foundation, either through an RNA-based or protein-based recruitment. In addition to the BE base editing systems, Feng Zhang group (16) and Stanley Qi group (15) have used the gRNA component and RNA aptamers for recruiting transcriptional regulation effectors to re-program the transcriptional network. Bassik group has placed the recruiting RNA aptamer at the tetraloop and stem loop 2 of gRNA for recruitment of a mutant, hyperactive cytidine deaminase (CRISPR-X system) (20). Interestingly, when the RNA aptamer is placed in these positions instead of the 3′ end of gRNA as in CRC system, CRISPR-X exhibits a distinct activity profile with cytidine deamination activity spanning a wide range around and beyond the target protospacer sequence at lower efficiency (20, 21). In conjugation with hyperactive variants of deaminase (AID), this property of CRISPRx was utilized for generating permutation and protein evolution/engineering in cells and in vitro. The system is particularly useful for creating antibody diversity (21). It is expected that the systems utilizing the CRISPR DNA/RNA sequence recognition module will further expand for the purpose of re-writing a genome or re-programming cellular programs. Accordingly, the same strategy can be used with the CRC system described herein.

### Utilities and Applications

The systems and methods disclosed herein have a wide variety of utilities including modifying and editing (e.g., inactivating and activating) a target polynucleotide in a multitude of cell types. As such the systems and methods have a broad spectrum of applications in, e.g., research and therapy. For example, the systems and methods can be used for high throughput screening where multiple systems with different guide RNAs target multiple different loci to obtain and screen for multiple different phenotypic outcomes (e.g., better proliferation or lethal screens in cell lines). In another example, the systems and methods can be used in mutagenesis (similar to CRISPR tiling) or genes to create novel proteins.

Many devastating human diseases have one common cause: genetic alteration or mutation. The disease-causing mutations in patients either are acquired through inheritance from their parents or are caused by environmental factors. These diseases include, but are not limited to, the following categories. First, some genetic disorders are caused by germline mutations. One example is cystic fibrosis, which is caused by mutations at the CFTR gene inherited from parents. A second suppressor mutation in the mutant CFTR can partially restore the function of CFTR protein in somatic tissues. Other example genetic diseases caused by a point genetic mutation that can be corrected by the invention include Gaucher's disease, alpha trypsin deficiency disease, sickle cell anemia, to name a few. Second, some diseases, such as chronic viral infectious diseases, are caused by exogenous environmental factors and resulting genetic alterations. One example is AIDS, which is caused by insertion of the human HIV viral genome into the genome of infected T-cells. Third, some neurodegenerative diseases involve genetic alterations. One example is Huntington's diseases, which is caused by expansion of CAG tri-nucleotide in the huntingtin gene of affected patients. Other examples include lysosomal storage diseases, Epidermolysis Bullosa, and retinal degeneration. Finally, cancers are caused by various somatic mutations accumulated in cancer cells. Therefore, correcting the disease-causing genetic mutations, or functionally correcting the sequence, provides an appealing therapeutic opportunity to treat these diseases.

Somatic genetic editing is an appealing therapeutic strategy for many human diseases. To achieve successful therapeutic genetic editing, three critical factors are considered essential: (i) how to achieve sequence specific recognition (“sequence recognition module”); (ii) how to correct the underlying mutations (“correction module”); and (iii) how to link the “correction module” to “sequence recognition module” together to achieve sequence specific correction. There is a number of ways of achieving each individual task. However, none of the currently existing platforms or technologies could achieve optimal and practical somatic genetic editing. More specifically, current gene specific editing technologies are mostly based on nucleases induced DNA DSB and consequent DSB induced homologous recombination, the activity of which is low or absent in most somatic cells. Thus, those technologies are of limited use for therapeutic corrections of pathological genetic mutations in somatic tissues in most diseases.

In contrast, the system and method disclosed in this invention allow DNA-sequence directed editing of a gene or RNA transcript that does not rely on nuclease activity. The system and method do not generate DSB, or do not rely on the DSB-mediated homologous recombination. Moreover, this design of the system is modular, which allows extremely flexible and convenient way of targeting any desirable DNA or RNA sequences. In essence, this approach enables one to guide a DNA or RNA editing enzyme to virtually any DNA or RNA sequence in somatic cells, including stem cells. Through precise editing of the target DNA or RNA sequence, the enzyme can correct the mutated genes in genetic disorders, inactivate the viral genome in the infected cells, generate a stop codon for inactivation and eliminate the expression of the disease-causing protein in diseases including neurodegenerative diseases, silence the oncogenic protein in cancers, mutate a splicing consensus cite to eliminate a disease causing exon, or mutate a regulatory sequence to restore a therapeutic expression/inactivation of a gene. Accordingly, the system and method disclosed in this invention can be used in correcting underlying genetic alterations in diseases including the above-mentioned genetic disorders, chronic infectious diseases, neurodegenerative diseases, and cancer. Importantly, the system and method disclosed in this invention can be used to engineer cells, for both generating research tools or for generating cell-based therapies.

### Genetic Diseases

It is estimated that over six thousand genetic diseases are caused by known genetic mutations. Correcting the underlying disease causing mutations in the pathological tissues/organs can provide alleviation or cure to the diseases. For example, cystic fibrosis affects 1 out of every 3,000 people in the US. It is caused by inheritance of a mutated CFTR gene and 70% of the patients have the same mutation, deletion of a tri-nucleotide leading to a deletion of phenylalanine at position 508 (called Δ Phe 508). Δ Phe 508 leads to the mislocation and degradation of CFTR. The system and method disclosed in this invention can be used to convert a Val 509 residue (GTT) to Phe 509 (TTT) in affected tissues (lung), thereby functionally correct the Δ Phe 508 mutation. In addition, a second suppressor mutation (such as R553Q or R553M or V510D) in the mutant Δ Phe 508 CFTR can partially restore the function of CFTR protein in somatic tissues.

### Chronic Infectious Diseases

The system and method disclosed in this invention can also be used to specifically inactivate any gene in a viral genome that is incorporated into human cells/tissues. For example, the system and method disclosed in this invention allow one to create a stop codon for early termination of translation of the essential viral genes, and thereby remediate or cure the chronic debilitating infectious diseases. For example, current AIDS therapies can reduce viral load, but cannot totally eliminate dormant HIV from positive T cells. The system and method disclosed herein can be used to permanently inactivate expression of essential HIV genes in the integrated HIV genome in human T-cells by introducing one or multiple stop codons. Another example is hepatitis B virus (HBV). The system and method disclosed here can be used to specifically inactivate essential HBV genes, which are incorporated into human genome, and silence the HBV life cycle.

### Neurodegenerative Diseases

Some neurodegenerative diseases are caused by gain-of-function mutations. For example, SOD1G93A leads to development of amyotrophic lateral sclerosis (ALS). The system and method disclosed in this invention can be used to either correct the mutation or eliminate the mutant protein expression by introducing a stop codon or by changing a splice site. For example, an alternative splicing form of Tau protein that includes exon 10 plays a causal role in Alzheimer's disease. Changing a C-G base pair at the consensus exon 10 splice site would abolish the alternative splicing version of Tau.

### Cancers

Many genes (including tumor suppressor genes, oncogenes, and DNA repair genes) contribute to the development of cancer. Mutations in these genes often lead to various cancers. Using the system and method disclosed in this invention, one can specifically target and correct these mutations. As a result, causative oncogenic proteins can be functionally repressed or their expression can be eliminated by introducing a point mutation at either the catalytic sites or splicing sites.

### Somatic Gene Knockout

In some embodiments, protein expression of a gene in somatic cells in human and non-human organisms can be eliminated by generating a pre-mature stop-codon. This approach can be used for therapeutic purpose or for generating research tools.

### Alteration of Regulatory Elements

The method could be used to change sequence of regulatory elements in DNA and RNA. Consequently, it provides an approach for altering, silencing, or activating expression of a gene through altering the various mechanisms involved in gene expression. This could be used for therapeutic purpose as well as for generating research tool.

### Stem Cell Genetic Modification

In some embodiments, cells that are reprogrammed to become different cell types can be genetically modified using the system and method disclosed in this invention. Suitable cells include, e.g., stem cells (adult stem cells, embryonic stem cells, induced Pluripotent Stem cells, mesenchymal stem cells etc. as referenced in Stem cells: past, present, and future. Zakrzewski et al. Stem Cell Res Ther. 2019 Feb. 26; 10(1):68.) and progenitor cells (e.g., cardiac progenitor cells, neural progenitor cells, etc.) or mature cells used for conversion into a different cell type (for example using the algorithm as referenced in Molecular Interaction Networks to Select Factors for Cell Conversion. Ouyang J F et al., Methods Mol Biol. 2019; 1975:333-361). Suitable cells may originate from any multicellular organism including e.g., mammals (including, e.g. rodents, humans, horses, camels, pigs), insects, avian (including, e.g. chicken, duck) etc. Suitable host cells include in vitro or ex vivo host cells, e.g., isolated host cells.

In some embodiments, the present invention can be used for targeted and precise genetic modification of cells or tissue ex vivo, correcting the underlying genetic defects. After the ex vivo correction, the tissues could be returned to the patients. Moreover, the technology can be broadly used in cell-based therapies for correcting genetic diseases.

The term “stem cell” refers herein to a cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPScs or iPS cells”.

An “embryonic stem (ES) cell” is an undifferentiated pluripotent cell which is obtained from an embryo in an early stage, such as the inner cell mass at the blastocyst stage, or produced by artificial means (e.g. nuclear transfer) and can give rise to any differentiated cell type in an embryo or an adult, including germ cells (e.g. sperm and eggs).

“Induced pluripotent stem cells (iPScs or iPS cells)” are cells generated by reprogramming a somatic cell by expressing or inducing expression of a combination of factors (herein referred to as reprogramming factors). iPS cells can be generated using fetal, postnatal, newborn, juvenile, or adult somatic cells. Factors that can be used to reprogram somatic cells to pluripotent stem cells include, for example, Oct4 (sometimes referred to as Oct3/4), Sox2, c-Myc, Klf4, Nanog, and Lin28. In some embodiments, somatic cells are reprogrammed by expressing at least two reprogramming factors, at least three reprogramming factors, at least four reprogramming factors, at least five reprogramming factors, at least six reprogramming factors, or at least seven reprogramming factors to reprogram a somatic cell to a pluripotent stem cell.

“Hematopoietic progenitor cells” or “hematopoietic precursor cells” refers to cells which are committed to a hematopoietic lineage but are capable of further hematopoietic differentiation and include hematopoietic stem cells, multipotential hematopoietic stem cells, common myeloid progenitors, megakaryocyte progenitors, erythrocyte progenitors, and lymphoid progenitors. Hematopoietic stem cells (HSCs) are multipotent stem cells that give rise to all the blood cell types including myeloid (monocytes and macrophages, granulocytes (neutrophils, basophils, eosinophils, and mast cells), erythrocytes, megakaryocytes/platelets, dendritic cells), and lymphoid lineages (T-cells, B-cells, NK-cells).

“Pluripotent stem cell” refers to a stem cell that has the potential to differentiate into all cells constituting one or more tissues or organs, or preferably, any of the three germ layers: endoderm (interior stomach lining, gastrointestinal tract, the lungs), mesoderm (muscle, bone, blood, urogenital), or ectoderm (epidermal tissues and nervous system).

As used herein, the term “somatic cell” refers to any cell other than germ cells, such as an egg, a sperm, or the like, which does not directly transfer its DNA to the next generation. Typically, somatic cells have limited or no pluripotency. Somatic cells used herein may be naturally-occurring or genetically modified.

### Cell Therapies and Ex Vivo Therapies

Various embodiments of the present invention also provide cell lines that are produced or used in accordance with any of the other embodiments of the present invention for use in therapy. In one embodiment, the present invention is directed to methods for generating therapeutic cells such as T cells engineered to express a Chimeric Antigen Receptor (CAR-T) or T Cell Receptor (TCR-T). The CAR-T/TCR-T cells may be derived from primary T cells or differentiated from stem cells. Suitable stem cells include, but are not limited to, mammalian stem cells such as human stem cells, including, but not limited to, hematopoietic, neural, embryonic, induced pluripotent stem cells (iPSC), mesenchymal, mesodermal, liver, pancreatic, muscle, and retinal stem cells. Other stems cells include, but are not limited to, mammalian stem cells such as mouse stem cells, e.g., mouse embryonic stem cells.

In various embodiments, the present invention may be used to knockdown, modify or increase the expression of a single gene or multiple genes in various types of cells or cell lines, including but not limited to cells from mammals. The technology may be used for many applications, including but not limited to knock down genes to prevent graft versus host disease by making non-host cells non-immunogenic to the host or prevent host vs graft disease by making non-host cells resistant to attack by the host. These approaches are also relevant to generating allogenic (off-the-shelf) or autologous (patient specific) cell-based therapeutics. Such genes include, but are not limited to, the T Cell Receptor (TRAC), the major histocompatibility complex (MHC class I and class II) genes, including B2M, co-receptors (HLA-F, HLA-G), genes involved in the innate immune response (MICA, MICB, HCPS), inflammation (NKBBiL, LTA, TNF, LTB, LST1, NCR3, AIF1), immune receptors (LY6), heat shock proteins (HSPA1L, HSPA1A, HSPA1B), complement cascade, regulatory receptors (NOTCH4), antigen processing (TAP, HLA-DM, HLA-DO), peptide transport (RING1), increased potency or persistence (such as PD-1, CTLA-4, FOXP3 and B7), genes involved in T cell interaction with the tumour microenvironment (including but not limited to receptors of cytokines such as TGFB, Interleukin (IL)-4, IL-7, IL-2, IL-4, as well as repressors of IL-15, IL-12, IL-18, IL-2, IFNgamma), genes involved in contributing to cytokine release syndrome (including but not limited to GMCSF), genes that code for the antigen targeted by the CAR/TCR (for example endogenous CS1 where the CAR is designed against CS1) or other genes found to be beneficial to CAR-T/TCR-T or other cell based therapeutics including but not limited to CAR-NK. CAR-B etc. See, e.g., DeRenzo et al., Genetic Modification Strategies to Enhance CAR T Cell Persistence for Patients With Solid Tumors. Front. Immunol., 15 Feb. 2019.

The technology may also be used to knock down or modify genes that are involved in fratricide of immune cells, such as T cells and NK cells, or genes that alert the immune system of a patient or animal that a foreign cell, particle or molecule has entered a patient or animal, or genes encoding proteins that are current therapeutic targets used to compromise or boost an immune response, for example, CD52 and PD1, respectively.

One application is to engineer HLA alleles of bone marrow cells to increase haplotype match. The engineered cells can be used for bone marrow transplantation for treating leukemia. Another application is to engineer the negative regulatory element of fetal hemoglobin gene in hematopoietic stem cells for treating sickle cell anemia and beta-thalassemia. The negative regulatory element will be mutated and the expression of fetal hemoglobin gene is re-activated in hematopoietic stem cells, compensating the functional loss due to mutations in adult alpha or beta hemoglobin genes. A further application is to engineer iPS cells for generating allogenic therapeutic cells for various degenerative diseases including Parkinson's disease (neuronal cell loss), Type 1 diabetes (pancreatic beta cell loss). Other exemplary applications include engineering HIV infection resistant T-Cells by inactivating CCR5 gene and other genes encoding receptors required for HIV entering cells.

The technology may also be used to generate transgenic animals that can be used as disease models or for gene function studies.

As used herein, the term “immune cells” generally includes white blood cells (leukocytes) which are derived from hematopoietic stem cells (HSC) produced in the bone marrow. Examples of immune cells include, but are not limited to, lymphocytes (T cells, B cells, and natural killer (NK) cells) and myeloid-derived cells (neutrophil, eosinophil, basophil, monocyte, macrophage, dendritic cells).

The immune cells may be isolated from subjects, particularly human subjects. The immune cells can be obtained from a subject of interest, such as a subject suspected of having a particular disease or condition, a subject suspected of having a predisposition to a particular disease or condition, or a subject who is undergoing therapy for a particular disease or condition. Immune cells can be collected from any location in which they reside in the subject including, but not limited to, blood, cord blood, spleen, thymus, lymph nodes, and bone marrow. The isolated immune cells may be used directly, or they can be stored for a period of time, such as by freezing.

The immune cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the immune cells are enriched, isolated, and/or purified may be isolated from both living and non-living subjects, wherein the non-living subjects are organ donors. In particular embodiments, the immune cells are isolated from blood, such as peripheral blood or cord blood. In some aspects, immune cells isolated from cord blood have enhanced immunomodulation capacity, such as measured by CD4- or CD8-positive T cell suppression. In specific aspects, the immune cells are isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune cell activity. Thus, the cells can be autologous to the subject in need of therapy. Alternatively, the population of immune cells can be obtained from a donor, preferably a histocompatibility matched donor. The immune cell population can be harvested from the peripheral blood, cord blood, bone marrow, spleen, or any other organ/tissue in which immune cells reside in said subject or donor. The immune cells can be isolated from a pool of subjects and/or donors, such as from pooled cord blood.

When the population of immune cells is obtained from a donor distinct from the subject, the donor is preferably allogeneic, provided the cells obtained are subject-compatible in that they can be introduced into the subject. Allogeneic donor cells are may or may not be human-leukocyte-antigen (HLA)-compatible. To be rendered subject-compatible, allogeneic cells can be treated to reduce immunogenicity.

In some embodiment, the immune cells may be T cells (e.g., regulatory T cells, CD4⁺ T cells, CD⁸ T cells, or gamma-delta T cells), NK cells, invariant NK cells, NKT cells, stem cells (e.g., mesenchymal stem cells (MSCs) or induced pluripotent stem (iPSC) cells). In some embodiments, the cells are monocytes or granulocytes, e.g., myeloid cells, macrophages, neutrophils, dendritic cells, mast cells, eosinophils, and/or basophils. Also provided herein are methods of producing and engineering the immune cells as well as methods of using and administering the cells for adoptive cell therapy, in which case the cells may be autologous or allogeneic. Thus, the immune cells may be used as immunotherapy, such as to target cancer cells.

### Genetic Editing in Animals and Plants

The system and method described above can be used to generate a transgenic non-human animal or plant having one or more genetic modification of interest. In some embodiments, the transgenic non-human animal is homozygous for the genetic modification. In some embodiments, the transgenic non-human animal is heterozygous for the genetic modification. In some embodiments, the transgenic non-human animal is a vertebrate, for example, a fish (e.g., zebra fish, gold fish, puffer fish, cave fish, etc.), an amphibian (frog, salamander, etc.), a bird (e.g., chicken, turkey, etc.), a reptile (e.g., snake, lizard, etc.), a mammal (e.g., an ungulate, e.g., a pig, a cow, a goat, a sheep, etc.; a lagomorph (e.g., a rabbit); a rodent (e.g., a rat, a mouse); a non-human primate.

The invention can be used for treating diseases in animals in a way similar to those for treating diseases in humans as described above. Alternatively, it can be used to generate knock-in animal disease models bearing specific genetic mutation for purposes of research, drug discovery, and target validation. The system and method described above can also be used for introduction of point mutations to ES cells or embryos of various organisms, for purpose of breeding and improving animal stocks and crop quality.

Methods of introducing exogenous nucleic acids into plant cells are well known in the art. Suitable methods include viral infection (such as double stranded DNA viruses), transfection, conjugation, protoplast fusion, electroporation, particle gun technology, calcium phosphate precipitation, direct microinjection, silicon carbide whiskers technology, Agrobacterium-mediated transformation and the like. The choice of method is generally dependent on the type of cell being transformed and the circumstances under which the transformation is taking place (i.e., in vitro, ex vivo, or in vivo).

### Kit

This invention further provides kits containing reagents for performing the above-described methods, including CRISPR/Cas guided target binding or correction reaction. To that end, one or more of the reaction components, e.g., RNAs, Cas proteins, fusion effector proteins and related nucleic acids, for the methods disclosed herein can be supplied in the form of a kit for use. In one embodiment, the kit comprises a CRISPR protein or a nucleic acid encoding the Cas protein, effector protein, one or more of an RNA scaffold described above, a set of RNA molecules described above. In other embodiments, the kit can include one or more other reaction components. In such a kit, an appropriate amount of one or more reaction components is provided in one or more containers or held on a substrate.

Examples of additional components of the kits include, but are not limited to, one or more host cells, one or more reagents for introducing foreign nucleotide sequences into host cells, one or more reagents (e.g., probes or PCR primers) for detecting expression of the RNA or protein or verifying the target nucleic acid's status, and buffers or culture media for the reactions (in 1× or concentrated forms). The kit may also include one or more of the following components: supports, terminating, modifying or digestion reagents, osmolytes, and an apparatus for detection.

The reaction components used can be provided in a variety of forms. For example, the components (e.g., enzymes, RNAs, probes and/or primers) can be suspended in an aqueous solution or as a freeze-dried or lyophilized powder, pellet, or bead. In the latter case, the components, when reconstituted, form a complete mixture of components for use in an assay. The kits of the invention can be provided at any suitable temperature. For example, for storage of kits containing protein components or complexes thereof in a liquid, it is preferred that they are provided and maintained below 0° C., preferably at or below −20° C., or otherwise in a frozen state.

A kit or system may contain, in an amount sufficient for at least one assay, any combination of the components described herein. In some applications, one or more reaction components may be provided in pre-measured single use amounts in individual, typically disposable, tubes or equivalent containers. With such an arrangement, an RNA-guided reaction can be performed by adding a target nucleic acid, or a sample or cell containing the target nucleic acid, to the individual tubes directly. The amount of a component supplied in the kit can be any appropriate amount and may depend on the target market to which the product is directed. The container(s) in which the components are supplied can be any conventional container that is capable of holding the supplied form, for instance, microfuge tubes, microtiter plates, ampoules, bottles, or integral testing devices, such as fluidic devices, cartridges, lateral flow, or other similar devices.

The kits can also include packaging materials for holding the container or combination of containers. Typical packaging materials for such kits and systems include solid matrices (e.g., glass, plastic, paper, foil, micro-particles and the like) that hold the reaction components or detection probes in any of a variety of configurations (e.g., in a vial, microtiter plate well, microarray, and the like). The kits may further include instructions recorded in a tangible form for use of the components.

### Definition

A nucleic acid or polynucleotide refers to a DNA molecule (for example, but not limited to, a cDNA or genomic DNA) or an RNA molecule (for example, but not limited to, an mRNA), and includes DNA or RNA analogs. A DNA or RNA analog can be synthesized from nucleotide analogs. The DNA or RNA molecules may include portions that are not naturally occurring, such as modified bases, modified backbone, deoxyribonucleotides in an RNA, etc. The nucleic acid molecule can be single-stranded or double-stranded.

The term “isolated” when referring to nucleic acid molecules or polypeptides means that the nucleic acid molecule or the polypeptide is substantially free from at least one other component with which it is associated or found together in nature.

As used herein, the term “guide RNA” generally refers to an RNA molecule (or a group of RNA molecules collectively) that can bind to a CRISPR protein and target the CRISPR protein to a specific location within a target DNA. A guide RNA can comprise two segments: a DNA-targeting guide segment and a protein-binding segment. The DNA-targeting segment comprises a nucleotide sequence that is complementary to (or at least can hybridize to under stringent conditions) a target sequence. The protein-binding segment interacts with a CRISPR protein, such as a Cas9 or Cas9 related polypeptide. These two segments can be located in the same RNA molecule or in two or more separate RNA molecules. When the two segments are in separate RNA molecules, the molecule comprising the DNA-targeting guide segment is sometimes referred to as the CRISPR RNA (crRNA), while the molecule comprising the protein-binding segment is referred to as the trans-activating RNA (tracrRNA).

As used herein, the term “target nucleic acid” or “target” refers to a nucleic acid containing a target nucleic acid sequence. A target nucleic acid may be single-stranded or double-stranded, and often is double-stranded DNA. A “target nucleic acid sequence,” “target sequence” or “target region,” as used herein, means a specific sequence or the complement thereof that one wishes to bind to or modify using a CRISPR system. A target sequence may be within a nucleic acid in vitro or in vivo within the genome of a cell, which may be any form of single-stranded or double-stranded nucleic acid.

A “target nucleic acid strand” refers to a strand of a target nucleic acid that is subject to base-pairing with a guide RNA as disclosed herein. That is, the strand of a target nucleic acid that hybridizes with the crRNA and guide sequence is referred to as the “target nucleic acid strand.” The other strand of the target nucleic acid, which is not complementary to the guide sequence, is referred to as the “non-complementary strand.” In the case of double-stranded target nucleic acid (e.g., DNA), each strand can be a “target nucleic acid strand” to design crRNA and guide RNAs and used to practice the method of this invention as long as there is a suitable PAM site.

As used herein, the term “derived from” refers to a process whereby a first component (e.g., a first molecule), or information from that first component, is used to isolate, derive or make a different second component (e.g., a second molecule that is different from the first). For example, the mammalian codon-optimized Cas9 polynucleotides are derived from the wild type Cas9 protein amino acid sequence. Also, the variant mammalian codon-optimized Cas9 polynucleotides, including the Cas9 single mutant nickase (nCas9, such as nCas9D10A) and Cas9 double mutant null-nuclease (dCas9, such as dCas9 D10A H840A), are derived from the polynucleotide encoding the wild type mammalian codon-optimized Cas9 protein.

As used herein the term “wild type” is a term of the art understood by skilled persons and means the typical form of an organism, strain, gene or characteristic as it occurs in nature as distinguished from mutant or variant forms.

As used herein, the term “variant” refers to a first composition (e.g., a first molecule), that is related to a second composition (e.g., a second molecule, also termed a “parent” molecule). The variant molecule can be derived from, isolated from, based on or homologous to the parent molecule. For example, the mutant forms of mammalian codon-optimized Cas9 (hspCas9), including the Cas9 single mutant nickase and the Cas9 double mutant null-nuclease, are variants of the mammalian codon-optimized wild type Cas9 (hspCas9). The term variant can be used to describe either polynucleotides or polypeptides.

As applied to polynucleotides, a variant molecule can have entire nucleotide sequence identity with the original parent molecule, or alternatively, can have less than 100% nucleotide sequence identity with the parent molecule. For example, a variant of a gene nucleotide sequence can be a second nucleotide sequence that is at least 50%, 60%, 70%, 80%, 90%, 95%, 98%, 99% or more identical in nucleotide sequence compare to the original nucleotide sequence. Polynucleotide variants also include polynucleotides comprising the entire parent polynucleotide, and further comprising additional fused nucleotide sequences. Polynucleotide variants also includes polynucleotides that are portions or subsequences of the parent polynucleotide, for example, unique subsequences (e.g., as determined by standard sequence comparison and alignment techniques) of the polynucleotides disclosed herein are also encompassed by the invention.

In another aspect, polynucleotide variants include nucleotide sequences that contain minor, trivial or inconsequential changes to the parent nucleotide sequence. For example, minor, trivial or inconsequential changes include changes to nucleotide sequence that (i) do not change the amino acid sequence of the corresponding polypeptide, (ii) occur outside the protein-coding open reading frame of a polynucleotide, (iii) result in deletions or insertions that may impact the corresponding amino acid sequence, but have little or no impact on the biological activity of the polypeptide, (iv) the nucleotide changes result in the substitution of an amino acid with a chemically similar amino acid. In the case where a polynucleotide does not encode for a protein (for example, a tRNA or a crRNA or a tracrRNA), variants of that polynucleotide can include nucleotide changes that do not result in loss of function of the polynucleotide. In another aspect, conservative variants of the disclosed nucleotide sequences that yield functionally identical nucleotide sequences are encompassed by the invention. One of skill will appreciate that many variants of the disclosed nucleotide sequences are encompassed by the invention.

As applied to proteins, a variant polypeptide can have entire amino acid sequence identity with the original parent polypeptide, or alternatively, can have less than 100% amino acid identity with the parent protein. For example, a variant of an amino acid sequence can be a second amino acid sequence that is at least 50%, 60%, 70%, 80%, 90%, 95%, 96%, 97%, 98%, 99% or more identical in amino acid sequence compared to the original amino acid sequence.

Polypeptide variants include polypeptides comprising the entire parent polypeptide, and further comprising additional fused amino acid sequences. Polypeptide variants also includes polypeptides that are portions or subsequences of the parent polypeptide, for example, unique subsequences (e.g., as determined by standard sequence comparison and alignment techniques) of the polypeptides disclosed herein are also encompassed by the invention.

In another aspect, polypeptide variants include polypeptides that contain minor, trivial or inconsequential changes to the parent amino acid sequence. For example, minor, trivial or inconsequential changes include amino acid changes (including substitutions, deletions and insertions) that have little or no impact on the biological activity of the polypeptide, and yield functionally identical polypeptides, including additions of non-functional peptide sequence. In other aspects, the variant polypeptides of the invention change the biological activity of the parent molecule, for example, mutant variants of the Cas9 polypeptide that have modified or lost nuclease activity. One of skill will appreciate that many variants of the disclosed polypeptides are encompassed by the invention.

In some aspects, polynucleotide or polypeptide variants of the invention can include variant molecules that alter, add or delete a small percentage of the nucleotide or amino acid positions, for example, typically less than about 10%, less than about 5%, less than 4%, less than 2% or less than 1%.

As used herein, the term “conservative substitutions” in a nucleotide or amino acid sequence refers to changes in the nucleotide sequence that either (i) do not result in any corresponding change in the amino acid sequence due to the redundancy of the triplet codon code, or (ii) result in a substitution of the original parent amino acid with an amino acid having a chemically similar structure. Conservative substitution tables providing functionally similar amino acids are well known in the art, where one amino acid residue is substituted for another amino acid residue having similar chemical properties (e.g., aromatic side chains or positively charged side chains), and therefore does not substantially change the functional properties of the resulting polypeptide molecule.

The following are groupings of natural amino acids that contain similar chemical properties, where a substitution within a group is a “conservative” amino acid substitution. This grouping indicated below is not rigid, as these natural amino acids can be placed in different grouping when different functional properties are considered. Amino acids having nonpolar and/or aliphatic side chains include: glycine, alanine, valine, leucine, isoleucine and proline. Amino acids having polar, uncharged side chains include: serine, threonine, cysteine, methionine, asparagine and glutamine Amino acids having aromatic side chains include: phenylalanine, tyrosine and tryptophan. Amino acids having positively charged side chains include: lysine, arginine and histidine. Amino acids having negatively charged side chains include: aspartate and glutamate.

A “Cas9 mutant” or “Cas9 variant” refers to a protein or polypeptide derivative of the wild type Cas9 protein such as S. pyogenes Cas9 protein (i.e., SEQ ID NO: 1), e.g., a protein having one or more point mutations, insertions, deletions, truncations, a fusion protein, or a combination thereof. It retains substantially the RNA targeting activity of the Cas9 protein. The protein or polypeptide can comprise, consist of, or consist essentially of a fragment of SEQ ID NO: 1. In general, the mutant/variant is at least 50% (e.g., any number between 50% and 100%, inclusive) identical to SEQ ID NO: 1. The mutant/variant can bind to an RNA molecule and be targeted to a specific DNA sequence via the RNA molecule, and may additional have a nuclease activity. Examples of these domains include RuvC like motifs (aa. 7-22, 759-766 and 982-989 in SEQ ID NO: 1) and HNH motif (aa 837-863). See Gasiunas et al., Proc Natl Acad Sci USA. 2012 Sep. 25; 109(39): E2579-E2586 and WO2013176772.

“Complementarity” refers to the ability of a nucleic acid to form hydrogen bond(s) with another nucleic acid sequence by either traditional Watson-Crick base-pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques in Biochemistry and Molecular Biology-Hybridization with Nucleic Acid Probes Part I, Second Chapter “Overview of principles of hybridization and the strategy of nucleic acid probe assay”, Elsevier, N.Y.

“Hybridization” or “hybridizing” refers to a process where completely or partially complementary nucleic acid strands come together under specified hybridization conditions to form a double-stranded structure or region in which the two constituent strands are joined by hydrogen bonds. Although hydrogen bonds typically form between adenine and thymine or uracil (A and T or U) or cytidine and guanine (C and G), other base pairs may form (e.g., Adams et al., The Biochemistry of the Nucleic Acids, 11th ed., 1992).

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, pegylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The term “fusion polypeptide” or “fusion protein” means a protein created by joining two or more polypeptide sequences together. The fusion polypeptides encompassed in this invention include translation products of a chimeric gene construct that joins the nucleic acid sequences encoding a first polypeptide, e.g., an RNA-binding domain, with the nucleic acid sequence encoding a second polypeptide, e.g., an effector domain, to form a single open-reading frame. In other words, a “fusion polypeptide” or “fusion protein” is a recombinant protein of two or more proteins which are joined by a peptide bond or via several peptides. The fusion protein may also comprise a peptide linker between the two domains.

The term “linker” refers to any means, entity or moiety used to join two or more entities. A linker can be a covalent linker or a non-covalent linker. Examples of covalent linkers include covalent bonds or a linker moiety covalently attached to one or more of the proteins or domains to be linked. The linker can also be a non-covalent bond, e.g., an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, the domains can be modified by oxidation, hydroxylation, substitution, reduction etc. to provide a site for coupling. Methods for conjugation are well known by persons skilled in the art and are encompassed for use in the present invention. Linker moieties include, but are not limited to, chemical linker moieties, or for example a peptide linker moiety (a linker sequence). It will be appreciated that modification which do not significantly decrease the function of the RNA-binding domain and effector domain are preferred.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

The terms “subject” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed. In some embodiments, a subject may be an invertebrate animal, for example, an insect or a nematode; while in others, a subject may be a plant or a fungus.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising a therapeutic agent, such as a cell, or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards. As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

As used herein, the term “contacting,” when used in reference to any set of components, includes any process whereby the components to be contacted are mixed into same mixture (for example, are added into the same compartment or solution), and does not necessarily require actual physical contact between the recited components. The recited components can be contacted in any order or any combination (or sub-combination) and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C. “Contacting” a target nucleic acid or a cell with one or more reaction components, such as an Cas protein or guide RNA, includes any or all of the following situations: (i) the target or cell is contacted with a first component of a reaction mixture to create a mixture; then other components of the reaction mixture are added in any order or combination to the mixture; and (ii) the reaction mixture is fully formed prior to mixture with the target or cell.

The term “mixture” as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not spatially distinct. In other words, a mixture is not addressable.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention. The term “about” generally refers to plus or minus 10% of the indicated number. For example, “about 10%” may indicate a range of 9% to 11%, and “about 20” may mean from 18-22. Other meanings of “about” may be apparent from the context, such as rounding off, so, for example “about 1” may also mean from 0.5 to 1.4.

### EXAMPLES

### Example 1 Material and Methods

This example describes material and methods used in Examples 2-12 bellow

### Bacterial Strains

E. coli DH5a competent cells were purchased from THERMO FISHER (Cat. No. 18265017) and were used for general cloning purposes. E. coli MG1655 strain, used for rpoB gene targeting, was a kind gift from Dr. Stanley Qi (Stanford University). MG1655 cells were made competent using a standard CaCl₂) protocol.

### Bacterial Expression Plasmids

PgRNA-bacteria (pUC19, ampicillin resistant; ADDGENE plasmid #44251) was engineered to include two offset BbsI restriction sites for guiding sequence cloning, as well as 1 or 2 MS2 stem loop sequences at the 3′ end. These modifications were introduced using standard gene synthesis services (GENEWIZ; South Plainfield, N.J., USA). The synthesized cassettes were cloned into pUC19 backbone using SpeI and HindIII restriction sites. The effector modules (AID-linker-MCP) were cloned into a pCDFDuet empty vector (DF13, streptomycin resistant; ADDGENE plasmid #49796) using BglII and BamHI restriction sites. dCas9-bacteria plasmid (p15A, chloramphenicol resistant; ADDGENE #44249), and pwtCas9-bacteria (p15A; ADDGENE #44250) were used to generate nCas9_{D10A} and nCas9_{H840A} nickases by swapping portions of the wild type HNH and RuvC active sites, respectively, from pwtCas9 to dCas9. HNH domain was cloned using Acc65I and BamHI restriction sites. RuvC domain was cloned using XbaI and NheI restriction sites. Cas9 and effector constructs are under the control of a tetracycline inducible promoter.

Bacterial gRNA Design

RpoB targeting gRNAs were designed manually on SNAPGENE VIEWER (GSL BIOTECH), on or near the rifampicin resistance determining region (RRDR) of E. coli's rpoB gene. (23) gRNA sequences and PAMs are summarized in Table S1. Guiding sequences were designed to have 5′ overhangs compatible to the overhangs left by BbsI digestion (i.e., Fwd 5′-CTAGN₂₀-3′ (SEQ ID NO: 84), Rev 5′-AAACN₂₀-3′(SEQ ID NO: 85), where N₂₀ is the programmable guiding sequence and must be complementary between Fwd and Rev oligos).

TABLE S1
rpoB targeting gRNA sequences

| | Target | Target | | |
|---|---|---|---|---|
| Name | strand | gene | Sequence (SEQ ID NO) | PAM |
| TS1 | Template | rpoB | GCAGCAGTGAAAGAGTTCTT | CGG |
| | | | (86) | |
| TS2 | Template | rpoB | CAGCCAGCTGTCTCAGTTTA | TGG |
| | | | (87) | |
| TS3 | Template | rpoB | AAACGTCGTATCTCCGCACT | CGG |
| | | | (88) | |
| TS4 | Template | rpoB | CGTATCTCCGCACTCGGCCC | AGG |
| | | | (89) | |

### Bacterial Treatments:

Chemically competent E. coli MG1655 cells were transformed with 9 ng of a 1:1:1 combination of the appropriate plasmids encoding for specific gRNA (ampicillin), AID_MCP (streptomycin) and Cas9 (chloramphenicol) constructs. After transformation, cells were selected overnight in liquid LB media containing working concentrations of ampicillin, streptomycin and chloramphenicol. The day after, cells are diluted in selective media supplemented with 3 μM tetracycline to induce expression of the protein coding modules. After overnight growth, OD is measured, and serial dilutions are performed to plate 10⁸-10³ cells in rifampicin containing LB agar. Plates are incubated at 37° C. and monitored for 48 h. Surviving fraction is calculated by counting the surviving colonies divided by the number of cells plated.

### Mutational Analysis in Bacterial Experiments

Genomic DNA from 8 to 12 colonies from appropriate experiments was extracted. The target region of the rpoB gene (i.e., RRDR region) was PCR amplified, and the purified PCR products were sequenced using Sanger chemistry at GENEWIZ (South Plainfield, N.J., USA). Primer sequences are summarized in the table below.

TABLE S6
Primers used in this study:

| | | SEQ ID | | SEQ |
|---|---|---|---|---|
| Gene | Fwd primer | NO | Rev Primer | ID NO |
| rpoB | TTGGCGAAATGGCGGAAA | 90 | CACCGACGGATACCACCTGCTG | 91 |
| | ACC | | | |
| site2 | CCTGGCTGAGCTAACTGT | 92 | GTCAAACTGTGCGTATGACATCATC | 93 |
| | GACAG | | AG | |
| site3 | GCATGCATTTGTAGGCTT | 94 | GCCCCTGTCTAGGAAAAGCTGTC | 95 |
| | GATGC | | | |
| site4 | CTGGGTGGAAGGAAGGGA | 96 | TCAACCCGAACGGAGACACACAC | 97 |
| | GGAAG | | | |
| S2O2 | CTGTTTGCCTTAGGAGAG | 98 | CTCTGAACACAAGCCTTTCTCCAGG | 99 |
| | GCCAGAG | | G | |
| S3O1 | GACCTGGAGAAGCATGAA | 100 | CATGGTGTGCCTGTCACTGTACTTG | 101 |
| | CCAGTC | | | |
| S3O02 | GAGGTCCAAGGAGGCCTA | 102 | GGGAAGGAGACTTAGTGAGACTTGA | 103 |
| | TGCAG | | AACC | |
| S3O3 | CTGAGCGCACATCCC | 104 | CTGCTACTGGAGCACACCCCAAG | 105 |
| | TTGTCTCTC | | | |
| S4O1 | CATAGCTGGGGCTGAAGA | 106 | CTCCTCGGAGTCCTCAAGTATCACT | 107 |
| | TCCCTAG | | G | |
| S4O2 | GTGCTTGGGTTGCTTTGG | 108 | GTTGCTTTGGCAATGGAGGCATTG | 109 |
| | CAATG | | | |
| S4O4 | GTGAAGAACTCCAGGG | 110 | CACCACCTCTTCCATCTGCCTTGTC | 111 |
| | GCAATCTGAAG | | | |
| EGFP_ | CTTCAAGGAGGACGGCAA | 112 | TGTTCTGCTGGTAGTGGTCGGC | 113 |
| TS1 | CATCC | | | |
| EGFP_ | CGGCATCAAGGTGAACTT | 114 | CTCGTTGGGGTCTTTGCTCAGG | 115 |
| TS2 | CAAGATCC | | | |

### Mammalian Expression Plasmids.

To generate ^{A}CRCn, ^{A}CRCnu and ^{A1}CRCnu multicistronic constructs, AID_MCP or APOBEC 1_MCP fusions were synthesized at GENWIZ (South Plainfield, N.J., USA) and cloned upstream of nCas9_UGI (13). The two modules are separated by a self-cleavable T2A peptide. To generate second-generation ^{A}CRCnu.2, the constructs were codon optimized and an additional UGI copy was included downstream con Cas9 (29). To generate gRNA_2×MS2 vector, the gRNA scaffold fused to 2 MS2 loops (15) was synthesized at GENEWIZ (South Plainfield, N.J., USA) and cloned into phU6_gRNA (ADDGENE plasmid #53188) (49). nfEGFP gene harbors an A→G mutation at nucleotide 200 of the GFP gene, and was synthesized at GENEWIZ (South Plainfield, N.J., USA) and cloned into pCMV_Sports6 vector using SalI and NotI restriction sites.

gRNA Design

Targeting gRNAs were designed manually on SNAPGENE VIEWER (GSL BIOTECH).

All gRNAs used in this study are described in Tables S3 and S4.

TABLE S3
EGFP targeting gRNA sequences

| Name | Target strand | Sequence (SEQ ID NO) | PAM |
|---|---|---|---|
| NT1 | Non-template | CGCAGGTCAGGGTGGTCACG (116) | AGG |
| TS1 | Template strand | CAAGCAGAAGAACGGCATCA (117) | AGG |

Note:
Target C is underlined

TABLE S4
Target sequence and genomic locations of endogenous human loci

| | | | Genomic coordinates |
|---|---|---|---|
| Name | Sequence (SEQ ID NO) | PAM | (GRCh38/hg38) |
| Site2 | GAACACAAAGCATAGACTGC (118) | GGG | chr5:87,944,780-87,944,799 |
| Site3 | GGCCCAGACTGAGCACGTGA (119) | TGG | chr9:107,422,339-107,422,358 |
| Site4 | GGCACTGCGGCTGGAGGTGG (120) | GGG | chr20:32,761,950-32,761,969 |
| PDCD1_TS1 | CGCAGATCAAAGAGAGCCTG (121) | CGG | chr2:241,852,643-241,852,662 |

### Cell Culture

HEK 293T cells were purchased from ATCC (CRL-3216). Transgenic EGFP reporters were generated by standard lentiviral transduction on HEK 293T and selected with puromycin. Cells expressing GFP variants were obtained by limiting dilution. Cells were grown and maintained at 37° C. and 5% CO2 in Dulbecco's Modified Eagle Medium (DMEM, THERMOFISHER), supplemented with 10% fetal bovine serum, 1× Glutamine (THERMOFISHER) and 1× Antibiotic-Antimycotic (THERMOFISHER).

### Treatments

HEK 293T and its derivatives nf2.16 or 293_GFP cells were plated in 6-well plates the day before experiments (3.5×10⁵ cells per well). Transfections were performed on cells 75-85% confluent, with a total of 2 μg of a combination of DNA from CRC and gRNA constructs in a 3:1 ratio, respectively. LIPOFECTAMINE 2000 (THERMOFISHER) or LIPOFECTAMINE 3000 was used as transfection reagent, following manufacturer's procedure. When appropriate, 72 hours after transfection, fluorescent pictures were taken, GFP signal was quantified by flow cytometry in a Gallios Flow Cytometer instrument (BECKMAN COULTER) at the Rutgers University's Flow Cytometry core facility. To observe GFP loss by fluorescence microcopy and flow cytometry, in the knockout experiments, cells were passaged and cultured for additional 96 hours to allow GFP turnover in treated cells. After treatments, DNA was purified for downstream analysis using DNEASY BLOOD AND TISSUE KIT (QIAGEN).

### FACS Analysis

nf2.16 cells were treated with ^{A}CRCnu/nfEGFP_NTE 72 hours after transfection, GFP positive cells were sorted at the Rutgers University's Flow Cytometry core facility on a BECKMAN COULTER MOFLO XDP Cell Sorter instrument following manufacturer instructions. Sorted cells expressing wild type GFP were cultured, DNA was harvested using DNEASY BLOOD AND TISSUE KIT (QIAGEN), and the target region was amplified by PCR followed by Sanger sequencing at GENEWIZ (New Jersey, USA). Primers used for PCR were the same as the ones used for high throughput sequencing analysis (see below and Table S6).

### Whole-Exome Sequencing Analysis (WES)

WES was carried out by GENEWIZ (South Plainfield, N.J., USA). The WES libraries were constructed using AGILENT SURESELECT HUMAN ALL EXON (V6 r2) library prep kit and sequenced using ILLUMINA HISEQ with the pair-end 2×150 bp format. To estimate potential CRC off-target activity, raw data was analyzed as follows:

Variant Calling and Alternative Reference Construction

WES raw reads were aligned to the human reference genome (hg38) with BWA (version 0.7.15). Variants were identified using GENOME ANALYSIS TOOL KIT (GATK) version 3.8 roughly following the GATK best practices. Briefly, duplicate reads were first marked with Picard MARKDUPLICATES. BASERECALIBRATOR was used to recalibrate base quality, and HAPLOTYPECALLER was then used to call variants on each sample followed by joint genotyping with GENOTYPEGVCFS. The detected variants in the resulting VCF file were further recalibrated with VARIANTRECALIBRATOR.

In the downstream analysis, inventors only focused on the exonic regions as defined in “SURESELECT HUMAN ALL EXON V6 r2”. In the analysis, the overlapping regions were merged using function bedtools merge.

To construct alternate reference based on the parental cell line T6, inventors extracted all variants that are genotyped in T6. GATK3.8 FASTAALTERNATEREFERENCEMAKER was used with default options to construct alternate reference sequence in exonic regions specified in the merged exon-target file.

Motif Definition and Mutation Analysis

AID “WRCH” binding motifs represent a product of [‘AT’,‘AG’,‘C’,‘ACT’], and coordinates for any such four consecutive nucleotides were stored. Inventors used python to identify and extract genomic locations of WRCH motifs within reference FASTA sequence (either hg38 or alternate reference). Reference FASTA sequence was also scanned for sequences complementary to “WRCH”, i.e. “DGYW”, and given by the product of [‘AGT’,‘G’,‘CT’,‘AT’]. A non-WRCH-motif was defined as a four-nucleotide sequence with a C on the third position, which is not WRCH. Similarly, non-DGYW-motif is any four-nucleotide sequence with a G on the second position and not DGYW. In total, there are 12 possible WRCH motifs, 12 DGYW motifs, 52 non-WRCH-motifs, and 52 non-DGYW-motifs. In the mutation analysis, WRCH and DGYW categories were examined separately. When looking for potential AID-derived mutated sites a C>T change is categorized as a WRCH motif mutation or a non-WRCH-motif mutation based on its surrounding bases. Similarly, G>A changes are categorized as a DGYW motif mutation or a non-DGYW-motif mutation based on their surrounding bases.

Putative CRISPR Off-Target Regions

Reference genome hg38 was scanned for the putative loci of CRISPR gRNA targeting using CCTop (https://crispr.cos.uni-heidelberg.de/) and CRISPRDesign (http://crispr.mit.edu/). Together 54 putative off target regions were obtained and variants within these regions extracted.

### High-Throughput Sequencing Analysis

Sequences of primers used in this study are summarized in Table S6. All PCR amplifications were performed with high fidelity PHUSION HOT START DNA Polymerase (NEW ENGLAND BIOLABS), as per manufacturer's instructions. PCR products were purified with QIAQUICK PCR PURIFICATION KIT (QIAGEN) and submitted to GENEWIZ (South Plainfield, N.J., USA) for high-throughput sequencing analysis. Data analysis, specifically frequency of single nucleotide polymorphisms (SNPs) and insertions/deletions (indels), was performed by GENEWIZ personnel using a proprietary pipeline. Sequencing output was used to generate SNP and INDEL frequency figures.

### Exome-Wide Sequencing Analysis

DNA Library Preparation and HiSeq Sequencing Initial DNA sample quality assessment, DNA library preparation, sequencing and bioinformatics analysis were conducted at GENEWIZ, Inc. (South Plainfield, N.J., USA). Genomic DNA samples were quantified using QUBIT 2.0 Fluorometer (LIFE TECHNOLOGIES, Carlsbad, Calif., USA) and DNA integrity was checked with 0.6% agarose gel with 50 ng sample loaded in each lane. SURESELECTXT EXOME ENRICHMENT SYSTEM for ILLUMINA Paired-End Multiplexed Sequencing Library and SURESELECT HUMAN ALL EXON V5 bait library were used for target enrichment DNA library preparation following the manufacturer's recommendations (AGILENT, Santa Clara, Calif., USA) and the standard low-input protocol (for 200 ng starting material). Briefly, the genomic DNA was fragmented by acoustic shearing with a COVARIS LE200 Focused Ultra-sonicator instrument. Fragmented DNAs were cleaned up and end repaired, as well as adenylated at the 3′ ends. Adapters were ligated to the DNA fragments, and adapter ligated DNA fragments were enriched with limited cycle PCR. Adapter-ligated DNA fragments were validated using AGILENT TAPESTATION (AGILENT TECHNOLOGIES, Palo Alto, Calif., USA), and quantified using QUBIT 2.0 Fluorometer. 750 ng adapter-ligated DNA fragments were hybridized with biotinylated RNA baits at 65 C for 24 hours. The hybrid DNAs were captured by streptavidin-coated magnetic beads. After extensive wash, the captured DNAs were amplified and indexed with ILLUMINA indexing primers. Post-captured DNA libraries were validated using AGILENT TAPESTATION and quantified using QUBIT 2.0 Fluorometer and Real-Time PCR (APPLIED BIOSYSTEMS, Carlsbad, Calif., USA).

ILLUMINA reagents and kits for DNA library sequencing cluster generation and sequencing were used for enrichment DNA sequencing. Post-captured DNA libraries were multiplexed in equal molar mass, and pooled DNA libraries were clustered on two lanes of a flow cell, using the cBOT from ILLUMINA. After clustering, the flow cell was loaded on the ILLUMINA HISEQ instrument according to manufacturer's instructions. The samples were sequenced using a 2×150 pairedend (PE) configuration. Image analysis and base calling was conducted by the HiSeq Control Software (HCS 2.0) on the HISEQ instrument.

### High Throughput Sequencing Analysis

Library preparation. DNA Library Preparation and ILLUMINA Sequencing DNA library preparations, sequencing reactions, and initial bioinformatics analysis were conducted at GENEWIZ, Inc. (South Plainfield, N.J., USA). DNA amplicon was indexed and enriched by limited cycle PCR. The DNA library was validated using TapeStation (Agilent Technologies, Palo Alto, Calif., USA), and was quantified using QUBIT 2.0 Fluorometer and real time PCR (APPLIED BIOSYSTEMS, Carlsbad, Calif., USA). The pooled DNA libraries were loaded on the ILLUMINA instrument according to manufacturer's instructions. The samples were sequenced using a 2×250 paired-end (PE) configuration. Image analysis and base calling were conducted by the ILLUMINA CONTROL SOFTWARE (HCS) on the ILLUMINA instrument.

Data analysis. The raw ILLUMINA reads were checked for adapters and quality via FASTQC. The raw ILLUMINA sequence reads were trimmed of their adapters and nucleotides with poor quality using TRIMMOMATIC v. 0.36. Paired sequence reads were then merged to form a single sequence if the forward and reverse reads were able to overlap and the overlapped region was identical using the reformat function within bbmap. The merged reads were aligned to the reference sequence and variant detection was performed using GENEWIZ proprietary AMPLICON-EZ program

### Example 2 CRC System: A Modular Base Editing Platform

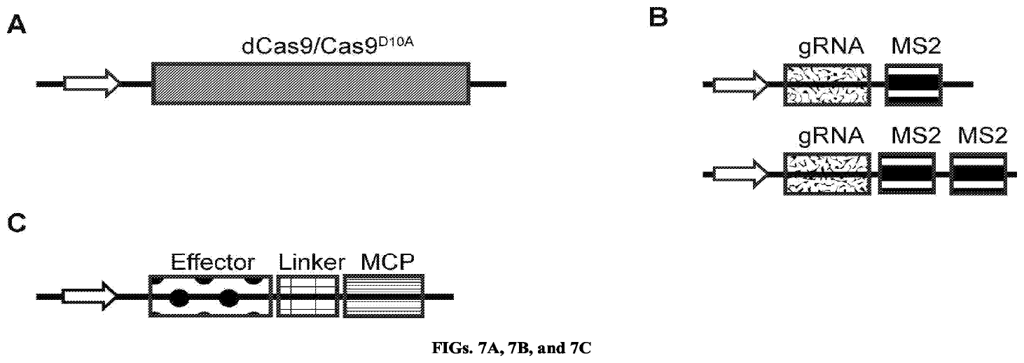

The CRC base editing system consists three functional modules illustrated in FIGS. 1A and 1 B: (1) a nuclease deficient Cas9 protein; (2) a programmable chimeric RNA scaffold containing gRNA (for sequence recognition [2.1] and Cas9 binding [2.2]) and a recruiting RNA aptamer (for effector module recruitment [2.3]); and (3) the effector module consisting a cytidine deaminase (effector [3.1]) fused to the RNA aptamer ligand, a small RNA binding protein [3.2] that specifically interacts with the recruiting RNA-aptamer. An initial prototype system consisted of bacterial vectors expressing catalytically dead Cas9 protein (dCas9, containing mutations D10A and H840A abrogating its nuclease activity), an RNA aptamer derived from the operator stemloop of bacteriophage MS2 (MS2) synthetically fused to the 3′ end of gRNA scaffold, and human activation induced cytidine deaminase (AID) fused to MS2 coat protein (MCP) which interacts with MS2 (FIG. 7). In FIG. 1, the effector is shown as a monomer, however in cells AID or other effectors may form functional oligomers at the action site.

### Example 3 CRC Proof of Concept in Prokaryotic Cells

In bacteria, inventors tested a system employing a negative selection approach with the antibiotic rifampicin. Rifampicin binds near the catalytic pocket of the subunit 13 of bacterial RNA polymerase, encoded by the rpoB gene, inhibiting transcription by physically blocking RNA elongation (22). Inventors defined mutations along a specific segment of the rpoB gene have been associated with rifampicin resistance. This region is known as rifampicin resistance determining region (23) (RRDR; FIG. 1C).

Four gRNAs were designed for these experiments targeting the template strand (TS1-TS4; FIG. 1C, Table S1), using catalytically dead Cas9 (dCas9) as DNA targeting module and one MS2 motif as recruiting module. The system expressing AID_MCP and dCas9 as effector and targeting modules, respectively, is noted as ^{A}CRCd. Treatment with ^{A}CRCd guided by gRNA TS4 resulted in survival fraction 35-fold higher than scramble treated cells. (FIGS. 1D and 1E). Sequence analysis of isolated colonies treated with ^{A}CRCd/rpoB_TS4 revealed that the system introduced a targeted C→T mutation in codon 531, changing a serine for phenylalanine, a mutation known to induce rifampicin resistance (23, 24) (FIG. 1F). The higher efficiency observed in TS4 treated cells might be due to the position of the targeted C within the protospacer (the unpaired DNA strand within the CRISPR R-loop), which in this case sits on position 8 from the 5′ end of the protospacer. On the other hand, TS2 and TS3 have target Cs at position 12 and 14 respectively, suggesting the distal positions from PAM motif within the protospacer region are favored.

Taken together, the data show that targeted nucleotide modification using an RNA-aptamer based effector recruitment mechanism is a potentially feasible approach for targeted base editing.

### Example 4 Engineering Individual Modules for System Optimization

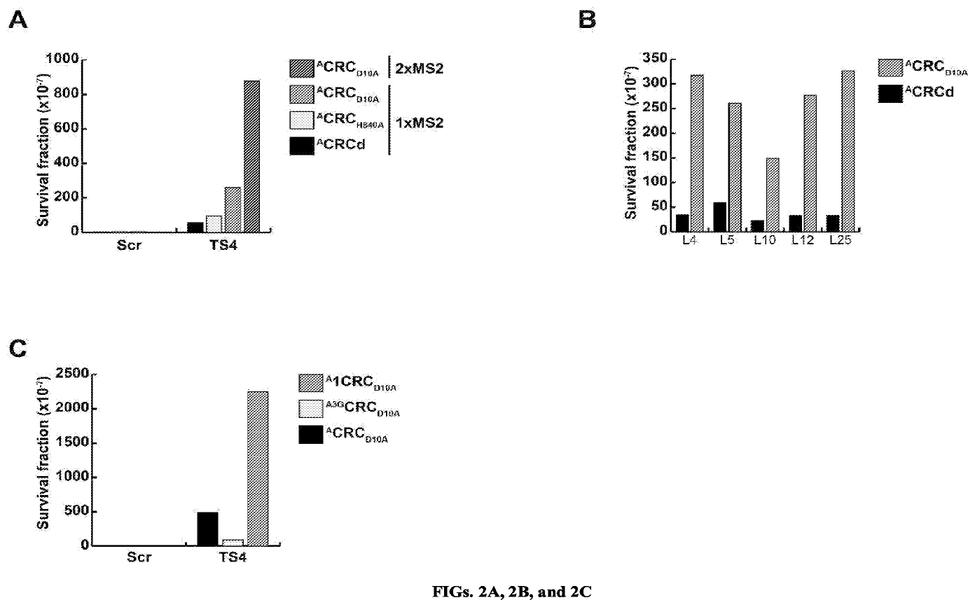

The positive results from the above exploratory experiments prompted to further engineer CRC system to increase its targeting efficiency using gRNA rpoB_TS4 for comparison. First, switching Cas9 module from dCas9 (^{A}CRCd) to nickase Cas9_{D10A}, which creates a single-strand DNA break (nick) at the complementary strand of the base editing target, resulted in 4.6-fold increase in the number of surviving colonies compared to ^{A}CRCd (FIG. 2A). Treatment with Cas9_{H840A} (^{A}CRC_{H840A}) modestly improved editing efficiency compared to ^{A}CRCd, with less than 2-fold increase in survival fraction (FIG. 2A). Remarkably, doubling the number of RNA aptamer sequence resulted in enhanced survival fraction, increasing the number of colonies over 360-fold compared to scramble treated cells, and 16-fold compared to ^{A}CRCd treated cells (FIG. 2A).

Although ^{A}CRC_{H840A} modestly increased the survival fraction compared to ^{A}CRCd (FIG. 2A), sequence analysis of individual clones revealed that it generated random mutations outside of the targeted region (within protospacer) at high frequency (FIG. 8A). While the latter systems targeted invariably the residue C1592 in codon 531, ^{A}CRC_{H840A} induced mutations not only on the target region, but also at several nucleotides upstream at high frequency (FIG. 8A). For this reason, it was decided to only adopt nCas_{D10A} in the recruitment module for further engineering and optimization.

To continue the optimization process, it was decided to engineer the system by testing different spatial configurations of the effector module, in ^{A}CRCd and ^{A}CRC_{D10A} systems. To this end, various linkers with different lengths and flexibilities were used to separate AID from MCP (Table S2).

TABLE S2
Effector module linker sequences used in bacterial experiments

| Linker name | Length (aa) | Sequence | SEQ ID NO |
|---|---|---|---|
| L4 | 4 | GSGS | 122 |
| L5 | 5 | GSGRA | 123 |
| L10 | 10 | GSGSGSGSGS | 124 |
| L12 | 12 | GGGGSGGGGSGGGGS | 125 |
| L25 | 25 | ELKTPLGDTTHTSPPCPAPELLGGP | 126 |

The flexible 25 amino acid linker (L25), derived from the hinge region of immunoglobin gamma 3 (IgG3), showed the highest efficiency, although the variations between the different linkers were relatively small, especially for ^{A}CRC_{D10A}, with 2-fold difference between the most and least efficient configuration (FIG. 2B). These results suggest that the spatial separation between AID and MCP in the effector module can be rather flexible.

Different types of cytidine deaminases can be incorporated into CRC system as effectors. Inventors tested two other proteins related to AID from the APOBEC family of cytidine deaminases: APOBEC1 and APOBEC3G (^{A1}CRC_{D10A} and ^{A3G}CRC_{D10A}, respectively). A¹CRC_{D10A} showed greater conversion efficiency, followed by ^{A}CRC_{D10A} and finally ^{A3G}CRC_{D10A} with the lowest activity (FIG. 2C). Sequencing analysis revealed that ^{A1}CRC_{D10A} induced a high rate of double mutants, whereas ^{A3G}CRC_{D10A} targeted nucleotides outside the protospacer at high frequency (FIG. 8B). Because of its wide activity window, ^{A3G}CRC_{D10A} was dropped from further optimization.

### Example 5 CRC System Corrects a Loss of Function Mutation in GFP Gene in Mammalian Cells

To determine if the CRC system works in mammalian cells, inventors tested the ^{A}CRC_{D10A} system in HEK293 cells. Mammalian expression of the various components was achieved by generating a multicistronic vector under the control of a CMV promoter, expressing AID_MCP fusion and nCas9_{D10A} separated by a self-cleavable 2A peptide (FIG. 9A). In cells, uracil DNA glycosylase (UNG) initiates the repair of U:G mismatches induced by cytidine deamination (25-27). To enhance nucleotide conversion efficiency at the target sites, a bacterial UNG inhibitor peptide (UGI) (28) was fused to nCas9, thus eliciting local UNG inhibition, a strategy to enhance efficiency of BE base editors. This mammalian CRC expression construct is noted as ^{A}CRCnu. The gRNA construct is driven by a U6 promoter and has two MS2 loops at the 3′ end of the CRISPR scaffold (2×MS2; FIG. 9B).

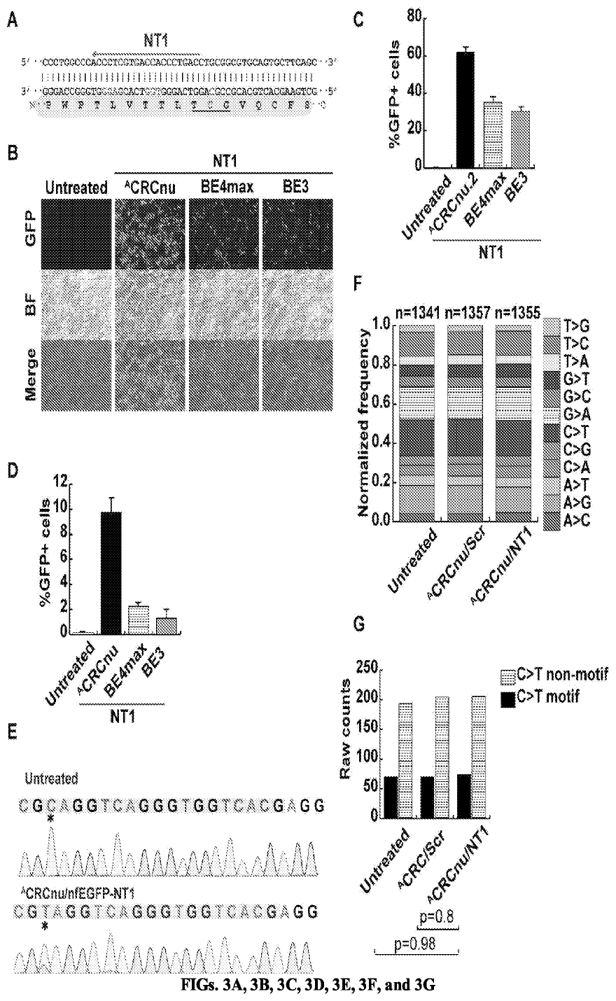

Inventors designed a GFP reporter that harbors an A→G point mutation along the chromophore sequence that results in tyrosine for cysteine mutation at position 66 (Y66C) (FIG. 3A). This mutation renders the protein non-fluorescent (nfEGFP), thus mimicking a loss of function (LOF) mutation. Inventors also designed a gRNA targeting the non-template strand (NT) around the mutation region (nfEGFP_NT1; FIG. 3A and Table S3).

First, inventors sought to correct the LOF mutation in extrachromosomal DNA. To this end, the target nfEGFP construct was transiently expressed in HEK 293T cells together with ^{A}CRCnu and nfEGFP_NT1 gRNA (FIG. 3B). For comparison, inventors tested third and fourth generation BE base editors, BE3 (13) and BE4max (29), side-by-side with ^{A}CRCnu. Higher GFP conversion was observed in ^{A}CRCnu than in BE4max and BE3 treated cells (FIG. 3B). Quantitation by flow cytometry revealed 62% GFP positive (GFP+) after ^{A}CRCnu/nfEGFP_NT1 treatment, whereas BE4max/nfEGFP_NT1 and BE3/nfEGFP_NT1 treatments resulted in 35% and 30% GFP+ cells, respectively (FIG. 3C).

To examine whether the system has base editing activity on chromosomal DNA sequence, low copy number mutant nfEGFP gene was stably integrated into HEK 293 genome (the resulting cell line was named nf2.16). The nf2.16 cells treated with ^{A}CRCnu, BE4max or BE3 targeted with nfEGFP_NT1 showed 9.8%, 2.3% and 1.3% correction efficiency, respectively (FIG. 3D). The GFP positive cells after treatment were sorted by fluorescent activated cell sorting analysis (FACS) followed by Sanger sequencing. The results confirmed the G→A conversion at the target base, restoring the wild type sequence (FIG. 3E).

Together, the results indicate that CRC system can edit extrachromosomal and chromosomal sequences. The data also demonstrate that CRC mediated base editing is feasible and efficient in mammalian cells in addition to prokaryotic cells.

### Example 6 Exome-Wide Analysis of Potential Off-Target Effects

To assess potential CRC mediated off-target activity at exome-wide level, nf2.16 cells underwent treatment with ^{A}CRCnu/nfEGFP_NT1, ^{A}CRCnu/scramble, or left untreated, were subjected to whole exome sequencing, which analyzes all exons across the genome with an average of 300× coverage. Analysis of point mutations showed no increase in global single nucleotide mutations in treated cells compared to untreated control (FIG. 3F). Because AID mutates cytosine residues preferentially within WRCH/DGYW motifs (where the underlined C and G are mutable positions) (30), to further confirm that expression of the effector (AID) does not increase point mutations, inventors examined the mutation rates of the AID motifs and non-motifs and compared between the treated and untreated cells. No difference was found between CRC treated and untreated samples, in both motif sequences and non-motif sequences (FIG. 3G). Taken together, the data show that the CRC system does not have significant effect in inducing global mutagenesis in the genome.

### Example 7 Base Editing by CRC at Endogenous Target Sequences

To determine CRC's ability to modify endogenous loci in the human genome, inventors targeted regions that have been extensively studied by conventional nuclease-dependent CRISPR (31, 32) as well as by BE base editing (13) (i.e., HEK 293 site 2, site 3 and site 4) and investigated the on-target efficacy, on-target indel formation rate, and potential off-target effect on homologous sequences. These sites and their targeting gRNAs are described in Table S4.

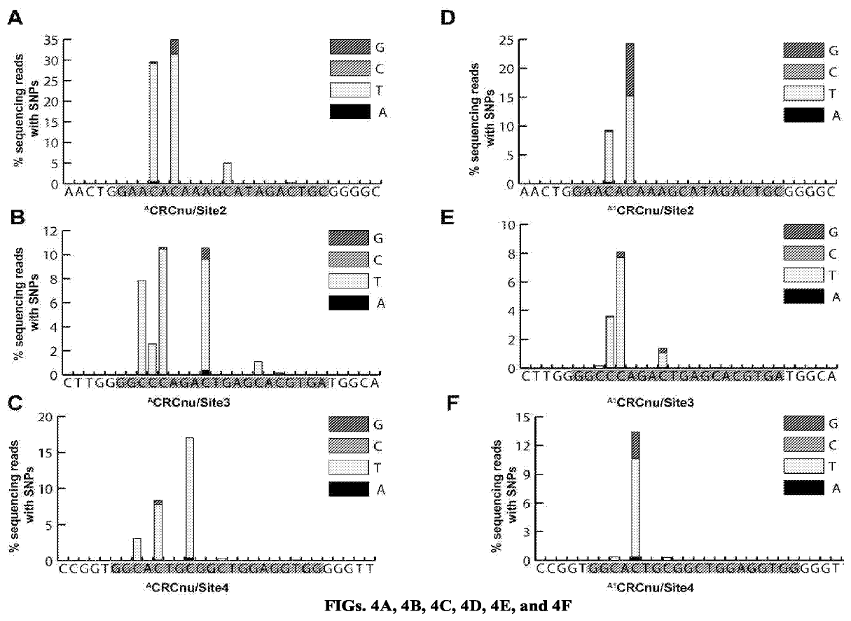

High throughput sequencing analysis revealed that CRC targeting at these sites resulted in significant C→T conversion, with high purity (i.e., low transversion frequency) (FIGS. 4A-C). ^{A}CRCnu treatment at site3 and site4 resulted in efficient nucleotide conversion (FIGS. 4B and 4C, respectively). These observations demonstrate that CRC is capable of targeting endogenous genomic sequences.

It is worth noting that, for these targets, ^{A}CRCnu construct (which expresses AID as effector) seems to have a wider activity window than the APOBEC1 based CRC editor ^{A1}CRCnu. In ^{A}CRCnu treatments, detectable editing is observed at Cs more distal to PAM (C11 in Site2, C9 in Site3 and C8 in Site4, FIGS. 4A-4C), whereas ^{A1}CRCnu (FIGS. 4D-4F) do not have significant activity at these positions. Because base editing is greatly constrained by PAM availability and the relative position of the target nucleotide within the protospacer, it could be advantageous to have systems with differences in activity window width.

### Example 8 Comparison of On-Target Indel Formation Rates and Off-Target Activities Between CRC and BE System

Cas9 nickases are largely considered safe since single strand breaks in DNA are well tolerated and efficiently repaired in cells (33-35). However, researchers have found that BE base editors that include nickases can still generate indels at the target site, albeit at much lower rates compared to conventional CRISPR approach (13, 29, 36). To determine the extent of indel formation after CRC treatment, inventors analyzed data to estimate the frequency of these events in treated and untreated cells. Indels were detected after CRC treatment with frequency comparable to indels induced by BE base editors (13, 36) but are both significantly lower than using a conventional CRISPR approach (36, 37), whereas untreated cells showed only background levels of indels (FIGS. 10A-10C). Note that the distribution and frequency of indels in treated cells correlates with gRNA target sites. In conclusion, CRC induces detectable indel at levels similar to BE base editors, both of which are at significantly lower levels as compared to the conventional CRISPR approach.

In order to estimate the extent of off-target activity of CRC and compare it to BE systems, inventors looked at selected known off-target sites of Site 2, Site 3, and Site 4, which were previously identified by chromatin immunoprecipitation of dCas9 bound to off-target sites (31), by GUIDE-seq method to determine wild type Cas9 off-target activity (32) and to evaluate BE base editors (13). The off-target sites probed are summarized on Table S5.

TABLE S5
HEK 293 site 2, site 3 and site 4 compared to their respective off-target sites
selected for off-target analysis. S2O1 is off-target site sequence for Site 2;
S3O1, S3O2, S3O3 are off-target site sequences for Site 3; S4O1, S4O2, S4O4 are
off-target site sequences for Site 4.

SEQ
ID

| NO: | | Target sequence | | | | | | | | | | | | | | | | | | | | PAM | | |
|---|---|---|---|---|---|---|---|---|---|---|---|---|---|---|---|---|---|---|---|---|---|---|---|---|
| 127 | Site2 | G | A | A | C | A | C | A | A | A | G | C | A | T | A | G | A | C | T | G | C | G | G | G |
| 128 | S2O1 | T | C | A | G | G | G | T | G | A | G | C | A | T | A | G | A | C | T | G | C | C | G | G |
| | | Target sequence | | | | | | | | | | | | | | | | | | | | PAM | | |
| 129 | Site3 | G | G | C | C | C | A | G | A | C | T | G | A | G | C | A | C | G | T | G | A | T | G | G |
| 130 | S3O1 | C | A | C | C | C | A | G | A | C | T | G | A | G | C | A | C | G | T | G | C | T | G | G |
| 131 | S3O2 | G | A | C | A | C | A | G | A | C | C | G | G | G | C | A | C | G | T | G | A | G | G | G |
| 132 | S3O3 | C | A | G | G | A | A | G | C | T | G | G | A | G | C | A | C | G | T | G | A | G | G | G |
| | | Target sequence | | | | | | | | | | | | | | | | | | | | PAM | | |
| 133 | Site4 | G | G | C | A | C | T | G | C | G | G | C | T | G | G | A | G | G | T | G | G | G | G | G |
| 134 | S4O1 | T | G | C | A | C | T | G | C | G | G | C | C | G | G | A | G | G | A | G | G | T | G | G |
| 135 | S4O2 | G | G | C | T | C | T | G | C | G | G | C | T | G | G | A | G | G | G | G | G | T | G | G |
| 136 | S4O4 | G | T | G | G | C | T | G | G | A | G | G | T | G | G | A | G | G | T | G | G | G | G | G |

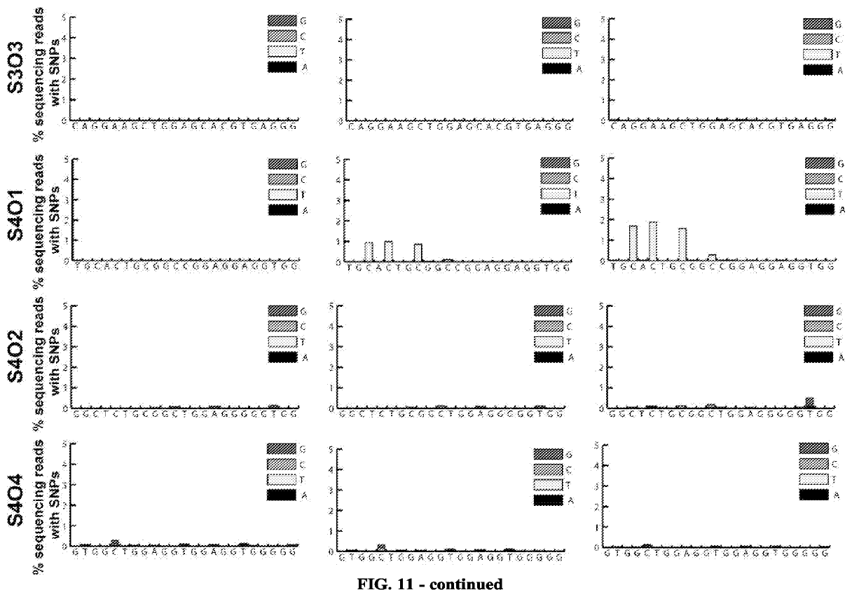

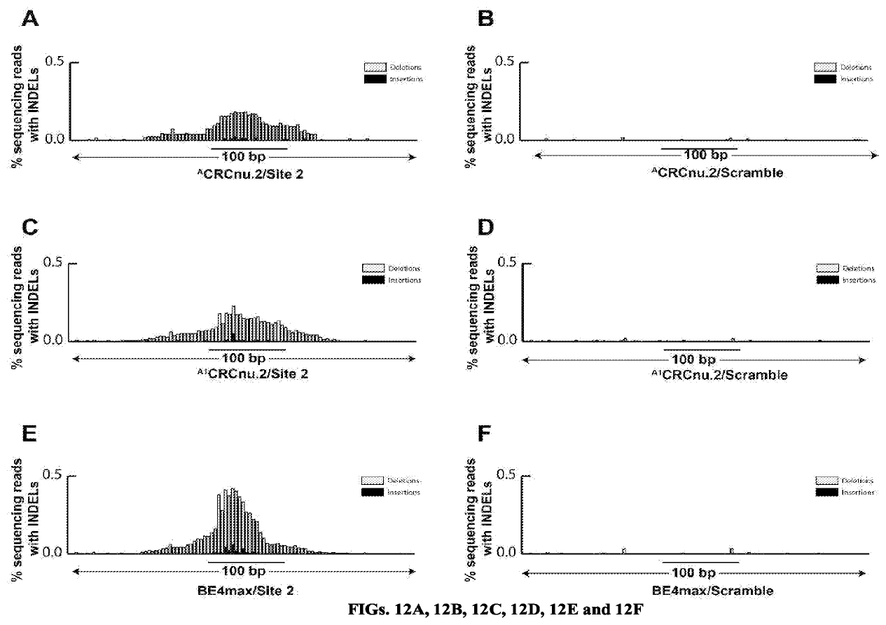

High throughput sequencing analysis revealed that the majority of off-target sites analyzed did not show editing activity (FIG. 11). In S4O1 (Site4 off-target site 1) inventors observed detectable C→T editing, however the frequency was much lower than the reported frequency at the same site for BE3 (i.e., less than 1% for C3, C5 and C8 in CRC treated cells, compared to 10% at C5 in BE3 treated cells (13)).

### Example 9 Construction of Second Generation CRC by Codon Optimization and Enhanced UNG Local Inhibition

Inventors generated second-generation CRC constructs by codon optimization, to enhance construct expression, and by appending an extra UGI copy to Cas9 to enhance local UNG inhibition and tested the impact on base editing efficiency as well as on-target indel formation and off-target effects. The resulting constructs are named ^{A}CRCnu.2 and ^{A1}CRCnu.2 (with AID and APOBEC1 as effectors, respectively; FIG. 9C).

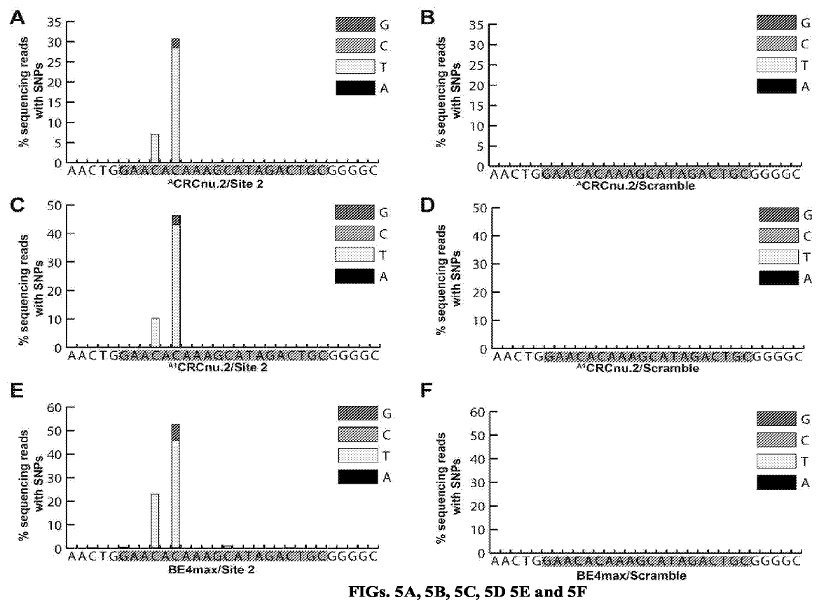

Inventors targeted HEK 293T site 2 with ^{A}CRCnu.2, ^{A1}CRCnu.2, and BE4max (29) for comparison. ^{A}CRCnu.2 and ^{A1}CRCnu.2 efficiencies reached 37% C→T at C4 and 41% at C6 for ^{A}CRCnu.2 (FIG. 5A), and 10% and 43% at the same Cs after ^{A1}CRCnu.2 treatment (FIG. 5B), which are dramatically increased as compared to their first-generation counterparts at the same site (FIGS. 4A and 4D), with maximal editing efficiency at around only 30% and 20% respectively. ^{A}CRCnu.2 induced 7% C→T at C11, confirming that AID has a broader activity window than APOBEC1 as a CRC effector at this site (FIG. 5A).

Off-target activity assessment of the optimized ^{A}CRCnu.2 system was also performed which revealed similar pattern as first generation CRC editor (FIG. 11) with undetectable base editing at most off-target sites (FIG. 11). It is interesting to note that while ^{A1}CRCnu.2 induced a comparable mutation rate at C6 as BE4 (43% vs. 44%), it induced much lower mutation rate at C4 (10% vs. 21%), indicating ^{A1}CRCnu.2 may have different preferable mutation sites within the protospace region from BE4max and can lead to a more discrete base editing pattern than BE4.

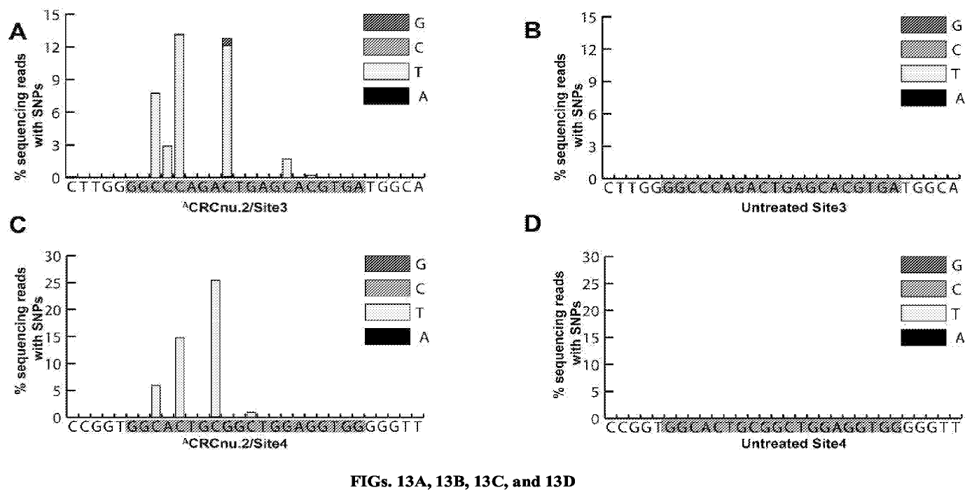

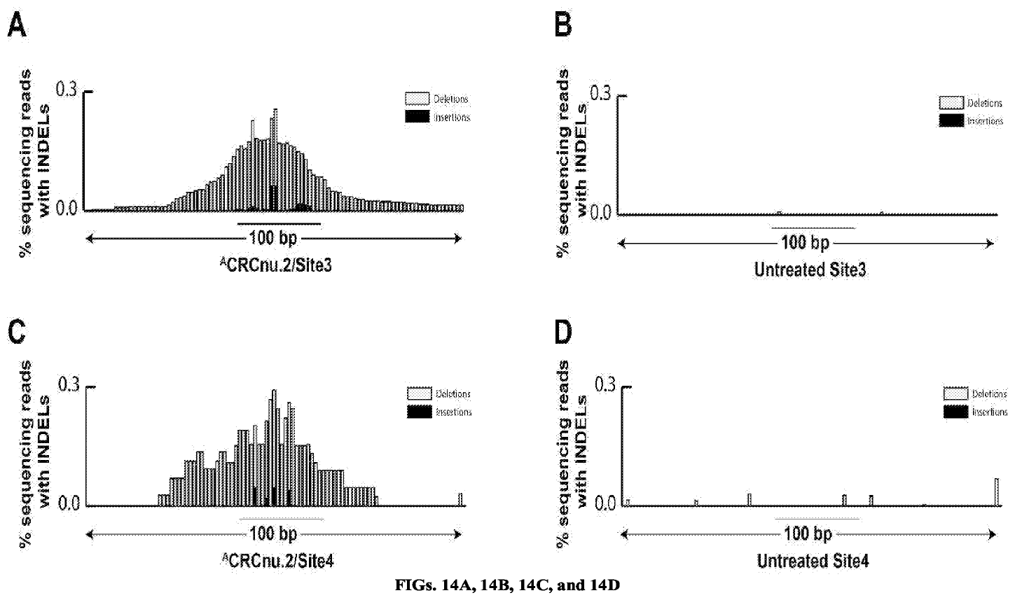

In addition, inventors targeted ^{A}CRCnu.2 to Site 3 and Site 4, which resulted in increased editing efficiencies compared to ^{A}CRCnu targeted to the same sites (FIG. 13 compared to FIGS. 4B-C), while maintaining low frequencies of indel formation (FIG. 14).

Together, the data show that the optimized, second-generation CRC base editors exhibited higher efficacy compared to the first generation CRC counterparts while maintaining low on-target indel formation rates and similar off-target profiles. Moreover, the data also support that the second generation CRC base editors operate at similar levels as BE base editor BE4max but they may have different activity windows and editing position preferences.

### Example 10 CRC Efficiently Mediates Targeted Gene Disruption by Induction of Premature Stop Codon

A major application for genome editing technologies in general is targeted gene disruption by DSB and activation of NHEJ, ultimately inducing frameshift mutations that introduce premature stop codons on the transcripts of targeted genes (38). Targeted gene inactivation could be an effective therapeutic strategy for removing a disease-causing gene product. CRC and other base editing strategy could provide a safer alternative of gene inactivation by directly editing CAG (Glutamine, Q), CAA (Glutamine, Q), CGA (Arginine, R) and TGG (Tryptophan, W) codons to TAG, TAA and TGA stop codons through a C to T mutation. Cytidine deaminase-mediated base editing by BE system has been harnessed to induce premature stop codons in a targeted manner, without requiring generation of DSB (39, 40).

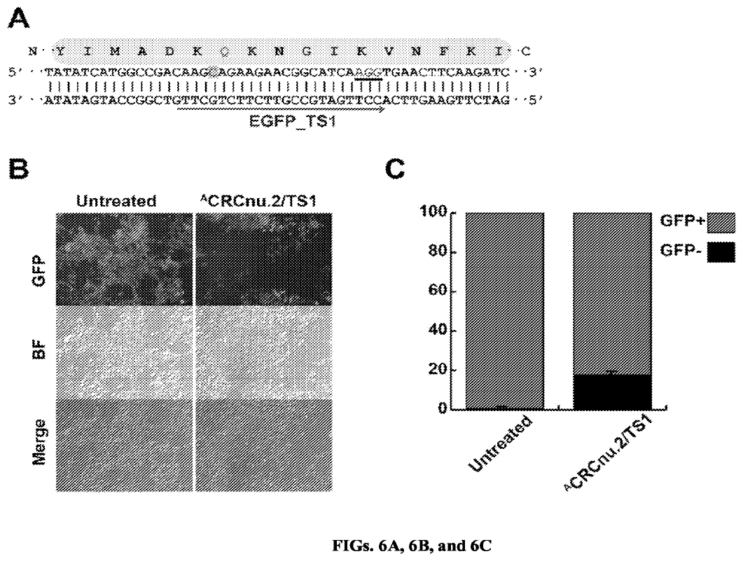

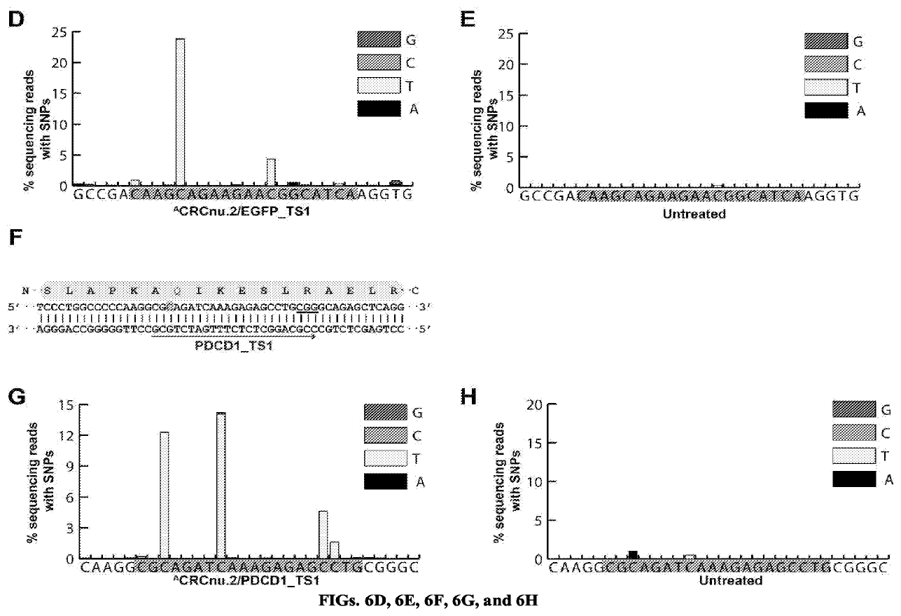

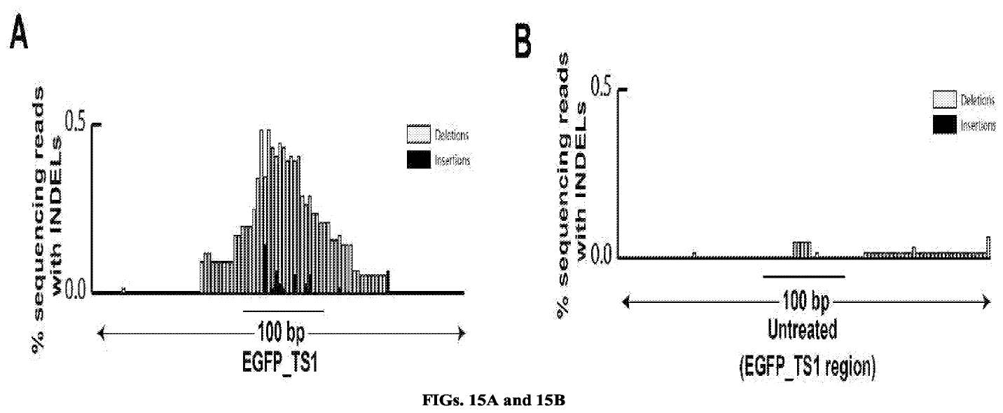

Inventors sought to test CRC's ability to induce stop codons on an EGFP reporter gene. One gRNA was designed targeting Q157 (EGFP_TS1) for generating stop codon at that position (FIG. 6A, Table S3). HEK293 cells stably expressing EGFP were targeted with TS1, resulting in efficient disruption of GFP expression (FIGS. 6B and 6C). Flow cytometry analysis revealed that TS1 induced 17.8% GFP negative cells (FIG. 6C). HTS analysis showed induction of stop codons at the target sites, confirming the observations by flow cytometry, with TS1 resulting in 24% C→T mutations at codon 157 (FIG. 6D). Low-level indel formation was detected in treated cells, following a similar pattern observed in previous experiments (FIG. 15).

Finally, to assess the ability of CRC to induce premature stop codons at an endogenous target, inventors sought to treat the PDCD1 loci with ^{A}CRCnu.2. PDCD1 gene encodes for the immune check point receptor PD1 (programmed cell death protein 1), which is a major target for immunotherapeutic strategies aimed to treat various types of cancer (41). Inventors designed one gRNA targeting codon 133, which encodes for glutamine (Q133) of the PD1 protein to induce a stop codon at this position (PDCD1_TS1; FIG. 6F, Table S4). ^{A}CRCnu.2 targeted with PDCD1_TS1 gRNA resulted in 14% C→T conversion at C3, converting codon Q133 (CAG) to stop codon (TAG) (FIG. 6G). Inventors observed bystander C editing with similar efficiency at C8 (FIG. 6G). This mutation occurs at the third position of codon 134, which does not change the isoleucine residue encoded by this codon. Together, these results provide proof-of-concept of efficient induction of targeted gene knockout by CRC base editing approach.

### Example 11 Different Species of APOBEC1 have Unexpected Widened Activity Window or Higher Activity at Certain Positions

In this example, different CRC systems were made using APOBEC1 of different species including those of rat, lizard (Anolis carolinensis), and bat (Myotis lucifugus). The effector protein and DNA sequences are shown below:

Anolis carolinensis APOBEC1 protein sequence
(SEQ ID NO: 137):
MEPEAFQRNFDPREFPECTLLLYEIHWDNNTSRNWCTNKPGLHAEENFL
QIFNEKIDIKQDTPCSITWFLSWSPCYPCSQAIIKFLEAHPNVSLEIKA
ARLYMHQIDCNKEGLRNLGRNRVSIMNLPDYRHCWTTFVVPRGANEDYW
PQDFLPAITNYSRELDSILQD
Anolis carolinensis APOBEC1 codon-optimized DNA
sequence
(SEQ ID NO: 138)
ATGGAGCCGGAGGCTTTTCAGCGCAACTTTGACCCTCGGGAATTTCCTG
AATGTACACTCCTCTTGTATGAGATCCACTGGGACAATAACACATCTAG
AAATTGGTGTACGAATAAGCCTGGGCTCCACGCTGAGGAGAATTTCTTG
CAGATATTTAATGAGAAAATTGACATTAAACAGGATACGCCGTGCTCTA
TAACATGGTTCCTTTCTTGGAGCCCCTGTTACCCTTGTAGCCAAGCAAT
AATAAAATTCTTGGAGGCACACCCGAATGTCAGTCTGGAGATTAAGGCT
GCGCGGCTGTATATGCATCAAATAGACTGTAACAAGGAGGGACTCAGAA
ATCTGGGCCGGAATCGAGTGTCAATAATGAACCTGCCTGATTATAGGCA
TTGCTGGACTACGTTTGTTGTGCCAAGGGGAGCAAACGAAGATTACTGG
CCACAAGACTTTCTGCCTGCGATCACAAATTACTCCCGAGAACTCGACT
CCATACTGCAGGAT
Myotis lucifugus APOBEC1 protein sequence
(SEQ ID NO: 139)
MASDAGSSAGDPTLRRRIEPWDFEAIFDPRELRKEACLLYEIKWGPCHK
IWRHSGKNTTRHVEVNFIEKITSERQFCSSTSCSIIWFLSWSPCWECSK
AITEFLRQRPGVTLVIYVARLYHHMDEQNRQGLRDLIKSGVTIQIMTTP
EYDYCWRNFVNYPPGKDTHCPMYPPLWMKLYALELHCIILSLPPCLMIS
RRCQKQLTWYRLNLQNCHYQQIPPHILLATAWI
Myotis lucifugus APOBEC1 codon-optimized DNA
sequence
(SEQ ID NO: 140)
ATGGCTTCAGACGCAGGCTCCTCCGCAGGGGATCCTACTTTGAGGCGAA
GGATCGAACCATGGGACTTCGAAGCAATTTTCGATCCTCGAGAGCTGAG
GAAAGAAGCCTGTCTGTTGTACGAAATTAAGTGGGGACCCTGTCACAAA
ATATGGCGGCATTCTGGCAAAAATACCACTAGACACGTCGAGGTTAACT
TTATCGAAAAAATCACAAGCGAGCGGCAATTCTGTTCTTCCACATCATG
TTCCATTATCTGGTTCCTTTCATGGAGCCCATGTTGGGAGTGCTCTAAA
GCAATAACCGAGTTTCTCAGGCAGAGACCTGGAGTAACTCTCGTAATCT
ACGTCGCCCGGCTCTACCACCACATGGATGAGCAAAATCGACAGGGGCT
TCGGGATCTCATTAAAAGTGGTGTCACGATACAAATTATGACGACTCCA
GAGTACGATTACTGCTGGCGGAACTTTGTGAACTACOCACCGGGCAAGG
ATACCCACTGTCCTATGTATCCACCCCTGTGGATGAAACTTTACGCACT
CGAGCTGCATTGTATCATTCTCTCCCTTCCACCGTGTCTCATGATCTCA
CGCAGGTGTCAAAAGCAGTTGACTTGGTACAGATTGAACCTTCAAAATT
GCCACTATCAACAGATTCCGCCTCATATTTTGCTGGCAACTGCGTGGAT
A.

These systems were examined in the same manner described above. The results are shown in FIGS. 16A-16D. As shown in the figures, these CRC systems utilizing a wide variety of cytidine deaminases from different species and different deaminase families, such as lizard Apobec1, show clear different activity windows and preference positions from any previously described base editing systems. These CRC systems can be used for nucleic acid modification (e.g., disease mutation corrections) unreached by other known effectors, in particular for targeting nucleotide close to PAM motif.

### Example 12 Different Species of AID or APOBEC1 have Unexpected Different Activity Windows or Higher Activity at Some Positions

In this example, CRC systems were made using AID or APOBEC1 of species including those of rat, lizard (Anolis carolinensis), and bat (Myotis lucifugus). The effector protein and DNA sequences are shown below:

### Anolis (Lizard) AID Ortholog

Shown below is an amino acid sequence of Anolis carolinensis single-stranded DNA cytosine deaminase (Activation induced cytidine deaminase, AID) fused to an MS2 coat protein (MCP):

(SEQ ID NO: 141)
PKKKRKVMMDSLLMKQKKFLYHFKNLRWAKGRHETYLCYVVKQRNSATS
CSLDFGYLRNKSGCHVEVLFLRYISTWDLDPRHCYRITWFTSWSPCYDC
ARHVADFLSAYPNLSLRIFAARLYFCEERNAEPEGLRRLHRAGAQTAIM
TFKDYFYCNNTFVENRKTTFKAWEGLHENSVRLARRLRRILLPLYEVDD
LRDAFRMLGLELKTPLGDTTHTSPPCPAPELLGGPMASNFTQFVLVDNG
GTGDVTVAPSNFANGIAEWISSNSRSQAYKVTCSVRQSSAQNRKYTIKV
EVPKGAWRSYLNMELTIPIFATNSDCELIVKAMQGLLKDGNPIPSAIAA
NSGIY

In the sequence above, the AID sequence (bold) is linked to the MCP sequence (underlined) via a hinge linker (italic), while the nuclear localization signal at the N-terminus is also underlined. Shown below is a codon optimized nucleotide sequence for expression the above protein in human cells:

(SEQ ID NO: 142)
CCCAAGAAGAAGCGGAAAGTGATGATGGACAGCCTTCTGATGAAGCAAA
AGAAATTTCTTTATCACTTCAAAAATCTGCGCTGGGCTAAGGGGAGGCA
CGAGACGTATCTCTGTTATGTAGTGAAACAAAGAAATAGTGCCACGTCT
TGTTCCCTTGATTTCGGTTATCTCCGAAACAAGAGCGGATGCCACGTTG
AAGTTCTGTTTTTGAGGTACATCAGCACGTGGGACCTCGACCCGAGACA
TTGCTACCGAATAACTTGGTTCACATCCTGGAGCCCCTGTTATGACTGC
GCTCGCCACGTAGCCGATTTTCTTAGTGCTTACCCTAACCTTTCACTCA
GGATTTTCGCCGCACGACTGTATTTCTGCGAGGAACGCAATGCTGAGCC
TGAAGGTCTCCGGAGGCTCCACCGAGCCGGGGCTCAAATAGCCATTATG
ACATTTAAGGATTACTTTTATTGTTGGAATACGTTTGTAGAGAACCGAA
AGACCACATTTAAGGCGTGGGAAGGTCTGCATGAGAATAGTGTCAGACT
TGCGAGGAGGCTGCGGAGGATCCTCTTGCCCCTCTATGAAGTAGATGAT
CTCCGCGATGCGTTCAGGATGTTGGGACTTGAGCTGAAGACACCCCTGG
GCGACACCACACACACCTCTCCACCTTGCCCAGCACCAGAGCTGCTGGG
AGGCCCTATGGCCAGCAACTTCACACAGTTTGTGCTGGTGGATAATGGA
GGAACCGGCGACGTGACAGTGGCACCATCTAACTTTGCCAATGGCATCG
CCGAGTGGATCAGCTCCAACTCTCGGAGCCAGGCCTATAAGGTGACCTG
TAGCGTGCGGCAGTCTAGCGCCCAGAATAGAAAGTATACAATCAAGGTG
GAGGTGCCTAAGGGCGCCTGGAGATCCTACCTGAACATGGAGCTGACCA
TCCCAATCTTTGCCACAAATTCTGATTGCGAGCTGATCGTGAAGGCCAT
GCAGGGCCTGCTGAAGGACGGCAACCCTATCCCAAGCGCCATCGCCGCC
AATAGCGGAATCTAC

### Anolis (Lizard) APOBEC1 Ortholog

Shown below is an amino acid sequence of Anolis carolinensis single-stranded DNA apolipoprotein B mRNA editing enzyme complex (APOBEC1) fused to an MCP:

(SEQ ID NO: 143)
PKKKRKVMEPEAFQRNFDPREFPECTLLLYEIHWDNNTSRNWCTNKPGL
HAEENFLQIFNEKIDIKQDTPCSITWFLSWSPCYPCSQAIIKFLEAHPN
VSLEIKAARLYMHQIDCNKEGLRNLGRNRVSIMNLPDYRHCWTTFVVPR
GANEDYWPQDFLPAITNYSRELDSILQDELKTPLGDTTHTSPPCPAPEL
LGGPMASNFTQFVLVDNGGTGDVTVAPSNFANGIAEWISSNSRSQAYKV
TCSVRQSSAQNRKYTIKVEVPKGAWRSYLNMELTIPIFATNSDCELIVK
AMQGLLKDGNPIPSAIAANSGIY

In the sequence above, the APOBEC1 sequence (bold) is linked to the MCP sequence (underlined) via a hinge linker (italic), while the nuclear localization signal at the N-terminus is also underlined. Shown below is a codon optimized nucleotide sequence for expression the above protein in human cells:

(SEQ ID NO: 144)
CCCAAGAAGAAGCGGAAAGTGATGGAGCCGGAGGCTTTTCAGCGCAACT
TTGACCCTCGGGAATTTCCTGAATGTACACTCCTCTTGTATGAGATCCA
CTGGGACAATAACACATCTAGAAATTGGTGTACGAATAAGCCTGGGCTC
CACGCTGAGGAGAATTTCTTGCAGATATTTAATGAGAAAATTGACATTA
AACAGGATACGCCGTGCTCTATAACATGGTTCCTTTCTTGGAGCCCCTG
TTACCCTTGTAGCCAAGCAATAATAAAATTCTTGGAGGCACACCCGAAT
GTCAGTCTGGAGATTAAGGCTGCGCGGCTGTATATGCATCAAATAGACT
GTAACAAGGAGGGACTCAGAAATCTGGGCCGGAATCGAGTGTCAATAAT
GAACCTGCCTGATTATAGGCATTGCTGGACTACGTTTGTTGTGCCAAGG
GGAGCAAACGAAGATTACTGGCCACAAGACTTTCTGCCTGCGATCACAA
ATTACTCCCGAGAACTCGACTCCATACTGCAGGATGAGCTGAAGACACC
CCTGGGCGACACCACACACACCTCTCCACCTTGCCCAGCACCAGAGCTG
CTGGGAGGCCCTATGGCCAGCAACTTCACACAGTTTGTGCTGGTGGATA
ATGGAGGAACCGGCGACGTGACAGTGGCACCATCTAACTTTGCCAATGG
CATCGCCGAGTGGATCAGCTCCAACTCTCGGAGCCAGGCCTATAAGGTG
ACCTGTAGCGTGCGGCAGTCTAGCGCCCAGAATAGAAAGTATACAATCA
AGGTGGAGGTGCCTAAGGGCGCCTGGAGATCCTACCTGAACATGGAGCT
GACCATCCCAATCTTTGCCACAAATTCTGATTGCGAGCTGATCGTGAAG
GCCATGCAGGGCCTGCTGAAGGACGGCAACCCTATCCCAAGCGCCATCG
CCGCCAATAGCGGAATCTAC

Myotis brandtii (Bat) AID Ortholog

Shown below is an amino acid sequence of Myotis brandtii single-stranded DNA cytosine deaminase (Activation induced cytidine deaminase, AID) fused to an MCP:

(SEQ ID NO: 145)
PKKKRKVMDSLLMKQRKFLYHFKNVRWAKGRHETYLCYVVKRRDSATSF
SLDFGHLRNKSGCHVELLFLRYISDWDLDPGRCYRVTWFTSWSPCYDCA
RHVADFLRGNPNLSLRIFTARLYFCEDYKAEPEGLRRLHRAGAQIAIMT
FKDYFYCWNTFVENRERTFRAWEGLHENSVRLSRQLRRILLPLYEVDDL
RDAFRTLGLELKTPLGDTTHTSPPCPAPELLGGPMASNFTQFVLVDNGG
TGDVTVAPSNFANGIAEWISSNSRSQAYKVTCSVRQSSAQNRKYTIKVE
VPKGAWRSYLNMELTIPIFATNSDCELIVKAMQGLLKDGNPIPSAIAAN
SGIY

In the sequence above, the AID sequence (bold) is linked to the MCP sequence (underlined) via a hinge linker (italic), while the nuclear localization signal at the N-terminus is also underlined. Shown below is a codon optimized nucleotide sequence for expression the above protein in human cells.

(SEQ ID NO: 146)
CCCAAGAAGAAGCGGAAAGTGATGGACTCTCTGCTGATGAAGCAGAGGA
AGTTTCTGTACCACTTCAAGAACGTGAGATGGGCCAAGGGCAGACACGA
AACCTATCTGTGCTACGTGGTGAAGAGGAGGGACAGCGCCACCTCCTTT
TCTCTGGATTTCGGCCACCTCAGAAACAAGTCCGGCTGCCACGTGGAGC
TGCTGTTTCTGAGGTACATCAGCGATTGGGATCTGGACCCCGGAAGATG
CTATAGAGTGACATGGTTCACCAGCTGGAGCCCTTGCTACGACTGCGCC
AGACACGTGGCCGACTTTCTGAGAGGCAACCCCAATCTGTCTCTGAGAA
TCTTCACCGCTAGACTGTACTTCTGCGAGGACTACAAGGCCGAGCCCGA
AGGACTGAGAAGGCTGCATAGAGCCGGCGCCCAGATCGCCATCATGACC
TTCAAGGACTACTTCTACTGCTGGAACACCTTCGTGGAAAATAGAGAGA
GAACCTTTAGAGCTTGGGAGGGCCTCCATGAGAACTCCGTGAGGCTGTC
TAGACAACTGAGGAGAATTCTGCTCCCTCTGTATGAGGTCGATGATCTG
AGAGACGCCTTCAGAACACTGGGACTGGAGCTGAAGACACCCCTGGGCG
ACACCACACACACCTCTCCACCTTGCCCAGCACCAGAGCTGCTGGGAGG
CCCTATGGCCAGCAACTTCACACAGTTTGTGCTGGTGGATAATGGAGGA
ACCGGCGACGTGACAGTGGCACCATCTAACTTTGCCAATGGCATCGCCG
AGTGGATCAGCTCCAACTCTCGGAGCCAGGCCTATAAGGTGACCTGTAG
CGTGCGGCAGTCTAGCGCCCAGAATAGAAAGTATACAATCAAGGTGGAG
GTGCCTAAGGGCGCCTGGAGATCCTACCTGAACATGGAGCTGACCATCC
CAATCTTTGCCACAAATTCTGATTGCGAGCTGATCGTGAAGGCCATGCA
GGGCCTGCTGAAGGACGGCAACCCTATCCCAAGCGCCATCGCCGCCAAT
AGCGGAATCTAC

gRNA Sequences

Shown below is a full gRNA construct coding sequence (target inserted at the underline/bold site with BbsI restriction digest)

(SEQ ID NO: 147)
CTAAATTGTAAGCGTTAATATTTTGTTAAAATTCGCGTTAAATTTTTGTTAAATCAGCTCATT
TTTTAACCAATAGGCCGAAATCGGCAAAATCCCTTATAAATCAAAAGAATAGACCGAGATAGG
GTTGAGTGTTGTTCCAGTTTGGAACAAGAGTCCACTATTAAAGAACGTGGACTCCAACGTCAA
AGGGCGAAAAACCGTCTATCAGGGCGATGGCCCACTACGTGAACCATCACCCTAATCAAGTTT
TTTGGGGTCGAGGTGCCGTAAAGCACTAAATCGGAACCCTAAAGGGAGCCCCCGATTTAGAGC
TTGACGGGGAAAGCCGGCGAACGTGGCGAGAAAGGAAGGGAAGAAAGCGAAAGGAGCGGGCGC
TAGGGCGCTGGCAAGTGTAGCGGTCACGCTGCGCGTAACCACCACACCCGCCGCGCTTAATGC
GCCGCTACAGGGCGCGTCCCATTCGCCATTCAGGCTGCGCAACTGTTGGGAAGGGCGATCGGT
GCGGGCCTCTTCGCTATTACGCCAGCTGGCGAAAGGGGGATGTGCTGCAAGGCGATTAAGTTG
GGTAACGCCAGGGTTTTCCCAGTCACGACGTTGTAAAACGACGGCCAGTGAGCGCGCGTAATA
CGACTCACTATAGGGCGAATTGGGTACCCGTCTCACAGGCGGATCGATCCAAGGTCGGGCAGG
AAGAGGGCCTATTTCCCATGATTCCTTCATATTTGCATATACGATACAAGGCTGTTAGAGAGA
TAATTGGAATTAATTTGACTGTAAACACAAAGATATTAGTACAAAATACGTGACGTAGAAAGT
AATAATTTCTTGGGTAGTTTGCAGTTTTAAAATTATGTTTTAAAATGGACTATCATATGCTTA
CCGTAACTTGAAAGTATTTCGATTTCTTGGCTTTATATATCTTGTGGAAAGGACGAAACACCG
GGTCTTCGAGAAGACCTGTTTTAGAGCTAGAAATAGCAAGTTAAAATAAGGCTAGTCCGTTAT
CAACTTGAAAAAGTGGCACCGAGTCGGTGCGGGAGCACATGAGGATCACCCATGTGCCACGAG
CGACATGAGGATCACCCATGTCGCTCGTGTTCCCTTTTTTTCTCCGCTGAGCGTACTGAGACG
CCGCGGTGGAGCTCCAGCTTTTGTTCCCTTTAGTGAGGGTTAATTGCGCGCTTGGCGTAATCA
TGGTCATAGCTGTTTCCTGTGTGAAATTGTTATCCGCTCACAATTCCACACAACATACGAGCC
GGAAGCATAAAGTGTAAAGCCTGGGGTGCCTAATGAGTGAGCTAACTCACATTAATTGCGTTG
CGCTCACTGCCCGCTTTCCAGTCGGGAAACCTGTCGTGCCAGCTGCATTAATGAATCGGCCAA
CGCGCGGGGAGAGGCGGTTTGCGTATTGGGCGCTCTTCCGCTTCCTCGCTCACTGACTCGCTG
CGCTCGGTCGTTCGGCTGCGGCGAGCGGTATCAGCTCACTCAAAGGCGGTAATACGGTTATCC
ACAGAATCAGGGGATAACGCAGGAAAGAACATGTGAGCAAAAGGCCAGCAAAAGGCCAGGAAC
CGTAAAAAGGCCGCGTTGOTGGCGTTTTTCCATAGGCTCCGCCCCCCTGACGAGCATCACAAA
AATCGACGCTCAAGTCAGAGGTGGCGAAACCCGACAGGACTATAAAGATACCAGGCGTTTCCC
CCTGGAAGCTCCCTCGTGCGCTCTCCTGTTCCGACCCTGCCGCTTACCGGATACCTGTCCGCC
TTTCTCCCTTCGGGAAGCGTGGCGCTTTCTCATAGCTCACGCTGTAGGTATCTCAGTTCGGTG
TAGGTCGTTCGCTCCAAGCTGGGCTGTGTGCACGAACCCCCCGTTCAGCCCGACCGCTGCGCC
TTATCCGGTAACTATCGTCTTGAGTCCAACCCGGTAAGACACGACTTATCGCCACTGGCAGCA
GCCACTGGTAACAGGATTAGCAGAGCGAGGTATGTAGGCGGTGCTACAGAGTTCTTGAAGTGG
TGGCCTAACTACGGCTACACTAGAAGGACAGTATTTGGTATCTGCGCTCTGCTGAAGCCAGTT
ACCTTCGGAAAAAGAGTTGGTAGCTCTTGATCCGGCAAACAAACCACCGCTGGTAGCGGTGGT
TTTTTTGTTTGCAAGCAGCAGATTACGCGCAGAAAAAAAGGATCTCAAGAAGATCCTTTGATC
TTTTCTACGGGGTCTGACGCTCAGTGGAACGAAAACTCACGTTAAGGGATTTTGGTCATGATC
AGAAGAACTCGTCAAGAAGGCGATAGAAGGCGATGCGCTGCGAATCGGGAGCGGCGATACCGT
AAAGCACGAGGAAGCGGTCAGCCCATTCGCCGCCAAGCTCTTCAGCAATATCACGGGTAGCCA
ACGCTATGTCCTGATAGCGGTCCGCCACACCCAGCCGGCCACAGTCGATGAATCCAGAAAAGC
GGCCATTTTCCACCATGATATTCGGCAAGCAGGCATCGCCATGGGTCACGACGAGATCCTCGC
CGTCGGGCATGCTCGCCTTGAGCCTGGCGAACAGTTCGGCTGGCGCGAGCCCCTGATGCTCTT
CGTCCAGATCATCCTGATCGACAAGACCGGCTTCCATCCGAGTACGTGCTCGCTCGATGCGAT
GTTTCGCTTGGTGGTCGAATGGGCAGGTAGCCGGATCAAGCGTATGCAGCCGCCGCATTGCAT
CAGCCATGATGGATACTTTCTCGGCAGGAGCAAGGTGAGATGACAGGAGATCCTGCCCCGGCA
CTTCGCCCAATAGCAGCCAGTCCCTTCCCGCTTCAGTGACAACGTCGAGCACAGCTGCGCAAG
GAACGCCCGTCGTGGCCAGCCACGATAGCCGCGCTGCCTCGTCTTGCAGTTCATTCAGGGCAC
CGGACAGGTCGGTCTTGACAAAAAGAACCGGGCGCCCCTGCGCTGACAGCCGGAACACGGCGG
CATCAGAGCAGCCGATTGTCTGTTGTGCCCAGTCATAGCCGAATAGCCTCTCCACCCAAGCGG
CCGGAGAACCTGCGTGCAATCCATCTTGTTCAATCATGCGAAACGATCCTCATCCTGTCTCTT
GATCGATCTTTGCAAAAGCCTAGGCCTCCAAAAAAGCCTCCTCACTACTTCTGGAATAGCTCA
GAGGCCGAGGCGGCCTCGGCCTCTGCATAAATAAAAAAAATTAGTCAGCCATGGGGCGGAGAA
TGGGCGGAACTGGGCGGAGTTAGGGGCGGGATGGGCGGAGTTAGGGGCGGGACTATGGTTGCT
GACTAATTGAGATGCATGCTTTGCATACTTCTGCCTGCTGGGGAGCCTGGGGACTTTCCACAC
CTGGTTGCTGACTAATTGAGATGCATGCTTTGCATACTTCTGCCTGCTGGGGAGCCTGGGGAC
TTTCCACACCCTAACTGACACACATTCCACAGCTGGTTCTTTCCGCCTCAGGACTCTTCCTTT
TTCAATATTATTGAAGCATTTATCAGGGTTATTGTCTCATGAGCGGATACATATTTGAATGTA
TTTAGAAAAATAAACAAATAGGGGTTCCGCGCACATTTCCCCGAAAAGTGCCAC

| Gene | Target | Sequence | SEQ ID NO |
|---|---|---|---|
| PD1 | Exon 2 | CGCAGATCAAAGAGAGCCTGCGG | 148 |
| HBF | Promoter 115-3 | CTTGACCAATAGCCTTGACAAGG | 149 |

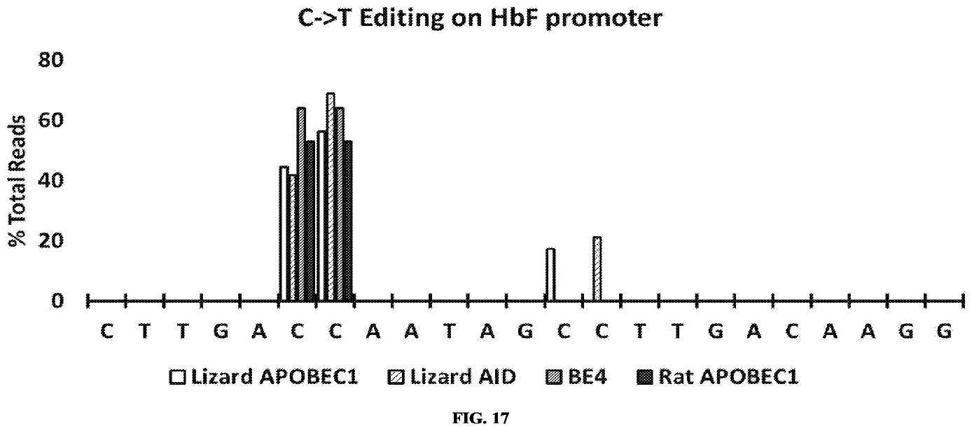

These lizard (Anolis carolinensis) and bat (Myotis lucifugus) AID or APOBEC1 were examined in the same manner described above. These effectors were constructed in second-generation CRC configuration (i.e. ^{LizardA}CRCnu.2, ^{LizardA1}CRCnu.2, ^{BatA}CRCnu.2 and ^{BatA1}CRCnu.2 constructs, where A refers to AID and A1 refers to APOBEC1). The results are shown in FIGS. 17-20.

First, it was found that lizard ^{LizardA1}CRCnu.2 system exhibited a wider activity window compared to rat ^{A1}CRCnu.2, making the cytidine nucleotide outside the activity window (positions 3 to 9 on the protospacer), in particular those cytidine proximal to PAM, accessible to the lizard APOBEC 1 effector.

FIG. 17 shows comparison of C to T conversion rates at a human fetal hemoglobin promoter locus in K562 cells by lizard ^{LizardA1}CRCnu.2, rat ^{A1}CRCnu.2, lizard ^{A}CRCnu.2, and BE4ₘₐₓ. systems. Briefly, K562 cells were transfected using Neon electroporation system with CRC expression vectors (expressing gRNA containing MS2 aptamer, MCP fused to lizard APOBEC1, lizard AID, rat APOBEC1, and nCas9D10A, or BE4max, total 1 μg DNA). Cells were grown for 72 hours after transfection; genomic DNA was isolated; the target fragment was amplified by PCR and subject to Sanger sequencing and high through put sequencing. The data show representative results from two independent experiments. The results showed that while all four effectors exhibited a high activity on cytidine at position C6 and C7 (consistent with the literature documented activity window between positions 3 to 9 on the protospacer) at this locus. In contrast, ^{LizardA1}CRCnu.2(lizard Apobec1) also had high activity at C3, and ^{LizardA}CRCnu.2 (lizard AID) had high activity at C14 (outside of the canonical activity window), in addition to high activities at C6 and C7.

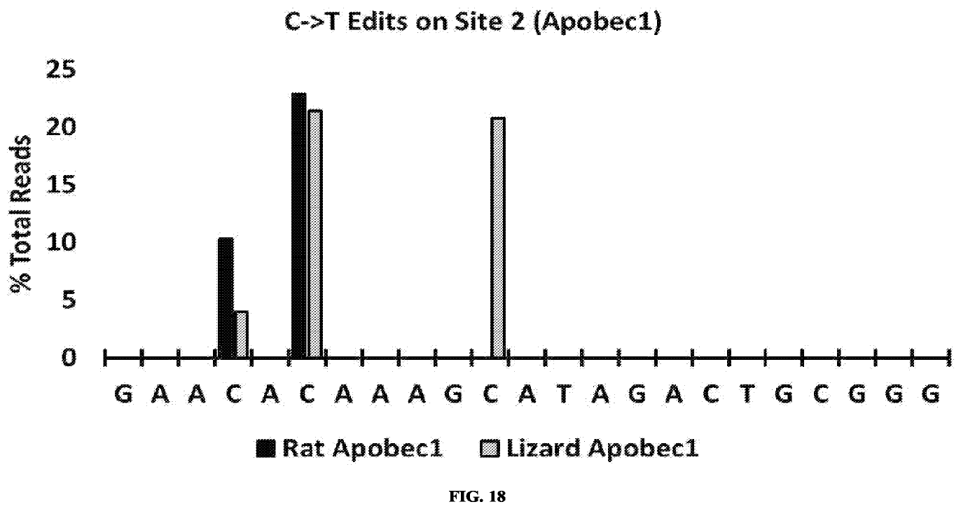

FIG. 18 shows a comparison of C to T conversion rates at the Site 2 locus in HEK293 cells by ^{LizardA1}CRCnu.2 and rat ^{A1}CRCnu.2 systems. HEK293 cells were transfected using Neon electroporation system with CRC expression vectors (expressing gRNA containing MS2 aptamer, MCP fused to lizard APOBEC1 or rat APOBEC1, and nCas9D10A, total 1 μg DNA). Cells were grown for 72 hours after transfection, genomic DNA was isolated, target fragment was amplified by PCR and subject to Sanger sequencing and high through put sequencing. ^{LizardA1}CRCnu.2 and rat ^{A1}CRCnu.2 were compared. The data show representative results from three independent experiments. The experiments showed that the rat ^{A1}CRCnu.2 construct exhibited high activity on cytidine at position C4 and C6 (consistent with the literature documented activity window between positions 3 to 9 on the protospacer) at this locus. In contrast, ^{LizardA1}CRCnu.2 also had high activity at C11 (outside the canonical activity window), in addition to high activities at C4 and C6.

As the PAM motif is at 3′ end of the sequences shown in charts the above results indicate that the cytidine proximal to the PAM motif could be targeted by ^{LizardA1}CRCnu.2 but not by rat ^{A1}CRCnu.2 or BE4max.

Second, it was found that ^{LizardA}CRCnu.2system, which expressed AID as effector, exhibited wider activity window compared to human ^{A}CRCnu.2, making the cytidine nucleotide outside the activity window (positions 3 to 9 on the protospacer), in particular those cytidine proximal to PAM, accessible to the lizard AID.

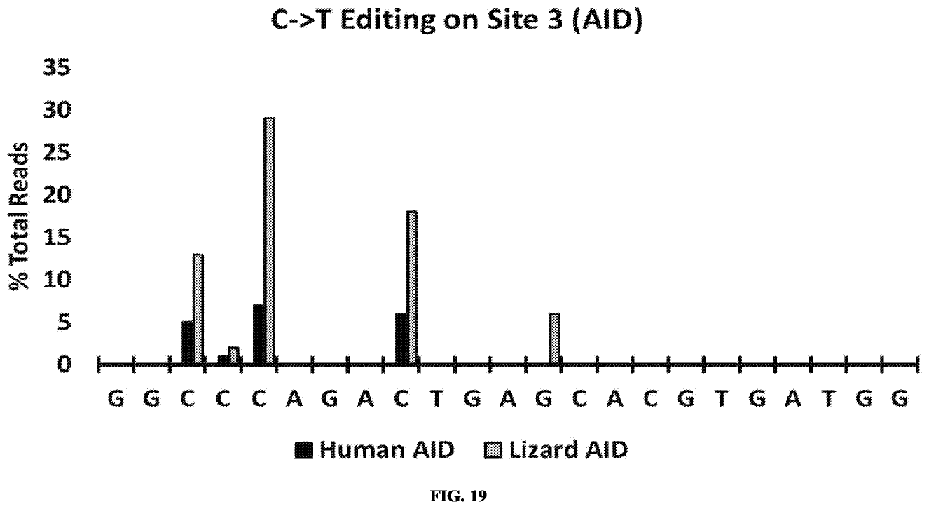

FIG. 19 shows comparison of C to T conversion rates at the Site 3 locus in HEK293 cells by ^{LizardA}CRCnu.2 (Lizard AID) and human ^{A}CRCnu.2 (human AID) systems. HEK293 cells were transfected using Neon electroporation system with CRC expression vectors (expressing gRNA containing MS2 aptamer, MCP fused to lizard AID or rat AID, and nCas9D10A, total 1 μg DNA). Cells were grown for 72 hours after transfection, genomic DNA was isolated, target fragment was amplified by PCR and subject to Sanger sequencing and high through put sequencing. Liard ^{LizardA}CRCnu.2 (gray) and human ^{A}CRCnu.2 (orange) were compared. The data show representative results from two independent experiments. The experiments showed that human ^{A}CRCnu.2 exhibited high activity on cytidine at position C3, C5, and C9 (consistent with the literature documented activity window between positions 3 to 9 on the protospacer) at this locus. In contrast, ^{LizardA}CRCnu.2 also had a high activity at C14 (outside the canonical window), in addition to high activities at C3, C5 and C9. As the PAM motif is at 3′ end of the sequence shown in charts, the results suggest that the cytidines proximal to the PAM motif could be targeted by ^{LizardA}CRCnu.2 but not by human ^{A}CRCnu.2.

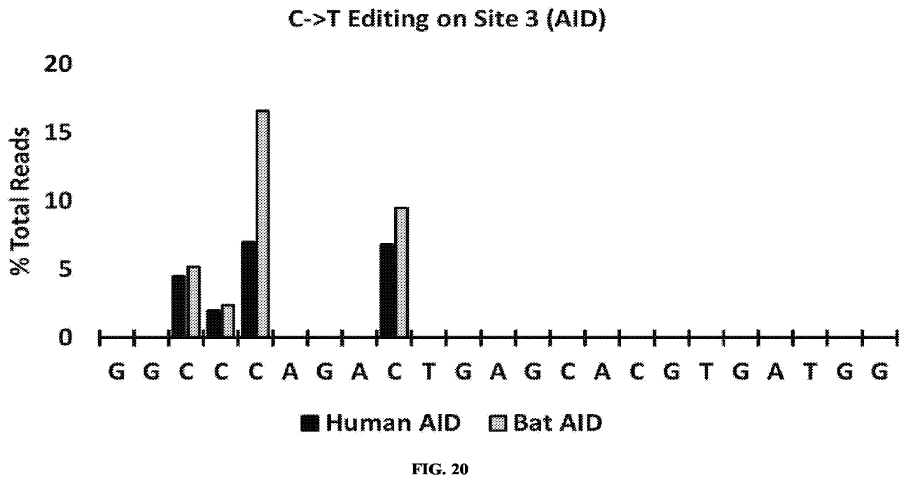

Third, it was found that ^{BatA}CRCnu.2 (bat AID) system exhibited higher base editing activity compared to human ^{A}CRCnu.2 (human AID) at certain loci. FIG. 20 shows comparison of C to T conversion rates at the Site 3 locus in HEK293 cells by ^{BatA}CRCnu.2 and human ^{A}CRCnu.2 systems. HEK293 cells were transfected using Neon electroporation system with CRC expression vectors (expressing gRNA containing MS2 aptamer, MCP fused to bat AID or rat AID, and nCas9D10A, total 1 μg DNA). Cells were grown for 72 hours after transfection; genomic DNA was isolated; the target fragment was amplified by PCR and subject to Sanger sequencing and high through put sequencing. ^{BatA}CRCnu.2 and human ^{A}CRCnu.2were compared. The data show representative results from two independent experiments. The results showed that ^{BatA}CRCnu.2 exhibited higher activity than human ^{A}CRCnu.2 on cytidine at position C3, C5, and C9, in particular at C5.

### Example 13 Comparison of Dead Cas and Nickase in Mammalian Cells

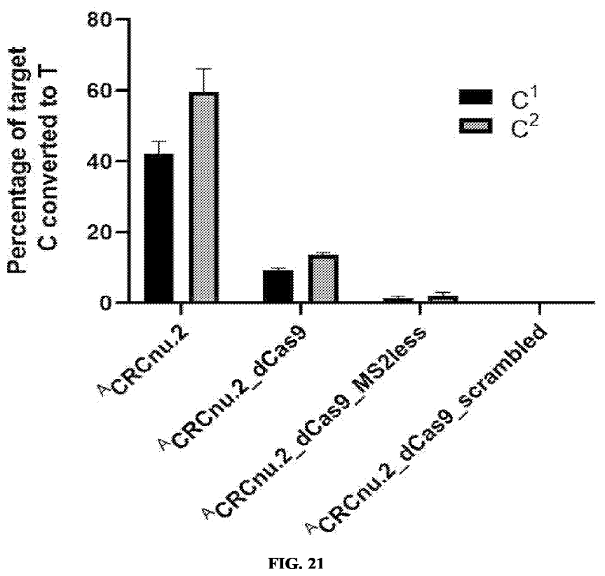

In this example, a study was carried out to compare dead Cas vs nickase in HEK cells, FIG. 21.

### Methods

Generation of dCas9

A catalytically dead Cas9 (dCas9) version of the ^{A}CRCnu.2 construct was generated by site-directed mutagenesis (SDM) using a Q5 site-directed mutagenesis kit (NEB: catalogue number—E0554S). A forward primer was designed to incorporate a 2 bp mismatch from the target nCas9 sequence which, following PCR amplification, changes codon 840 of nCas9 from CAT (histidine) to GCT (alanine). The _{H840A} mutation inactivates the HNH catalytic domain of Cas9, which, in combination with the D10A mutation already present in ^{A}CRCnu.2, generates a catalytically dead Cas9, which is no longer able to cleave dsDNA. The primers used for SDM are detailed in the table below. For the forward primer, the lower case “gc” represents the mismatch with the target sequence generating the CAT-GCT mutation at codon 840 of nCas9.

| Primer | Sequence (5′-3′) | SEQ ID NO |
|---|---|---|
| SDM forward primer | CGATGTGGACgcTATCGTGCCTCAGAGC | 150 |
| SDM reverse primer | TAGTCGGACAGCCGGTTG | 151 |

The PCR amplification was set-up as follows:

| Reagent | Volume (μl) |
|---|---|
| Q5 Hot start high-fidelity 2x master mix | 12.5 |
| Forward primer (10 μM) | 1.25 |
| Reverse primer (10 μM) | 1.25 |
| Water | 9 |
| Plasmid template (25 ng/μl) | 1 |
| Total | 25 |

The PCR reaction conditions were as follows:

| Step | Temperature | Time | |
|---|---|---|---|
| Initial denaturation | 98° C. | 30 | seconds |
| 30 cycles | 98° C. | 10 | seconds |
| | 65° C. | 30 | seconds |
| | 72° C. | 5 | minutes |
| Final extension | 72° C. | 2 | minutes |

Expression Plasmids

The components of the base editing system were expressed as a single polycistronic unit, whereby the Cas component and the MCP/deaminase fusion form two separate proteins by way of a T2A self-cleavage peptide.

The sgRNA component of the base editing system was expressed on a separate vector with expression of the sgRNA driven by the RNA polymerase III U6 promoter. The sgRNA was expressed as a single unit encompassing the crRNA and tracrRNA component of the Cas9 dual RNA system linked by an artificial tetra-loop. In addition, to enable recruitment of the deaminase, two copies of the RNA aptamer MS2 were tethered to the 3′ of the sgRNA through a fold-back dsRNA linker. As a control an sgRNA without the MS2 motifs (MS2less) was used, which due to the absence of the MCP recruiting aptamers should be incapable of editing the target locus. A poly-T termination signal was included at the 3′ of the sgRNA to catalyse the cessation of transcription. A list of the sgRNAs used and their sequences are shown in the table below:

| sgRNA name | sgRNA sequence (5′-3′) | SEQ ID NO |
|---|---|---|
| Site2_2xMS2 | gaac¹ac²aaagcatagactgcGTTTTAGAGCTAGAAAT | 152 |
| | AGCAAGTTAAAATAAGGCTAGTCCGTTATCA | |
| | ACTTGAAAAAGTGGCACCGAGTCGGTGCGGG | |
| | AGCACATGAGGATCACCCATGTGCCACGAGC | |
| | GACATGAGGATCACCCATGTCGCTCGTGTTC | |
| | CCTTTTTTT | |
| Site2_MS2less | gaac¹ac²aaagcatagactgcGTTTTAGAGCTAGAAAT | 153 |
| | AGCAAGTTAAAATAAGGCTAGTCCGTTATCA | |
| | ACTTGAAAAAGTGGCACCGAGTCGGTGCTTT | |
| | TTTT | |
| Scarmbled_2xMS2 | gcactaccagagctaactcaGTTTTAGAGCTAGAAATAG | 154 |
| | CAAGTTAAAATAAGGCTAGTCCGTTATCAAC | |
| | TTGAAAAAGTGGCACCGAGTCGGTGCGGGAG | |
| | CACATGAGGATCACCCATGTGCCACGAGCGA | |
| | CATGAGGATCACCCATGTCGCTCGTGTTCCCT | |
| | TTTTTT | |

In the table above, lower-case sequences denote the target specifying protospacer component of the sgRNA, whilst the upper-case sequences indicate the tracrRNA component of the sgRNA. Number superscripts denote C residues that reside within the target base editing window. A protospacer consisting of a scrambled sequence (Scrambled_2×MS2) was used as a negative control.

Cell Culture and Transfection

All transfection experiments were performed in HEK293 cells, and cells were cultured at 37° C. with 5% CO₂. The HEK293s were maintained in DMEM DMEM (Dulbecco's modified Eagle medium) supplemented with 10% FBS. To ensure a culture confluency of 70% for transfection, 24 hours prior to transfection HEK293s were seeded at a cell-density of 50,000 cells/well in a 24-well culture plate. 24 hours later the cells were lipid transfected with 200 ng of plasmid DNA (150 ng base editing/BE4max vector and 50 ng sgRNA expression vector) using LIPOFECTAMINE 3000 reagent (THERMOFISHER SCIENTIFIC: catalogue number —L3000015).

Cell Lysis and Flow Cytometry

72 hours post-transfection the media was aspirated, and the cells were washed once with PBS. The cells were then detached from the surface of the well with 100 μl of TrypLE express enzyme (THERMOFISHER SCIENTIFIC: catalogue number—12605010). The dissociated cells were then pelleted by centrifugation at 300×rpm for 5 minutes at room temperature, and subsequently resuspended in 100 μl of PBS. 20 μl of the cell suspension was transferred to a well of a 96 well plate containing 36 μl of DirectPCR lysis reagent (VIAGEN biotech: catalogue number—302-C), cell lysis was carried out under the following conditions: 55° C. for 30 minutes followed by 95° C. for 30 minutes. The remaining 80 μl of resuspended cells were transferred to a 96-well plate and collected by centrifugation at 300×rpm for 5 minutes at room temperature. The supernatant was discarded, and the cell pelleted were resuspended in 50 μl MACS buffer (MILTENYI BIOTEC) supplemented with 0.5% BSA in preparation for flow cytometry analysis. All flow cytometry was performed using the iQue3 (SARTORIUS).

PCR Amplification of Targeted Regions

1 μl of cell lysate was used per PCR reaction. The Q5 high-fidelity 2× master mix (NEB: catalogue number—M0491S) was used for amplification of sgRNA target sites, reaction mixes were set up as follows:

| Reagent | Volume | |
|---|---|---|
| Q5 2x master mix | 12.5 | μl |
| Forward primer (10 μM) | 1.25 | μl |
| Reverse primer (10 μM) | 1.25 | μl |
| Cell lysate | 1.0 | μl |
| Nuclease-free water | 9.0 | μl |
| Total | 25 | μl |

The PCR cycling parameters for amplification of the target site2 were as follows:

| Step | Temperature | Time | |
|---|---|---|---|
| Initial denaturation | 98° C. | 30 | seconds |
| 30 cycles | 98° C. | 10 | seconds |
| | 68° C. | 30 | seconds |
| | 72° C. | 30 | seconds |
| Final extension | 72° C. | 2 | minutes |

### Results

Cas9 nickase (nCas9-D10A) is the configuration of choice in base editing as nicking of the non-edited strand stimulates the cellular mismatch machinery, which uses the edited strand as a template for repair, and thereby shifts the balance of probability towards a C-to-T edit following replication. Introduction of the H840A mutation in nCas9 obliterates its nickase functionality, thereby preventing nicking of the non-edited DNA strand. The ability of the base editing system to achieve editing at a target locus with a catalytically dead Cas9 (dCas9) was measured. ^{A}CRCnu.2 was used as a template for generating a dCas9 version of the base editor, and editing efficiency was measured at site2.

As illustrated in FIG. 21, the data shows that the base editing system can achieve on target editing when using dCas9. The data shows that the highest level of editing at both C residues in the target sequence was achieved with a nCas9 (^{A}CRCnu.2), with C¹ showing 42% editing and C² showing 60% editing. Whilst using dCas9 (^{A}CRCdu.2) reduced editing activity (C¹=10%; C²=14%), it was still markedly higher than when an MS2less sgRNA was used (^{A}CRCdu.2_MS2less) or a non-targeting scrambled guide (^{A}CRCdu.2_scrambled). In conclusion, the use of a catalytically dead Cas9 is compatible with on target editing with the base editing system.

### REFERENCE

1. Fu Y F, et al. (2013) High-frequency off-target mutagenesis induced by CRISPR-Cas nucleases in human cells. Nature Biotechnology 31(9):822-+.
2. Singh P, Schimenti J C, & Bolcun-Filas E (2014) A Mouse Geneticist's Practical Guide to CRISPR Applications. Genetics.
3. Ran F A, et al. (2013) Double Nicking by RNA-Guided CRISPR Cas9 for Enhanced Genome Editing Specificity. Cell 154(6):1380-1389.
4. Tsai S Q et al. (2014) Dimeric CRISPR RNA-guided FokI nucleases for highly specific genome editing. Nat Biotech 32(6):569-576.
5. Guilinger J P, Thompson D B, & Liu D R (2014) Fusion of catalytically inactive Cas9 to FokI nuclease improves the specificity of genome modification. Nat Biotechnol 32(6):577-582.
6. Kleinstiver B P, et al. (2016) High-fidelity CRISPR-Cas9 nucleases with no detectable genome-wide off-target effects. Nature 529(7587):490-495.
7. Slaymaker I M, et al. (2016) Rationally engineered Cas9 nucleases with improved specificity. Science 351(6268):84-88.
8. Kosicki M, Tomberg K, & Bradley A (2018) Repair of double-strand breaks induced by CRISPR-Cas9 leads to large deletions and complex rearrangements. Nature Biotechnology 36:765.
9. Rivera-Torres N, Banas K, Bialk P, Bloh K M, & Kmiec E B (2017) Insertional Mutagenesis by CRISPR/Cas9 Ribonucleoprotein Gene Editing in Cells Targeted for Point Mutation Repair Directed by Short Single-Stranded DNA Oligonucleotides. PloS one 12(1):e0169350.
10. Corrigan-Curay J, et al. (2015) Genome editing technologies: defining a path to clinic. Mol Ther 23(5):796-806.
11. Cox D B, Platt R J, & Zhang F (2015) Therapeutic genome editing: prospects and challenges. Nature medicine 21(2):121-131.
12. Iyama T & Wilson D M (2013) DNA repair mechanisms in dividing and non-dividing cells. DNA repair 12(8):620-636.
13. Komor A C, Kim Y B, Packer M S, Zuris J A, & Liu D R (2016) Programmable editing of a target base in genomic DNA without double-stranded DNA cleavage. Nature 533(7603):420-+.
14. Cox D B, et al. (2017) RNA editing with CRISPR-Cas13. Science 358(6366):1019-1027.
15. Zalatan J G, et al. (2015) Engineering complex synthetic transcriptional programs with CRISPR RNA scaffolds. Cell 160(1-2):339-350.
16. Konermann S, et al. (2015) Genome-scale transcriptional activation by an engineered CRISPR-Cas9 complex. Nature 517(7536):583-588.
17. Wang S, Su J-H, Zhang F, & Zhuang X (2016) An RNA-aptamer-based two-color CRISPR labeling system. Scientific reports 6:26857.
18. Qin P, et al. (2017) Live cell imaging of low- and non-repetitive chromosome loci using CRISPR-Cas9. Nature communications 8:14725.
19. Jin S, Collantes, J C (2017) Nuclease-Independent Targeted Gene Editing Platform and Uses Thereof. PCT/US2016/042413 (Priority date: 15 Jul. 2015)
20. Hess G T, et al. (2016) Directed evolution using dCas9-targeted somatic hypermutation in mammalian cells. Nature methods 13(12):1036.
21. Liu L D, et al. (2018) Intrinsic nucleotide preference of Diversifying Base editors guides antibody ex vivo affinity maturation. Cell Reports 25(4):884-892. e883.
22. Campbell E A, et al. (2001) Structural Mechanism for Rifampicin Inhibition of Bacterial RNA Polymerase. Cell 104(6):901-912.
23. Goldstein B P (2014) Resistance to rifampicin: a review. J Antibiot (Tokyo) 67(9):625-630.
24. Xu M, Zhou Y N, Goldstein B P, &Jin D J (2005) Cross-Resistance of Escherichia coli RNA Polymerases Conferring Rifampin Resistance to Different Antibiotics. Journal of Bacteriology 187(8):2783-2792.
25. Petersen-Mahrt S K, Harris R S, & Neuberger M S (2002) AID mutates E. coli suggesting a DNA deamination mechanism for antibody diversification. Nature 418(6893):99-104.
26. Krokan H E & Bjørås M (2013) Base excision repair. Cold Spring Harbor perspectives in biology 5(4):a012583.
27. Jacobs A L & Schär P (2012) DNA glycosylases: in DNA repair and beyond. Chromosoma 121(1):1-20.
28. Mol C D, et al. (1995) Crystal structure of human uracil-DNA glycosylase in complex with a protein inhibitor: protein mimicry of DNA. Cell 82(5):701-708.
29. Koblan L W, et al. (2018) Improving cytidine and adenine base editors by expression optimization and ancestral reconstruction. Nature Biotechnology.
30. Odegard V H & Schatz D G (2006) Targeting of somatic hypermutation. Nat Rev Immunol 6(8):573-583.
31. Kuscu C, Arslan S, Singh R, Thorpe J, & Adli M (2014) Genome-wide analysis reveals characteristics of off-target sites bound by the Cas9 endonuclease. Nature Biotechnology 32:677.
32. Tsai S Q et al. (2014) GUIDE-seq enables genome-wide profiling of off-target cleavage by CRISPR-Cas nucleases. Nature Biotechnology 33:187.
33. Caldecott K W (2001) Mammalian DNA single-strand break repair: an X-ra(y)ted affair. Bioessays 23(5):447-455.
34. Caldecott K W (2008) Single-strand break repair and genetic disease. Nature Reviews Genetics 9(8):619-631.
35. Caldecott K W (2014) DNA single-strand break repair. Experimental cell research 329(1):2-8.
36. Rees H A & Liu D R (2018) Base editing: precision chemistry on the genome and transcriptome of living cells. Nature Reviews Genetics:1.
37. Chakrabarti A M, et al. (2019) Target-Specific Precision of CRISPR-Mediated Genome Editing. Molecular cell 73(4):699-713 e696.
38. Sander J D & Joung J K (2014) CRISPR-Cas systems for editing, regulating and targeting genomes. Nat Biotechnol 32(4):347-355.
39. Kuscu C, et al. (2017) CRISPR-STOP: gene silencing through base-editing-induced nonsense mutations. Nature methods 14:710.
40. Billon P, et al. (2017) CRISPR-Mediated Base Editing Enables Efficient Disruption of Eukaryotic Genes through Induction of STOP Codons. Molecular cell 67(6):1068-1079.e1064.
41. Pardoll D M (2012) The blockade of immune checkpoints in cancer immunotherapy. Nature Reviews Cancer 12:252.
42. Grünewald J, et al. (2019) Transcriptome-wide off-target RNA editing induced by CRISPR-guided DNA base editors. Nature 569(7756):433.
43. Duan D, Yue Y, & Engelhardt J F (2001) Expanding AAV packaging capacity with trans-splicing or overlapping vectors: a quantitative comparison. Molecular therapy 4(4):383-391.
44. Carvalho L S, et al. (2017) Evaluating efficiencies of dual AAV approaches for retinal targeting. Frontiers in neuroscience 11:503.
45. Grieger J C & Samulski R J (2005) Packaging Capacity of Adeno-Associated Virus Serotypes: Impact of Larger Genomes on Infectivity and Postentry Steps. Journal of Virology 79(15):9933-9944.
46. Shapiro M B & Senapathy P (1987) RNA splice junctions of different classes of eukaryotes: sequence statistics and functional implications in gene expression. Nucleic acids research 15(17):7155-7174.
47. Baralle D & Baralle M (2005) Splicing in action: assessing disease causing sequence changes. Journal of medical genetics 42(10):737-748.
48. Gapinske M, et al. (2018) CRISPR-SKIP: programmable gene splicing with single base editors. Genome biology 19(1):107.
49. Kabadi A M, Ousterout D G, Hilton I B, & Gersbach C A (2014) Multiplex CRISPR/Cas9-based genome engineering from a single lentiviral vector. Nucleic acids research 42(19):e147-e147.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

1. A system comprising:
(i) a sequence-targeting component or a polynucleotide encoding the same, said component comprising a target fusion protein having
(a) a sequence-targeting protein, and
(b) a first uracil DNA glycosylase (UNG) inhibitor peptide (UGI);
(ii) an RNA scaffold, or a DNA polynucleotide encoding the same, said scaffold comprising
(a) a nucleic acid-targeting motif comprising a guide RNA sequence that is complementary to a target nucleic acid sequence,
(b) an RNA motif capable of binding to the sequence-targeting protein, and
(c) a first recruiting RNA motif, and
(iii) a first effector fusion protein, or a polynucleotide encoding the same, said protein comprising
(a) a first RNA binding domain capable of binding to the first recruiting RNA motif,
(b) a linker, and
(c) an effector domain,
wherein the first effector fusion protein or the effector domain has a cytosine deamination activity or adenosine deamination activity.

2. The system of claim 1, wherein the target fusion protein further comprises two or more UGIs.

3.-4. (canceled)

5. The system of claim 1, wherein the sequence-targeting component or the first effector fusion protein comprises one or more nuclear localization signals (NLSs).

6. (canceled)

7. The system of claim 1, wherein the sequence-targeting protein is a CRISPR protein.

8. The system of claim 1, wherein the sequence-targeting protein does not have a nuclease activity.

9. The system of claim 1, wherein the sequence-targeting protein comprises the sequence of dCas9 or nCas9 of a species selected from the group consisting of Streptococcus pyogenes, Streptococcus agalactiae, Staphylococcus aureus, Streptococcus thermophilus, Streptococcus thermophilus, Neisseria meningitidis, and Treponema denticola.

10. The system of claim 1, wherein the first recruiting RNA motif and the first RNA binding domain are a pair selected from the group consisting of:
a telomerase Ku binding motif and Ku protein or an RNA-binding section thereof,
a telomerase Sm7 binding motif and Sm7 protein or an RNA-binding section thereof,
a MS2 phage operator stem-loop and MS2 coat protein (MCP) or an RNA-binding section thereof,
a PP7 phage operator stem-loop and PP7 coat protein (PCP) or an RNA-binding section thereof,
a SfMu phage Com stem-loop and Com RNA binding protein or an RNA-binding section thereof, and
a chemically modified version of the above aptamers and their corresponding aptamer ligand or an RNA-binding section thereof and a non-natural RNA aptamer and corresponding aptamer ligand or an RNA-binding section thereof.

11.-12. (canceled)

13. An isolated nucleic acid encoding one or more of components (i)-(iii) of the system of claim 1.

14. An expression vector or a host cell comprising the nucleic acid of claim 13.

15. A method of site-specific modification of a target DNA, comprising contacting the target nucleic acid with the system of claim 1.

16. The method of claim 15, wherein the target nucleic acid is in a cell.

17.-18. (canceled)

19. The method of claim 16, wherein the cell is selected from the group consisting of an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, a mammalian cell, a pig cell, a cow cell, a goat cell, a sheep cell, a rodent cell, a rat cell, a mouse cell, a horse cell, a non-human primate cell, and a human cell.

20. (canceled)

21. The method of claim 19, wherein the cell is in or derived from a human or non-human subject.

22. The method of claim 21, wherein the human or non-human subject has a genetic mutation of a gene.

23. The method of claim 22, wherein the subject has a disorder caused by the genetic mutation or is at risk of having the disorder.

24. The method of claim 21, wherein said site-specific modification corrects a genetic mutation or inactivates the expression of a gene or changes the expression levels of a gene or changes intron-exon splicing.

25. The method of claim 21, wherein the subject has a pathogen or is at risk of exposing to the pathogen.

26. The method of claim 25, wherein said site-specific modification inactivates a gene of the pathogen.

27. A kit comprising the system of claim 1.

28. (canceled)

29. A genetically engineered isolated cell obtained according to the method of claim 15.

30.-32. (canceled)

33. A pharmaceutical composition comprising an effective amount of the cell of claim 29 and a pharmaceutically acceptable carrier.
